(19) United States
(12) Patent Application Publication
(10) Pub. No.: US 2025/0171769 A1
(43) Pub. Date: May 29, 2025

(54) SPATIAL TRANSPOSITION-BASED RNA SEQUENCING LIBRARY PREPARATION METHOD

(71) Applicant: ILLUMINA, INC., San Diego, CA (US)

(72) Inventors: Lena Storms, San Diego, CA (US); Craig April, San Diego, CA (US); Mats Ekstrand, San Diego, CA (US); Kerou Zhang, San Diego, CA (US); Yao Xiao, San Diego, CA (US); Brittany Flowers, San Diego, CA (US); Anustup Poddar, San Diego, CA (US); Andrea Manzo, San Diego, CA (US); Olivia Ghazinejad, San Diego, CA (US); Fei Shen, San Diego, CA (US); Brian Mather, San Diego, CA (US); Se Min Canon, Philadelphia, PA (US); Andrew Ostrow, San Diego, CA (US)

(21) Appl. No.: 18/851,404

(22) PCT Filed: Dec. 22, 2023
(86) PCT No.: PCT/US23/85743

§ 371 Date: Sep. 26, 2024

Related U.S. Application Data
(60) Provisional application No. 63/604,667, filed on Nov. 30, 2023.
(60) Provisional application No. 63/586,872, filed on Sep. 29, 2023.
(60) Provisional application No. 63/477,103, filed on Dec. 23, 2022.

(51) Int. Cl. C12N 15/10; C12Q 1/44; C12Q 1/6806; C12Q 1/6834; C12Q 1/6855; C12Q 1/686

(52) U.S. Cl. CPC C12N 15/1065; C12N 15/1096; C12Q 1/44; C12Q 1/6806; C12Q 1/6834; C12Q 1/6855; C12Q 1/686; G01N 2333/922

(57) ABSTRACT

An RNA sequencing library preparation process that that utilized transposition with or with a template switch oligonucleotide to generate the libraries having UMIs and spatial barcode information, and methods for improving RNA library preparation from tissue samples using template switching and thermal amplification to improve RNA library quality.

### CROSS REFERENCE TO RELATED APPLICATIONS

The present application claims the priority benefit of U.S. Provisional Patent Application No. 63/477,103, filed Dec. 23, 2022, U.S. Provisional Patent Application No. 63/586,872, filed Sep. 29, 2023, and U.S. Provisional Patent Application No. 63/604,667, filed Nov. 30, 2023, each of which is incorporated by reference in its entirety.

### INCORPORATION BY REFERENCE OF SEQUENCE DISCLOSURE

The Sequence Listing, which is a part of the present disclosure, is submitted concurrently with the specification as a computer readable file. The name of the file containing the Sequence Listing is “IP-2528-PC_SeqListing.xml”, which was created on Dec. 15, 2023, and is 19,146 bytes in size. The subject matter of the Sequence Listing is incorporated herein in its entirety by reference.

### FIELD

The disclosure relates to spatial transposition based methods for preparing an RNA sequence library, and in particular, to methods for preparing RNA sequencing libraries with spatial transpositions based method both with and without a template switch oligonucleotide.

### BACKGROUND

Spatial transcriptomic enables highly multiplexed, spatially located gene expression analysis from fresh frozen and formalin-fixed paraffin-embedded (FFPE) tissue samples. In order to generate spatial sequencing libraries, an on-surface library preparation method must be used to spatially capture and barcode transcripts from a tissue sample. Sequencing libraries must also include unique molecular identifies (UMIs) and sample indices, while maintaining an optimal length for sequencing. Current spatial workflows require fragmentation to generate libraries of optimal fragment size for sequencing and contain UMI information on a barcoded surface. Current on-market spatial workflows capture and convert <1% mRNA within a tissue section.

### SUMMARY

Presented here are methods to generate higher capture and spatial library conversion from preserved tissue samples, e.g., frozen or FFPE tissue samples. In situ polyadenylation can enable capture of fragmented FFPE RNA on oligo-dT surface. Also provided herein are improved methods to synthesize cDNA from isolated RNA transcripts to improve the overall synthesis and alignment quality of the RNA sequences and preparation of a spatial transcriptomics library.

Provided herein is a method for preparing an RNA sequence library in accordance with the disclosure can include mounting a tissue sample on a substrate comprising a plurality of capture oligonucleotides, wherein the capture oligonucleotides comprise one or more a gene-specific capture sequences and library barcode information comprising a spatial barcode sequence (SBC); capturing mRNA transcripts on the substrate with the capture oligonucleotides; contacting the substrate with a first strand synthesis mix comprising a reverse transcriptase and a template switch oligonucleotide (TSO) under conditions to generate a first strand comprising a first cDNA complementary to the mRNA transcripts, and a TSO complement hybridized to the 5′ end of the first cDNA, wherein the reverse transcriptase incorporates untemplated cytosine nucleotides at the 5′ end of the first cDNA and the TSO comprises a sequence that hybridizes to the untemplated cytosine nucleotides and the reverse transcriptase extends to generate the TSO complement, which is a compliment of the TSO, at the 5′ end of the first cDNA; eluting the mRNA transcripts from the substrate; contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial barcode sequence complement (SBC′) that is complementary to the spatial barcode sequence (SBC); eluting the second strand; contacting the second strand with an extension mix comprising an extension primer and extending the extension primer using the second strand as a template to generate a double-stranded product while maintaining a single-stranded 3′ region containing the library barcode information, wherein the extension primer hybridizes to a region of the second strand that does not contain the second strand barcode information; contacting the double stranded product with a transposome under conditions to tagment the double stranded product to form a tagmented product comprising a unique molecular identifier (UMI) and PCR adapter; and amplifying the tagmented product using index PCR to generate the library. In some embodiments, the TSO comprises 2-5 guanosines that hybridizes to the untemplated cytosine nucleotides. In some embodiments, the 2-5 guanosines are riboguanosines. In some embodiments, the TSO comprises rGrGrG. In some embodiments, the TSO comprises locked nucleic acids (LNAs).

A method for preparing a RNA sequence library in accordance with the disclosure can include mounting a tissue sample on a substrate comprising a plurality of capture oligonucleotides, wherein the capture oligonucleotides comprise one or more gene-specific capture sequences and library barcode information comprising a spatial barcode sequence (SBC); capturing mRNA transcripts on the substrate with the capture oligonucleotides; contacting the substrate with a first strand synthesis mix comprising a reverse transcriptase and a template switch oligo (TSO) under conditions to generate a first strand comprising a first cDNA complementary to the mRNA transcripts and a TSO complement hybridized to the 5′ end of the first cDNA; eluting the mRNA transcripts from the substrate; contacting the first strand with a blocker oligonucleotide and TSO to hybridize the blocker oligonucleotide and TSO to the first strand, wherein the blocker oligonucleotide and the TSO hybridize such that a gap is present between the blocker oligonucleotide and the TSO; contacting the hybridized first strand with a non-strand displacing polymerase to gap fill the gap and generate a second cDNA; removing the blocker oligonucleotide to form a blocker-free first strand; contacting the blocker-free first strand with a transposome under conditions to tagment the blocker-free first strand to form a tagmented product comprising a unique molecular identifier (UMI) and a PCR adapter; contacting the tagmented product with an extension mix to generate a second strand complementary to the first strand, the second strand comprising second strand barcode information having a spatial barcode sequence complement (SBC′) complementary to the spatial barcode sequence, the second cDNA, the unique molecular identifier, and the PCR adapter; eluting the second strand; and amplifying the second strand using index PCR to generate the library.

A method for preparing a RNA sequence library in accordance with the disclosure can include mounting a tissue sample on a substrate comprising a plurality of capture oligonucleotides, wherein the capture oligonucleotides comprise one or more gene-specific capture sequences and library barcode information comprising a spatial barcode sequence (SBC); capturing mRNA transcripts on the substrate with the capture oligonucleotides; contacting the substrate with a first strand synthesis mix comprising a reverse transcriptase under conditions to generate a first strand comprising a first cDNA complementary to the mRNA transcripts; eluting the mRNA transcripts from the substrate; contacting the first strand with a second strand synthesis mix comprising a random primer and extending the random primer to generate a second strand comprising a second cDNA and a unique molecular identifier (UMI); eluting the second strand; amplifying the second strand to produce a double stranded product; contacting the double stranded product with a transposome under conditions to form a tagmented product; and generating the library by amplifying the tagmented product in a first index PCR to determine the SBC and amplifying the tagmented product in a second index PCR to determine the UMI.

A method for preparing an RNA sequence library in accordance with the disclosure can include mounting a tissue sample on a substrate comprising a plurality of capture oligonucleotides, wherein the capture oligonucleotides comprise a polyT sequence and library barcode information comprising a spatial barcode sequence (SBC); capturing polyadenylated mRNA transcripts on the substrate with the capture oligonucleotides; contacting the substrate with a first strand synthesis mix comprising a reverse transcriptase and a template switch oligonucleotide (TSO) under conditions to generate a first strand comprising a first cDNA complementary to the polyadenylated mRNA transcripts, and a TSO complement hybridized to the 5′ end of the first cDNA, wherein the reverse transcriptase incorporates untemplated cytosine nucleotides at the 5′ end of the first cDNA and the TSO comprises a sequence that hybridizes to the untemplated cytosine nucleotides and the reverse transcriptase extends to generate a compliment of the TSO (the TSO complement) at the 5′ end of the first cDNA; eluting the polyadenylated mRNA transcripts from the substrate; contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial barcode sequence complement (SBC′) complementary to the spatial barcode sequence (SBC); eluting the second strand; contacting the second strand with a Poly-TVN extension mix comprising a Poly-TVN primer (e.g., a poly(T) primer with VN anchor at the 3′ end, wherein V is G, A, or C and N is any nucleotide) and extending the Poly-TVN primer using the second strand as a template to generate a double-stranded product while maintaining a single-stranded 3′ region containing the library barcode information; contacting the double stranded product with a transposome under conditions to tagment the double stranded product to form a tagmented product comprising a unique molecular identifier (UMI) and PCR adapter; and amplifying the tagmented product using index PCR to generate the library. Extension of the second strand can, in some embodiments, include an incubation time with the Poly-TVN extension mix of less than 2 hours, for examples, 15 minutes to 60 minutes. In some embodiments, the TSO comprises 2-5 guanosines that hybridize to the untemplated cytosine nucleotides. In some embodiments, the 2-5 guanosines are riboguanosines. In some embodiments, the TSO comprises rGrGrG, In some embodiments, the TSO comprises locked nucleic acids (LNAs).

A method for preparing a RNA sequence library in accordance with the disclosure can include mounting a tissue sample on a substrate comprising a plurality of capture oligonucleotides, wherein the capture oligonucleotides comprise a polyT sequence and library barcode information comprising a spatial barcode sequence (SBC); capturing polyadenylated mRNA transcripts on the substrate with the capture oligonucleotides; contacting the substrate with a first strand synthesis mix comprising a reverse transcriptase and a template switch oligo (TSO) under conditions to generate a first strand comprising a first cDNA complementary to the polyadenylated mRNA transcripts and a TSO complement hybridized to the 5′ end of the first cDNA; eluting the polyadenylated mRNA transcripts from the substrate; contacting the first strand with a blocker oligonucleotide and TSO to hybridize the blocker oligonucleotide and TSO to the first strand, wherein the blocker oligonucleotide and the TSO hybridize such that a gap is present between the blocker oligonucleotide and the TSO; contacting the hybridized first strand with a non-strand displacing polymerase to gap fill the gap and generate a second cDNA; removing the blocker oligonucleotide to form a blocker-free first strand; contacting the blocker-free first strand with a transposome under conditions to tagment the blocker-free first strand to form a tagmented product comprising a unique molecular identifier (UMI) and a PCR adapter; contacting the tagmented product with an extension mix to generate a second strand complementary to the first strand, the second strand comprising second strand barcode information having a spatial barcode sequence complement (SBC′) complementary to the spatial barcode sequence, the second cDNA, the unique molecular identifier, and the PCR adapter; eluting the second strand; and amplifying the second strand using index PCR to generate the library. In embodiments, the blocker oligonucleotide can be a 3′-blocked SBS12′-PolyA oligonucleotide.

A method for preparing a RNA sequence library in accordance with the disclosure can include mounting a tissue sample on a substrate comprising a plurality of capture oligonucleotides, wherein the capture oligonucleotides comprise a polyT sequence and library barcode information comprising a spatial barcode sequence (SBC); capturing polyadenylated mRNA transcripts on the substrate with the capture oligonucleotides; contacting the substrate with a first strand synthesis mix comprising a reverse transcriptase under conditions to generate a first strand comprising a first cDNA complementary to the polyadenylated mRNA transcripts; eluting the polyadenylated mRNA transcripts from the substrate; contacting the first strand with a second strand synthesis mix comprising a random primer to generate a second strand comprising a second cDNA and a unique molecular identifier (UMI); eluting the second strand; amplifying the second strand to produce a double stranded product; contacting the double stranded product with a transposome under conditions to form a tagmented product; and generating the library by amplifying the tagmented product in a first index PCR to determine the SBC and amplifying the tagmented product in a second index PCR to determine the UMI.

In one aspect, the disclosure provides a method for preparing a spatially barcoded RNA library from a tissue sample comprising,
a) contacting the tissue sample with a plurality of capture oligonucleotides immobilized on a solid substrate and capable of hybridizing with RNA in the tissue sample, wherein the capture oligonucleotides comprise a capture nucleotide sequence, a spatial barcode sequence (SBC), and adapter sequences, wherein RNA transcripts are captured by the capture nucleotide sequence of the plurality of capture oligonucleotides;
b) contacting the RNA transcripts with a first strand synthesis mix comprising a reverse transcriptase (RT) and a template switch oligonucleotide (TSO) encoding a first adapter sequence under conditions to generate a first strand cDNA comprising
a first strand cDNA complementary to the RNA transcripts and a TSO hybridized to a 3′ end of the first strand cDNA, wherein the reverse transcriptase incorporates untemplated cytosine nucleotides at the 3′ end of the first strand cDNA and the TSO comprising a first adapter sequence is appended to the 3′ end of the first strand cDNA and the reverse transcriptase extends to generate a TSO complement; wherein the contacting generates a mixture of template switched molecules and non-template switched molecules, wherein the non-template switched molecules lack the complement to the first adapter sequence;
c) contacting the mixture with a plurality of oligo ligation blockers comprising the first adapter sequence and capable of hybridizing with the template switched molecule 3′ end comprising the complement to the first adapter sequence;
d) carrying out a single-stranded ligation step comprising hybridizing a splint adapter to the non-template-switched molecules, wherein the splint adapter comprises i) a single-stranded splint sequence comprising a random base sequence (NX) and ii) a double-stranded first adapter sequence comprising hybridized first adapter and complement to the first adapter sequences, wherein the complement to the first adapter sequence 5′ end contains a phosphate group for ligation to the captured non-template switched molecules 3′OH end, and ligating the 5′ end of the splint adapter complement to the 3′OH end of the non-template switched molecules;
e) removing the ligation blockers and the splint sequence and first adapter from the ligated molecules.

In another aspect, the disclosure provides a method for preparing a spatially barcoded RNA library from a tissue sample comprising,
a) contacting the tissue sample with a plurality of capture oligonucleotides immobilized on a solid substrate and capable of hybridizing with RNA in the tissue sample, wherein the capture oligonucleotides comprise a capture nucleotide sequence, a spatial barcode sequence (SBC), and adapter sequences, wherein RNA transcripts are captured by the capture nucleotide sequence of the plurality of capture oligonucleotides;
b) contacting the RNA transcripts with a first strand synthesis mix comprising a reverse transcriptase (RT) and a template switch oligonucleotide (TSO) encoding a first adapter sequence under conditions to generate a first strand cDNA comprising
a first strand cDNA complementary to the RNA transcripts and a TSO appended to a 3′ end of the first strand cDNA, wherein the reverse transcriptase incorporates untemplated cytosine nucleotides at the 3′ end of the first strand cDNA and the TSO comprising a first adapter sequence is appended to the 3′ end of the first strand cDNA and the reverse transcriptase extends to generate a TSO complement; wherein the contacting generates a mixture of template switched molecules and non-template switched molecules, wherein the non-template switched molecules lack the complement to the first adapter sequence;
c) contacting the mixture with a plurality of oligo ligation blockers comprising the first adapter sequence and capable of hybridizing with the template switched molecule 3′ end comprising the complement to the first adapter sequence and a plurality of complementary oligo blockers comprising nucleotide sequences complementary to all or part of the capture nucleotide sequence and a fixed sequence in the capture oligonucleotide to generate a double-stranded 3′ terminus of the capture oligonucleotide;
d) carrying out a single-stranded ligation step comprising hybridizing a splint adapter to the non-template-switched molecules, wherein the splint adapter comprises i) a single-stranded splint sequence comprising a random base sequence (NX) and ii) a double-stranded partial first adapter sequence comprising hybridized first adapter and complement to the first adapter sequences, wherein the complement to the first adapter sequence 5′ end contains a phosphate group for ligation to the captured non-template switched molecules 3′OH end, and ligating the 5′ end of the splint adapter complement to the 3′OH end of the non-template switched molecules;
f) removing the ligation blockers and the splint strand of the adapter.

It is contemplated that the oligo ligation blockers block the template switched molecule and/or the capture nucleotide, e.g., as described in FIG. 12.

In various embodiments, the mixture of template switched molecules and non-template switched molecules is contacted with an exonuclease. In various embodiments, the exonuclease is DNA exonuclease I or RNAse H.

In various embodiments, the NX sequence has a blocking group at the NX sequence 5′ end. In various embodiments, the hybridized first adapter and complement to the first adapter sequences comprise blocking groups at ends of both the first adapter and complement to the first adapter sequences furthest from the splint sequence.

In various embodiments, the methods further comprise after the exonuclease, contacting the mixture with an alkaline solution.

In various embodiments, the methods further comprise, after the removing step, amplifying the template switched and non template switched molecules by contacting the mixture with a second strand synthesis mix comprising a single first adapter primer and extending the first adapter primer using the first strand cDNA or complement thereof as a template to generate a second strand cDNA complementary to the first strand or complement thereof, the second strand cDNA comprising a second cDNA complementary to the first strand cDNA, and second strand barcode information comprising a spatial barcode sequence complement (SBC′) that is complementary to the spatial barcode sequence (SBC) in the capture oligonucleotide.

In various embodiments, the first adapter primer comprises a molecular identifier (SMI) sequence. In various embodiments, the molecular identifier of the first adapter primer is incorporated during second strand cDNA synthesis. In various embodiments, the SMI is a UMI. In various embodiments, if the first strand cDNA comprises a SMI, it is contemplated that the SMI on the first adapter primer is not the same as the SMI in the first strand cDNA. See also FIG. 22.

In various embodiments, the first adapter primer is a full length primer or partial primer.

In various embodiments, the methods further comprise eluting the amplified first strand and/or second strand cDNA molecules from the substrate and generating a spatially barcoded RNA library from the eluted molecules using a library prep kit.

In various embodiments, the ligated molecules of step (d) further comprise a cleavage sequence. In various embodiments, the capture oligo further comprises a cleavage site. In various embodiments, the cleavage site is 5′ to the clustering sequence (e.g., P7).

In various embodiments, removing the ligation blockers, splint sequence and first adapter from the ligated molecules is carried out off the substrate. In various embodiments, removing the ligation blockers is carried out off the substrate, amplifying the template switched and non template switched molecules, eluting the second strand cDNA and generating a spatially barcoded library are performed in solution.

In various embodiments, the amplifying step is carried out on the substrate. In various embodiments, the amplified first strand and/or second strand further comprise a cleavage sequence. In various embodiments, the amplified molecules contain a cleavage sequence, and the amplified molecules are released form the substrate via the cleavage sequence.

In various embodiments, when the amplified molecules are cleaved from the substrate, eluting the second strand cDNA and generating a spatially barcoded library are performed in solution.

In various embodiments, the methods optionally comprise mounting the tissue sample on a substrate comprising the plurality of capture oligonucleotides prior to contacting the tissue with the plurality of capture oligonucleotides. In various embodiments, the capture nucleotide sequence is a poly-T sequence, a poly-A sequence, a gene-specific capture sequence, or a universal capture sequence. In various embodiments, the universal capture sequence is a random nucleotide sequence or a non-self complementary semi-random sequence.

In various embodiments, the capture oligonucleotide comprises; a 5′ clustering sequence, a randomized spatial barcode (SBC), a full-length second adapter sequence (2 FL), a molecular identifier (MI), a fixed sequence (FS), and a poly T capture sequence with a 3′ VN terminus (polyTVN).

In various embodiments, the first adapter sequence is a read 1 (Rd1) sequence. In various embodiments, the first adapter sequence is a read 1 (Rd1) sequence, and the second adapter is a read 2 (Rd2) sequence. In various embodiments, the first adapter is a partial adapter sequence.

In various embodiments, the molecular identifier is a unique molecular identifier, an endogenous molecular identifier, an exogenous molecular identifier, or a virtual molecular identifier.

In various embodiments, the ligation step comprises enzymatic ligation of the splint adapter to the non-template switched molecule. In various embodiments, the enzymatic ligation is by T4 ligase, other DNA ligase, or thermostable 5′ App DNA/RNA ligase-mediated ligation with a synthesized pre-adenylated single-stranded oligo adapter.

In various embodiments, the ligation step comprises chemical ligation of the splint adapter to the non-template switched molecule. In various embodiments, the chemical ligation is carried out using click-chemistry-mediated ligation wherein 3′ azido termini are joined to synthesized 5′ alkyne single-stranded oligos or 1-ethyl-3-(3-dimethylaminopropyl) carbodiimide (EDC)-mediated ligation wherein cDNAs with 3′ phosphate groups are ligated to splinted adapters with 5′ hydroxyl termini. In various embodiments, 3′ azido-ddNTPs or 3′ Phos-dATPs are incorporated onto the Rd1 adapter during first strand cDNA synthesis.

In various embodiments, the splint adapter random sequence comprises between 6 and 10 nucleotides. In various embodiments, the splint adapter random sequence comprises 6, 7, 8, 9, or 10 nucleotides. In various embodiments, the splint adapter random sequence comprises 7 nucleotides.

In various embodiments, the splint adapter comprises blocking groups at both 3′ ends and the 5′ end of the splint and a ligation blocking group at the 5′ end of the adapter strand that is complementary to the splinted strand. In various embodiments, the ligation blocker group is a phosphate, 3′ dideoxy C, 3′ inverted dT, 3′ carbon spacer, 3′ amino or 3′ biotin. In various embodiments, the ligation blocker group is a phosphate.

In various embodiments, the ligation blocker and splint adapter are removed by alkaline treatment. In various embodiments, the alkaline treatment comprises either 0.08 M KOH or 0.1 N NaOH for five minutes at room temperature.

In various embodiments, the 5′ clustering sequence comprises a P7 sequence.

In various embodiments, the capture oligonucleotide further comprises a randomer, a semi-random sequence, or a target-specific probe.

In various embodiments, the sequence that hybridizes to the untemplated cytosine nucleotides comprises 2-5 guanosines. In various embodiments, the guanosines are riboguanosines, modified nucleic acids or locked nucleic acids (LNA). In various embodiments, the sequence is rGrGrG.

In various embodiments, the polyT sequence is between 20-30 nucleotides.

In various embodiments, the SBC is a randomer. In various embodiments, the SBC is between 20 and 30 nucleotides.

In various embodiments, the capture oligonucleotide comprises at least 8 deoxythymidine residues. In various embodiments, the capture oligonucleotide is between 8 to 80 nucleotides.

In various embodiments, the capture oligonucleotide comprises a plurality of different target-specific RNA capture probe sequences. In various embodiments, the target-specific probes comprise at least 8 nucleotides complementary to a nucleotide sequence of a target RNA. In various embodiments, the RNA capture oligonucleotide is between 8 to 80 nucleotides, between 10 to 70 nucleotides, between 10 to 60 nucleotides, between 10 to 50 nucleotides, between 10 to 40 nucleotides, between 10 to 30 nucleotides, between 10 to 20 nucleotides, between 20 to 80 nucleotides, between 20 to 70 nucleotides, between 20 to 60 nucleotides, between 20 to 50 nucleotides, between 20 to 40 nucleotides, or is 8, 9, 10, 11, 12, 13, 14, 15, 16, 17, 18, 19, 20, 21, 22, 23, 24, 25, 26, 27, 28, 29, 30, 40, 50, 60, 70 or 80 nucleotides.

In various embodiments, the capture oligonucleotide comprises a P7 anchor sequence, a spatial barcode and a sequence that hybridizes with a splint oligonucleotide.

In various embodiments, one or more of a first clustering sequence, an index sequence, and/or a Read 2 sequence are added during or prior to second strand synthesis.

In various embodiments, the methods further comprise, prior to the step of capturing RNA from the tissue sample, the step of performing end repair of the RNA with polynucleotide kinase. In various embodiments, the methods further comprise, prior to the step of capturing RNA from the tissue sample, the step of performing in situ polyadenylation with polyadenylate polymerase. In various embodiments, the methods further comprise, prior to the step of capturing RNA from the tissue sample, the steps of performing end repair of the RNA with polynucleotide kinase followed by performing in situ polyadenylation with polyadenylate polymerase.

In various embodiments, the RNA comprises ribosomal RNA (rRNA), messenger RNA (mRNA), non-coding RNA (ncRNA), small nuclear RNA (snRNA), small nucleolar RNA (snoRNA), and/or microRNA (miRNA).

In various embodiments, the tissue sample is formalin-fixed paraffin embedded (FFPE) tissue or fresh frozen (FF) tissue.

In various embodiments, removing the RNA is carried out by melting the RNA or digestion with an RNase.

In various embodiments, the tissue sample is permeabilized prior to contacting the tissue sample with a plurality of capture oligonucleotides. In various embodiments, the tissue sample is treated with one or more blocking reagents prior to contacting the tissue sample with a plurality of capture oligonucleotides. In various embodiments, the tissue sample is permeabilized and treated with one or more blocking reagents prior to contacting the tissue sample with a plurality of capture oligonucleotides.

In various embodiments, the tissue is removed from the sample by enzymatic degradation. In various embodiments, the tissue removal is carried out before the RNA is removed from the tissue. In various embodiments, the tissue is removed via degradation with proteinase K, e.g., at 37° C. for 40 minutes.

In various embodiments, the substrate is a bead, a bead array, a spotted array, a substrate comprising a plurality of wells, a flow cell, clustered particles arranged on a surface of a chip, a film, or a plate. In various embodiments, the substrate comprises a plurality of nanowells or microwells.

In various embodiments, the substrate or surface of the substrate comprises a material selected from glass, silicon, poly-L-lysine coated materials, nitro-cellulose, polystyrene, cyclic olefin copolymers (COCs), cyclic olefin polymers (COPs), poly-acrylamide, polypropylene, polyethylene, or polycarbonate.

In various embodiments, the RNA library is an mRNA library.

In various embodiments, the methods further comprise indexing and sequencing the second stand cDNA comprising,
performing PCR on the second strand cDNA to yield a PCR template representative of one or more RNA transcripts in the tissue sample;
eluting the PCR template; and
carrying out an indexing PCR to generate a double stranded PCR product comprising the first strand PCR product and a second strand complementary to the first strand PCR product.

In various embodiments, the methods further comprise sequencing the PCR product and determining the location of the RNA transcript in the tissue based on the spatial barcode.

In various embodiments, the double stranded PCR product comprises a second clustering sequence on the second strand complementary to the first strand PCR product and, optionally, an index sequence. In various embodiments, the double stranded PCR product are further processed by tagmentation to generate a spatial transcriptomics library.

In various embodiments, the tagmentation comprises on substrate tagmentation.

In various embodiments, the tagmentation comprises contacting the double stranded product with the transposome and a carrier genomic DNA (gDNA).

In various embodiments, the methods further comprise determining spatial locations of the spatial barcodes of the plurality of capture oligonucleotide molecules prior to the step of contacting the tissue with the substrate.

In various embodiments, the methods further comprise sequencing at least a portion of the spatially barcoded first strand cDNA or copies thereof to determine the spatial barcode sequence for each molecule. In various embodiments, the spatially barcoded first strand cDNA is sequenced in situ.

In various embodiments, the methods further comprise determining the spatial location of one or more of the spatially barcoded first strand cDNA or copies thereof by correlating the spatial barcode sequences of the spatially barcoded first strand cDNA or copies thereof with the spatial locations of the capture oligonucleotide molecules on the substrate containing corresponding spatial barcode sequences. In various embodiments, the methods further comprise recovering the spatially barcoded first strand cDNA and amplifying the first strand cDNA to generate cDNA libraries.

In various embodiments, the spatially barcoded first strand cDNA is recovered by contacting the spatially barcoded first strand cDNAs on the substrate with a DNA polymerase and one or more primers to generate spatially barcoded second strand cDNAs complementary to the spatially barcoded first strand cDNAs and removing the spatially barcoded second strand cDNAs from the substrate. In various embodiments, the one or more primers each comprise a random priming sequence. In various embodiments, the random priming sequences comprises nine random nucleotides.

In various embodiments, the spatially barcoded second strand cDNAs each comprise a unique molecular identifier (UMI), wherein the UMI comprises an intrinsic sequence and an extrinsic sequence, wherein the extrinsic sequence is a sequence complementary to the random priming sequence used to generate the second strand cDNA, and wherein the intrinsic sequence is a sequence complementary to the first strand cDNA template sequence used to generate the second strand cDNA.

In various embodiments, the one or more primers each comprise a molecular identifier barcode. In various embodiments, the one or more primers each comprise a UMI barcode.

In various embodiments, the spatially barcoded second strand cDNAs are removed from the substrate by chemical or physical dehybridization.

In various embodiments, the capture oligonucleotide comprises an anchor sequence comprising a cleavage site that anchors the capture oligonucleotide to the substrate, and hybrids of the spatially barcoded first and second strand cDNAs are removed from the substrate by enzymatic cleavage at the cleavage site. In various embodiments, the cleavage site is a binding site for a restriction endonuclease.

In various embodiments, the methods further comprise sequencing at least a portion of the cDNA libraries to determine the spatial barcode sequence for each molecule.

In various embodiments, the methods further comprise determining the spatial location of one or more cDNA molecules by correlating the spatial barcode sequences of the one or more cDNA molecules with the spatial locations of the surface oligonucleotide molecules on the substrate containing corresponding spatial barcode sequences.

In various embodiments, RNA expression in a single cell within the tissue is determined. In various embodiments, RNA expression in a subcellular component within a single cell is determined. In various embodiments, the subcellular component is a nucleus, mitochondria, ribosomes or cytoplasm.

The disclosure also contemplates a kit comprising
a) a solid substrate comprising capture oligonucleotides immobilized on the solid substrate, wherein the capture oligonucleotides comprise a capture nucleotide sequence, a spatial barcode sequence (SBC), and adapter sequences;
b) a reverse transcriptase (RT) and a template switch oligonucleotide (TSO) encoding a first adapter sequence); and
c) a splint adapter, wherein the splint adapter comprises
   i) a single-stranded splint sequence comprising a random base sequence (NX) having a blocking group at the NX sequence 5′ end; and
   ii) a double-stranded first adapter sequence comprising hybridized first adapter sequence and complementary to first adapter sequences, optionally wherein the hybridized first adapter sequence and complementary to first adapter sequences comprise blocking groups at the 5′ ends of the first adapter and the 3′ end of the complement to first adapter sequences.

The present disclosure also provides, in various aspects, methods of RNA-seq library preparation that utilize on-surface enzymatic extension and strand termination to normalize library size. In various embodiments, transcripts are captured on a barcoded surface and reverse transcriptase is initiated with nucleotides, such as dUTP or ddNTPs to allow for decreased fragment size. Such methods can be applied to various library prep methods where shorter library fragments are desired.

Accordingly, in some aspects the disclosure provides a method of preparing an immobilized library of target nucleic acids of a biological sample, comprising: (a) providing a surface comprising a plurality of capture oligonucleotides immobilized thereon, wherein one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence; (b) contacting the biological sample with the surface, the contacting resulting in hybridization of the target nucleic acids of the biological sample to the capture nucleotide sequence of the plurality of capture oligonucleotides to form hybridized capture oligonucleotides; (c) extending the capture nucleotide sequence of the hybridized capture oligonucleotides to form first complementary strands of the target nucleic acids, wherein the extending is in the presence of an extension termination moiety, and wherein the extension termination moiety is an allyl-T or a deoxyuridine triphosphate (dUTP), thereby preparing the immobilized library of target nucleic acids. In some embodiments, the method further comprises (d) contacting the surface with an exonuclease; (e) hybridizing a plurality of oligonucleotide primers to the first complementary strands, wherein each of the plurality of oligonucleotide primers comprises, from 5′ to 3′: (i) an adapter nucleotide sequence; and (ii) a random nucleotide sequence; (f) extending the plurality of oligonucleotide primers, thereby generating one or more second complementary strands comprising the adapter nucleotide sequence at a terminus. In further embodiments, the method further comprises (g) removing the one or more second complementary strands from the surface and amplifying the one or more second complementary strands. In some embodiments, step (g) is performed in the presence of an Exclusion Amplification (ExAmp) mix, wherein the ExAmp mix comprises a primer comprising the clustering primer sequence. In some embodiments, one or more of the plurality of capture oligonucleotides is immobilized on the surface through a cleavage site. In further embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In some embodiments, the cleavage site is cleaved after step (c). In further embodiments, one or more of the plurality of capture oligonucleotides is immobilized on the surface through a cleavage site. In some embodiments, the cleavage site is an enzymatic cleavage site. In further embodiments, the enzymatic cleavage site comprises a restriction enzyme site, a uracil, an 8-oxoguanine, or a combination thereof. In some embodiments, the cleavage site is a chemical cleavage site. In further embodiments, the cleavage site is cleaved after step (f). In various embodiments, the extension termination moiety is a deoxyuridine triphosphate (dUTP) and the method further comprises contacting the surface with a uracil-DNA glycosylase (UDG). In some embodiments, the extension termination moiety is an allyl-T and wherein the method further comprises contacting the surface with a universal cleavage mix (UCM). In some embodiments, the extension termination moiety is a deoxyuridine triphosphate (dUTP) and the method further comprises contacting the surface with a uracil-DNA glycosylase (UDG). In some embodiments, the extension termination moiety is an allyl-T and wherein the method further comprises contacting the surface with a universal cleavage mix (UCM) prior to step (e).

In some aspects, the disclosure provides a method of preparing an immobilized library of target nucleic acids of a biological sample, comprising: (a) providing a surface comprising a plurality of capture oligonucleotides immobilized thereon, wherein one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence; (b) contacting the biological sample with the surface, the contacting resulting in hybridization of the target nucleic acids of the biological sample to the capture nucleotide sequence of the plurality of capture oligonucleotides to form hybridized capture oligonucleotides; (c) extending the capture nucleotide sequence of the hybridized capture oligonucleotides to form first complementary strands of the target nucleic acids, wherein the extending is in the presence of an extension termination moiety, and wherein the extension termination moiety is a dideoxynucleoside triphosphate (ddNTP), thereby preparing the immobilized library of target nucleic acids. In some embodiments, the method further comprises (d) contacting the surface with an exonuclease; (e) hybridizing a plurality of oligonucleotide primers to the first complementary strands, wherein each of the plurality of oligonucleotide primers comprises, from 5′ to 3′: (i) an adapter nucleotide sequence; and (ii) a random nucleotide sequence; (f) extending the plurality of oligonucleotide primers, thereby generating one or more second complementary strands comprising the adapter nucleotide sequence at a terminus. In some embodiments, the method further comprises (g) removing the one or more second complementary strands from the surface and amplifying the one or more second complementary strands. In some embodiments, step (g) is performed in the presence of an Exclusion Amplification (ExAmp) mix, wherein the ExAmp mix comprises a primer comprising the first clustering primer sequence. In some embodiments, one or more of the plurality of capture oligonucleotides is immobilized on the surface through a cleavage site. In further embodiments, the cleavage site is an enzymatic cleavage site. In some embodiments, the enzymatic cleavage site comprises a restriction enzyme site, a uracil, an 8-oxoguanine, or a combination thereof. In further embodiments, the cleavage site is a chemical cleavage site. In some embodiments, the cleavage site is cleaved after step (c). In various embodiments, one or more of the plurality of capture oligonucleotides is immobilized on the surface through a cleavage site. In some embodiments, the cleavage site is an enzymatic cleavage site. In further embodiments, the enzymatic cleavage site comprises a restriction enzyme site, a uracil, an 8-oxoguanine, or a combination thereof. In further embodiments, the cleavage site is a chemical cleavage site. In some embodiments, the cleavage site is cleaved after step (f). In some embodiments, the ddNTP comprises a first click chemistry handle. In some embodiments, the method further comprises, after step (c), contacting the surface with an adapter oligonucleotide comprising a second click chemistry handle capable of crosslinking to the first click chemistry handle, thereby ligating the adapter oligonucleotide to the first complementary strands. In some embodiments, the adapter oligonucleotide further comprises a second sequencing primer sequence. In further embodiments, the first click chemistry handle is an azide, a tetrazine, a strained alkene, or an alkyne. In still further embodiments, the second click chemistry handle is an azide, a tetrazine, a strained alkene, or an alkyne.

In further aspects, the disclosure provides a method of preparing an immobilized library of target nucleic acids of a biological sample, comprising: (a) providing a surface comprising a plurality of capture oligonucleotides immobilized thereon, wherein one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence; (b) contacting the biological sample with the surface, the contacting resulting in hybridization of the target nucleic acids of the biological sample to the capture nucleotide sequence of the plurality of capture oligonucleotides to form hybridized capture oligonucleotides; (c) extending the capture nucleotide sequence of the hybridized capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate, thereby preparing the immobilized library of target nucleic acids. In some embodiments, the method further comprises (d) contacting the surface with an exonuclease; and (e) contacting the surface with a ligase enzyme, thereby ligating an adapter oligonucleotide to the first complementary strands, wherein the adapter oligonucleotide comprises, from 5′ to 3′: (i) an adapter nucleotide sequence; and (ii) a random nucleotide sequence, and wherein the adapter oligonucleotide further comprises a second oligonucleotide that is hybridized to the adapter nucleotide sequence. In some embodiments, the adapter nucleotide sequence comprises a second sequencing primer sequence. In further embodiments, the ligating occurs through a splinted ligation of the adapter oligonucleotide to the first complementary strands. In further embodiments, the ligase enzyme is a T4 DNA ligase. In some embodiments, the method further comprises (f) extending the adapter oligonucleotide, thereby generating one or more second complementary strands. In some embodiments, the method further comprises (d) contacting the surface with an exonuclease; and (e) contacting the surface with a ligase enzyme, thereby ligating an adapter oligonucleotide to the first complementary strands, wherein the adapter oligonucleotide comprises, from 5′ to 3′: (i) a random nucleotide sequence; and (ii) an adapter nucleotide sequence. In some embodiments, the adapter nucleotide sequence comprises a second sequencing primer sequence. In some embodiments, the ligating occurs through a single-stranded DNA ligation of the adapter oligonucleotide to the first complementary strands. In further embodiments, the ligase enzyme is a DNA/RNA ligase. In some embodiments the method further comprises (f) extending the adapter oligonucleotide, thereby generating one or more second complementary strands. In further embodiments, the method further comprises (g) removing the one or more second complementary strands from the surface and amplifying the one or more second complementary strands. In some embodiments, step (g) is performed in the presence of an Exclusion Amplification (ExAmp) mix. In further embodiments, one or more of the plurality of capture oligonucleotides is immobilized on the surface through a cleavage site. In some embodiments, the cleavage site is an enzymatic cleavage site. In further embodiments, the enzymatic cleavage site comprises a restriction enzyme site, a uracil, an 8-oxoguanine, or a combination thereof. In some embodiments, the cleavage site is a chemical cleavage site. In further embodiments, the cleavage site is cleaved after step (c). In some embodiments, one or more of the plurality of capture oligonucleotides is immobilized on the surface through a cleavage site. In further embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In further embodiments, the cleavage site is cleaved after step (e).

In further aspects, the disclosure provides a method of preparing an immobilized library of target nucleic acids of a biological sample, comprising: (a) providing a surface comprising a plurality of capture oligonucleotides immobilized thereon, wherein one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence; (b) contacting the biological sample with the surface, the contacting resulting in hybridization of the target nucleic acids of the biological sample to the capture nucleotide sequence of the plurality of capture oligonucleotides to form hybridized capture oligonucleotides; (c) extending the capture nucleotide sequence of the hybridized capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate or a dideoxynucleoside triphosphate (ddNTP) comprising a first click chemistry handle, thereby preparing the immobilized library of target nucleic acids. In some embodiments, the extension termination moiety is the deoxynucleoside triphosphate (dNTP) comprising a 3′ phosphate. In some embodiments, the method further comprises (d) chemically ligating an adapter oligonucleotide to the first complementary strands through a crosslinking group, wherein the adapter oligonucleotide comprises, from 5′ to 3′: (i) an adapter nucleotide sequence; and (ii) a random nucleotide sequence, and wherein the adapter oligonucleotide further comprises a second oligonucleotide that is hybridized to the adapter nucleotide sequence. In some embodiments, the adapter nucleotide sequence comprises a second sequencing primer sequence. In various embodiments, the crosslinking group is a carboxyl-to-amine reactive group, a BCN-azide reactive group, a DBCO-azide reactive group, a Tetrazine-TCO reactive group, or a combination thereof. In some embodiments, the extension termination moiety is the dideoxynucleoside triphosphate (ddNTP) comprising the first click chemistry handle. In some embodiments, the method further comprises (d) ligating an adapter oligonucleotide to the first complementary strands through click chemistry, wherein the adapter oligonucleotide comprises, from 5′ to 3′: (i) an adapter nucleotide sequence; and (ii) a random nucleotide sequence, and wherein the adapter oligonucleotide further comprises a second oligonucleotide that is hybridized to the sequencing primer sequence, wherein the second oligonucleotide comprises a second click chemistry handle. In some embodiments, the adapter nucleotide sequence comprises a second sequencing primer sequence. In various embodiments, the first click chemistry handle is an azide, a tetrazine, a strained alkene, or an alkyne. In some embodiments, the second click chemistry handle is an azide, a tetrazine, a strained alkene, or an alkyne. In some embodiments, the method further comprises (e) extending the adapter oligonucleotide, thereby generating one or more second complementary strands. In some embodiments, the method further comprises (f) removing the one or more second complementary strands from the surface and amplifying the one or more second complementary strands. In some embodiments, step (f) is performed in the presence of an Exclusion Amplification (ExAmp) mix. In some embodiments, one or more of the plurality of capture oligonucleotides is immobilized on the surface through a cleavage site. In some embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In further embodiments, the cleavage site is cleaved after step (c). In some embodiments, one or more of the plurality of capture oligonucleotides is immobilized on the surface through a cleavage site. In some embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In some embodiments, the cleavage site is cleaved after step (d).

In some embodiments, a method of the disclosure further comprises removing the target nucleic acids from the surface after step (c). In some embodiments, a method of the disclosure further comprises removing the biological sample from the surface after step (d). In various embodiments, each of the plurality of capture oligonucleotides comprises the same capture nucleotide sequence. In some embodiments, the plurality of capture oligonucleotides comprises multiple, different capture nucleotide sequences. In further embodiments, the multiple, different capture nucleotide sequences comprise one or more gene-specific capture sequences, one or more universal capture sequences, or a combination thereof. In various embodiments, the capture nucleotide sequence is a poly-T sequence, a poly-A sequence, a gene-specific capture sequence, or a universal capture sequence. In some embodiments, the universal capture sequence is a random nucleotide sequence or a non-self complementary semi-random sequence. In various embodiments, the target nucleic acids are mRNA, gDNA, RNA, tRNA, or a combination thereof. In some embodiments, the target nucleic acids are RNA, mRNA, or a combination thereof. In some embodiments, the target nucleic acids are cDNA generated from RNA by reverse transcription, wherein a homopolymer capture sequence (e.g., a poly-A sequence) is added to the 3′ end of the cDNA (e.g., by ligation or by a terminal transferase enzyme such as terminal deoxynucleotidyl transferase (TdT)). In some embodiments, the extending of the capture nucleotide sequence in step (c) is carried out using a reverse transcriptase. In some embodiments, the target nucleic acids are polyadenylated prior to hybridization of the target nucleic acids to the capture nucleotide sequences. In various embodiments, the target nucleic acids are polyadenylated using a poly(A) polymerase. In further embodiments, the target nucleic acids are polyadenylated using chemical ligation or enzymatic ligation. In some embodiments, the amplifying comprises addition of a second clustering primer sequence to the one or more second complementary strands. In some embodiments, the amplifying further comprises addition of an indexing sequence. In some embodiments, the amplifying comprises index PCR during which a first primer hybridizes to the first clustering primer sequence and a second primer hybridizes to the adapter nucleotide sequence, wherein the second primer comprises the second clustering primer sequence. In some embodiments, the second primer further comprises the indexing sequence.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “various embodiments”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1A is schematic illustration of a method of preparing an RNA sequence library in accordance with the disclosure.

FIG. 1B is a schematic illustration of the addition of the TSO complement during first strand synthesis in a method in accordance with the disclosure. Figure is adapted from Integrated DNA Technologies, “Use of Template Switching Oligos (TS Oligos, TSOs) for efficient cDNA library construction” Education webpage.

FIG. 2 is a schematic illustration of the read sequence for the method of FIG. 1.

FIG. 3 is a process flow diagram for a method of preparing an RNA sequence library in accordance with the disclosure.

FIG. 4A is a graph showing Tapestation data for methods of the disclosure comparing the purified starting input 2.5×SPRI purification vs. 0.7×SPRI purification.

FIG. 4B is a graph showing Tapestation data showing the fragment sizes for the two tagmentation dilutions: high input P7/TSO amplicon (2.5 ng) vs. a low input amplicon (100 pg).

FIG. 5A is a schematic illustration of on-bead tagmentation testing 500 μg, 100 pg and 10 pg P7/TSO amplicon in methods in accordance with the disclosure.

FIG. 5B is a graph showing library fragment size for the testing in FIG. 5A.

FIG. 6 is a schematic illustration of testing conditions for evaluating effect of the amount of transposase and purification vs no purification.

FIG. 7 is a graph showing an alignment distribution for a method in accordance with the disclosure as compared to the commercially available Visium method.

FIG. 8 is a graph showing transcript coverage of a method in accordance with the disclosure.

FIGS. 9A and 9B are schematic illustrations of (A) in-solution and (B) on-surface methods in accordance with the disclosure.

FIG. 10 is a schematic illustration comparing methods in accordance with the disclosure with and without the use of a template switch oligonucleotide.

FIG. 11A shows a schematic for generating a transposition amplified TSO library.

FIG. 11B shows base composition plots of Read 1 in samples from the TSP library. FIG. 11C shows library concentration determined by Screentape analysis. FIG. 11D shows the # of UMIs per 5M input raw reads.

FIG. 12 is a workflow showing steps in a single stranded RNA library preparation which combines on-surface template-switching and single-stranded enzymatic ligation (TSO-LIG) to convert tissue RNAs into spatially barcoded libraries.

FIG. 13 is a workflow showing both enzymatic and chemical methods for converting second adapter-containing synthesized cDNA to libraries via single-stranded ligation of a first adapter sequence to the 3′ terminus of the first strand cDNA.

FIG. 14 shows sensitivities for template switching (TSO), single-stranded splinted ligation (LIG) and both methods combined (TSO+LIG). Results are shown as sensitivity UMI per bin 100 adapter, fold change relative to TSO. Sensitivity was calculated as median UMIs detected per 100×100 μm and then normalized relative to the TSO condition. Error bars are STDEV from 4 tissue sections.

FIG. 15A-15B shows relative Rd1 adapter addition efficiencies for template switching (TSO), single-stranded splinted ligation (LIG) and both methods combined (TSO+LIG). FIG. 15A illustrates a workflow for the assay. Results are shown as Rd1 adapter addition efficiency fold change relative to TSO (FIG. 15B). Efficiency was calculated As 2^{-(Cq inner-Cq outer)} and then normalized relative to the TSO condition. Error bars are STDEV from 4 tissue sections.

FIG. 16 shows a schematic for library fragment size normalization with dUTP in reverse transcription reaction.

FIG. 17 shows the structure of the sequencing read.

FIG. 18 shows a schematic for library fragment size normalization with dUTP/allyl-T with in-tube second strand synthesis.

FIG. 19 shows library size normalization with ddNTPs in a reverse transcription reaction.

FIG. 20 demonstrates that ExAMP serves as a cDNA elution reagent and builds redundancy pre-sequencing.

FIG. 21 demonstrates that cDNA fragments can be shortened with 3′phos dNTPs or azido-ddNTPs.

FIG. 22 illustrates an example of how a SMI (e.g., a UMI) sequence is added during second strand cDNA synthesis in the TSO ligation methods.

### DETAILED DESCRIPTION

Isolating RNA from preserved tissue samples and converting RNA to cDNA on a flat surface presents a number of problems, including lower quality RNA transcripts isolated from the tissue samples, shorter synthesized cDNA fragments (<450 bp) in library preparation products and a high percentage of polyA presence in cDNA regions in the final sequencing products. These issues result in a subsequent low mapping rate to exonic mRNA transcript regions in RNA-seq alignment.

To solve this problem, it was hypothesized that an improved method to generate improved capture and spatial library conversion from FFPE tissue samples was needed.

Methods of the disclosure provide spatial RNA-sequencing library preparation methods, which can be used with fresh frozen tissue as well as formalin-fixed paraffin embedded tissue. Methods of the disclosure can utilize transposition-based methods to ensure spatial barcode remains intact during the library preparation method.

In one aspect, a template switch-tagmentation based method of the disclosure (TSO-TAG) utilizes a second strand priming and template switch oligonucleotide (TSO) process, with tagmentation to fragment and add UMI and adapters to the library fragments. An extension with an extension primer, such as Poly-TVN primer, pre-transposition can allow for the 3′ region of the fragment to remain single-stranded to avoid transposition on the spatial barcode. TSO-TAG methods of the disclosure can advantageously provide a simplified workflow for library preparation, with a reduction of second strand synthesis time to less than 2 hours, for example, 10 minutes to about 60 minutes or about 15 minutes to about 30 minutes. The resulting library fragments are full length since the TSO is added to the 5′ end of the transcript. The transposition adds the UMIs while fragmenting the library to the desired sequence and the barcode region remains single stranded during transposition.

In another aspect, a spatial barcode-tagmentation and UMI-tagmentation based method utilizes random second strand priming with global PCR to build redundancy. Tagmentation is used to fragment, while the randomer second strand priming adds the UMI and adapter.

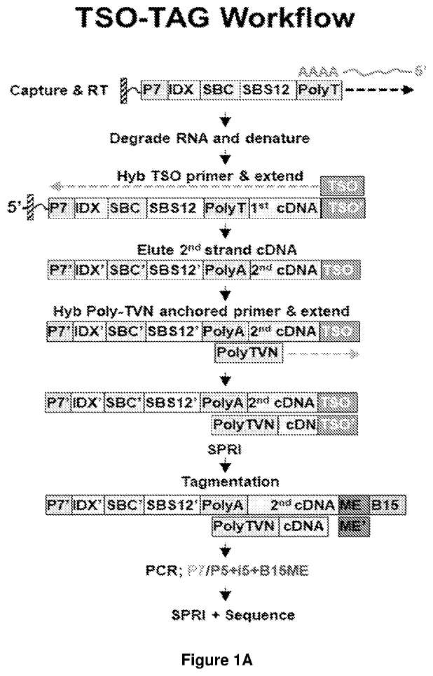

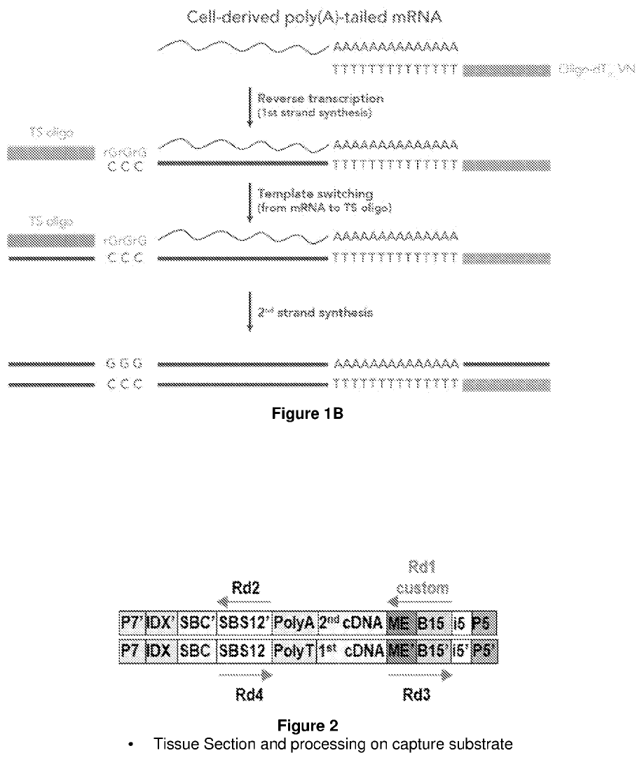

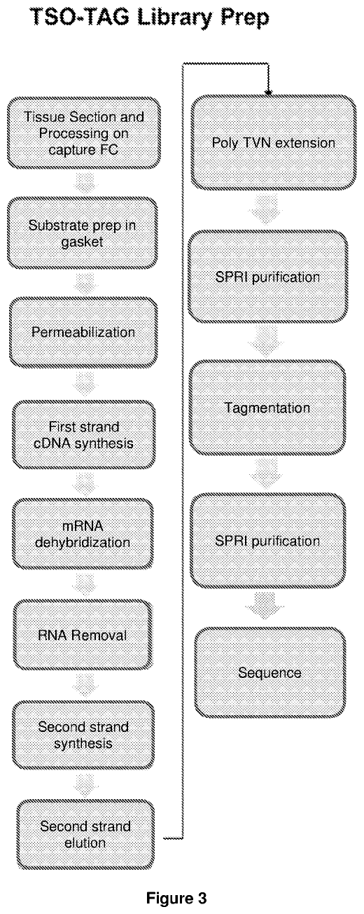

Referring to FIGS. 1 and 3, in accordance with an embodiment, a method for preparing an RNA sequence library can include mounting a tissue sample on a substrate comprising a plurality of capture oligonucleotides. The capture oligonucleotides can include one or more gene-specific capture sequences for capturing mRNA transcripts from the tissue. For example, the capture oligonucleotides can include a polyT sequence for capturing polyadenylated mRNA from the tissue. For example, fresh frozen tissue can be sectioned and fixed. The substrate can be, for example, part of a solid support. The solid support can be, for example, a flow cell. For example, an Illumina polyT barcoded, capture flow cell can be used. The flow cell can be assembled into a gasket that creates individual sample wells over the tissue sections, with the substrate defining the bottom surface of the wells. For methods using formalin fixed paraffin embedded tissue, the method can first include treating the tissue to polyadenylate formalin fixed paraffin embedded RNA transcripts to improve capture and library conversion when using polyT capture surface, for example. Other pretreatments of the tissue to improve capture with one or more gene specific sequences of the capture oligonucleotides can be used, as well.

Methods of the disclosure can include coating the substrate with an RNase inhibitor, such as 0.01×SSC/RNase Inhibitor, and the solution can be removed. The tissue can then be permeabilized by contacting the tissue and the substrate with a premeabilization mix and incubating the tissue in the mix. For example, the permeabilization mix can include 0.1% pepsin, 0.1N HCl and can be prewarmed. The tissue can be incubated, for example, for about 7 minutes at 37° C. The permeabilization mix can then be removed and the wells can be washed with buffer and RNase Inhibitor.

The capture oligonucleotides can include one or more gene-specific capture sequence or a polyT sequence and library barcode information comprising a spatial bar sequence (SBC). mRNA transcripts from the tissue are captured or immobilized on the substrate by the capture oligonucleotides.

Reverse transcriptase is performed with a template switch oligonucleotide, which adds the TSO complement to the 5′ end of the transcript. In particular, the substrate is contacted with a first strand synthesis mix that include a reverse transcriptase and a template switch oligonucleotide (TSO) under conditions to generate a first strand comprising a first cDNA that is complementary to the mRNA transcripts and a TSO complement hybridized to the 5′ end of the first cDNA. The reverse transcriptase incorporates untemplated cytosine nucleotides at the 5′ end of the first cDNA. The TSO includes a sequence that hybridizes to the untemplated cytosine nucleotides. The TSO hybridizes to the untemplated cytosine nucleotides and the reverse transcriptase is extended to generate the compliment of the TSO (referred to herein as the TSO complement) attached to the '5 end of the cDNA. For example, the untemplated cytosine nucleotides can be CCC. For example, the sequence that hybridizes to the untemplated cytosine nucleotides can be 2-5 guanosines. For example, the 2-5 guanosines can be riboguanosines. For example, the sequence that hybridizes to the untemplated cytosine nucleotides can be rGrGrG.

The first strand synthesis mix includes the reverse transcriptase and the TSO. The mix can further include a reducing agent, a reverse transcriptase reagent, and water. The components of the first strand synthesis mix can be premixed and added to the substrate or one or more of the components can be added step-wise to the substrate.

The substrate can be incubated in the first strand synthesis mix for any desired amount of time. For example, the incubation can be for about 1 hour at 53° C. The first strand synthesis mix is then discarded from the substrate and the substrate can be washed with water.

After the first strand is generated, the mRNA transcripts are eluted from the substrate. For example, elution can be performed using formamide. The substrate can be incubated in 100% formamide, for example, for 10 minutes at 80° C. The mRNA elution can be stored if desired at −80° C., for example, for use in reverse transcription qPCR quality control checks.

After elution, the substrate can be washed. For example, the substrate can be washed three times with water. KOH can be added to the substrate. The substrate can be incubated in the KOH at room temperature for 5 min, for example. The KOH solution is discarded and the substrate can be washed with a buffer, for example Buffer EB (Qiagen).

Second strand synthesis is then performed with a TSO primer. In particular, the substrate having the first strand is contacted with a second strand synthesis mix comprising a TSO primer and the TSO primer is extended using the first strand as a template to generate a second strand complimentary to the first strand and having a second cDNA complementary to the first cDNA. The second strand further includes second strand barcode information that has a spatial barcode sequence complement that is complementary to the barcode sequence present on the first strand (also referred to herein as the library barcode sequence). The second strand synthesis mix can include the TSO primer, a second strand reagent, and a second strand enzyme. The methods of the disclosure advantageously provide a process in which the time needed for second strand synthesis can be significantly reduced as compared to conventional processes. For example, the second strand synthesis can include incubating the first strand with the second strand synthesis mix for less than 2 hours. For example, the incubation time can be about 10 minutes to about 15 min, about 15 minutes to about 60 min, about 30 minutes to about 90 min, about 15 minutes to about 30, or about 45 minutes to about 2 hours. Other suitable times can be about 15, 16, 17, 18, 19, 20, 22, 24, 26, 28, 30, 32, 35, 36, 38, 40, 42, 44, 46, 48, 50, 52, 54, 56, 58, 60, 65, 70, 75, 80, 85, 90, 95, 100, 105, 110, 115, or 120 minutes and any ranges defined by such values, and any values there between. For example, the substrate can be incubated in the second strand synthesis mix at 65° C. for 15 minutes. The solution is discarded and the substrate can be washed with buffer, for example Buffer EB.

The second strand is then eluted from the substrate. For example, the elution can be performed by incubating the substrate in KOH at room temperature. For example, the substrate can be incubated for about 10 minutes in 0.08M KOH to elute the second strand. The eluted second strand in KOH can be transferred to a different reaction container. For example, strip tubes can be used. The eluted second strand in KOH can be neutralized before further extension. For example, Tris buffer can be used to neutralize the eluted second strand.

Extension with an extension primer is then performed to generate a double-stranded library while maintaining a single-stranded 3′ region containing the barcode information. For processes utilizing a polyT capture oligonucleotide, poly-TVN primer can be used as the extension primer. For example, the second strand is contacted with a poly-TVN extension mix comprising a Poly-TVN primer and extension is performed to generate a double-stranded product while maintaining a single stranded 3′ region containing the barcode information. For processes using a gene-specific capture oligonucleotide, the extension primer can be a primer that hybridizes to the second strand at a region of the second strand that does not include the barcode information. Extension can be achieved by admixing the second strand with extension primer mix and thermocycling. The extension primer mix can be, for example, Illumina AMS strand displacing extension mix can be used. Thermocycling can be performed, for example, with a second comprising a first temperature and a first time, a second temperature and a second time, and a hold temperature. The second temperature can be higher than the first temperature. The first temperature can be, for example, about 25° C. to about 37° C. The first time can be about 10 minutes to about 30 min. The second temperature can be about 60° C. to 65° C. The second time can be about 10 min. For example, thermocycling conditions can be 37° C. for 10 min, 60° C. for 10 min, and hold at 4° C.

The double stranded product can be purified using a known purification method. For example, SPRI purification can be used and the double stranded product can be eluted in water.

The double stranded product is contacted with a transposome under conditions to tagment the double stranded product to form a tagmented product that has a unique molecular identified and a PCR adapter. For example, Illumina's Surcecell B15 Tn5 transposome can be used for transposing. For example, the transposome can be an A14 or B15 transposome. For example, the transposome can have a custom transposon. The transposome can be provided as a transposome modified bead. The tagmentation process can include contacting the double stranded product with the transposome and a carrier gDNA. The concentration of carrier gDNA can be about 1 ng to about 10 nm. Tagmentation can be performed using diluted transposome and a tagmentation buffer. For example, a 5- to 20-fold dilution of the transposome can be used. Specific dilutions can be readily determined given the amount of product captured from the tissue. The double stranded product can be incubated in the transposome, for example, at 55° C. for 5 minutes. Tagmentation can be stopped using a tagment stop buffer. The double stranded product can be incubated with the tagment stop buffer, for example, for 5 minutes at room temperature.

Transposition can be performed as in-solution tagmentation, for example, Tn5 tagmentation. Alternatively, transposition can be performed on beads. For example, A14 transposome beads can be generated and used to transpose the double stranded product.

The tagmented product is amplified using index PCR to generate the library. Index PCR can be performed using a tagmentation PCR mix, P7 primer, and transposome-index-P5 primer. For example, the tagmented product can be amplified with P7 and a primer containing B15-ME, sample index, and P5. For transposition performed with A14 transposome, the amplification can be performed with P7/PA14 short. The index PCR process can include from 10 to 24 cycles. Each cycle can include for example, holding at a first temperature for a first time, holding for a second temperature for a second time, and holding for a third temperature for a third time, wherein the first temperature is higher than the second and the third temperatures, and the third temperature is higher than the second temperature. Each cycle can be about 15 to 50 minutes. For example, the cycle can include holding at 95° C. for 10 seconds, 60° C. for 45 seconds and a 72° C. for 60 second. The cycles can be preceded by a hold at the first temperature. After completion of all of the cycles, a final extension can be performed by holding at the third temperature. For example, the final extension can be held for about 5 to 10 minutes. For example, the initial hold pre-cycle can be at 95° C. for 30 seconds and the final extension at 72° C. for 5 min and a hold at 4° C.

The resulting library can be purified and sequenced. For example, the library can be purified with 1×SPRI.

FIG. 2 shows the sequencing read structure for the TAG-TSO. Read 1 reads into cDNA region. Read 2 reads into the spatial barcode. Read 3 reads into the sample index. Refer 4 reads into the Poly-TVN and cDNA region. In embodiments of the process not utilizing poly-TVN, the extension primer sequence would be in the position of the Poly-TVN.

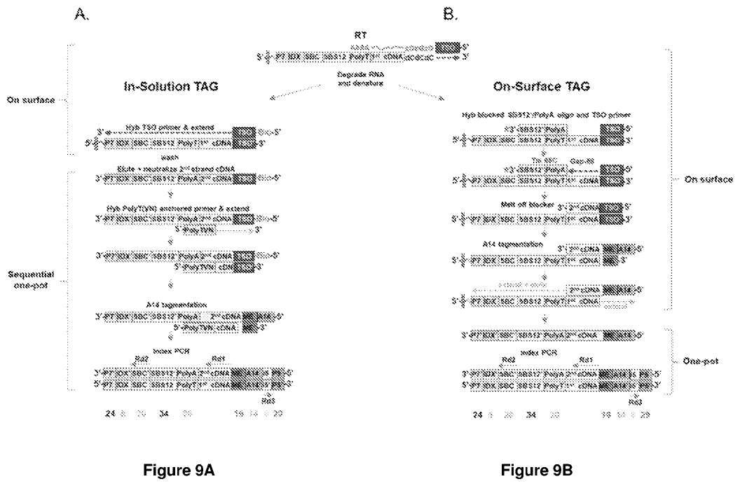

FIGS. 9A and 9B are schematic illustrations of TSO-TAG methods in accordance with the disclosure. In FIG. 9A, a method in which the process is performed in a sequential one-pot processes is illustrated. One-pot synthesis can be achieved by using a biotinylated TSO primer in the second strand synthesis step to generate a second strand having the biotinylated TSO. After elution and neutralization of the second strand cDNA, the product can be hybridized to beads and subsequent steps of the method can be performed on-bead with wash steps on magnet. For example, streptavidin beads can be used.

FIG. 9B illustrates an on-surface process for performing the TAG-TSO method of the disclosure. After RNA removal, a blocker oligonucleotide and TSO is hybridized to the first strand such that a gap is present between the blocker oligonucleotide and the TSO. The block oligonucleotide can be, for example, a 3′ blocked SBS12′-PolyA. A TSO compliment oligonucleotide then gap fills to 5′ region of the blocker oligonucleotide using non-strand displacing polymerase.

The blocker is then melted off to form a blocker-free first strand. On-surface tagmentation is performed, for example with an A14 transposome, to introduce the UMI and adapter. The tagmented product having the UMI and PCR adapter is then extended. Extension mix is added to form a second strand. The second strand includes second strand barcode information having a spatial barcode sequence complement (SBC′) complementary to the spatial barcode sequence and a second cDNA complementary to the first cDNA, the UMI, and the PCR adapter. The second strand product is eluted from the surface and amplified using index PCR;

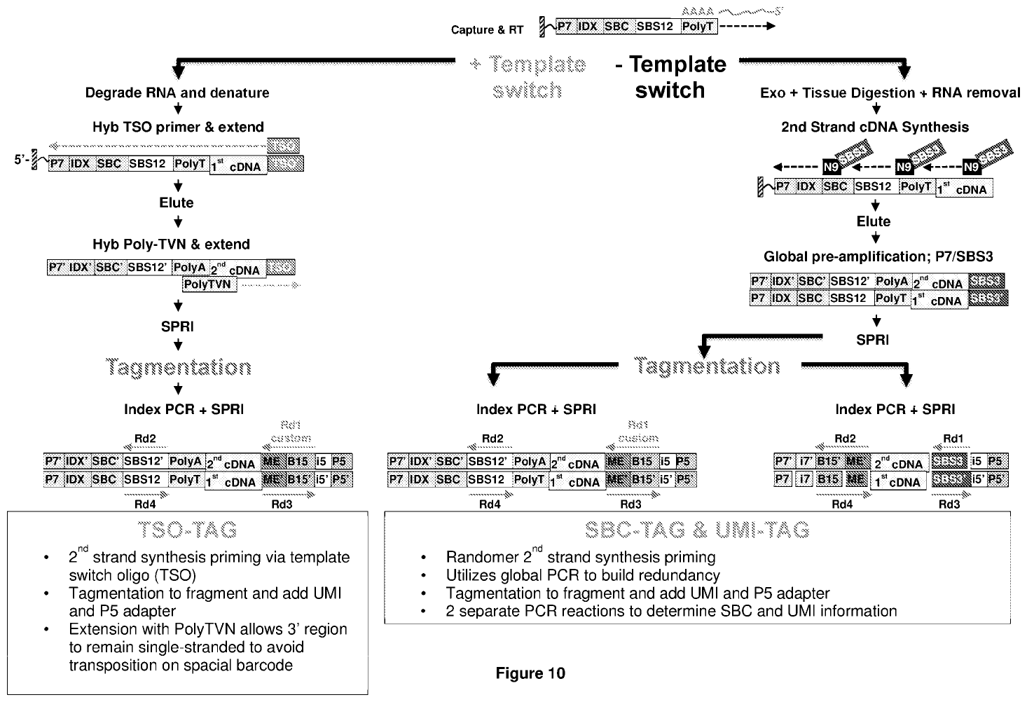

Referring to FIG. 10, the SBC-TAG UMI-TAG method is illustrated in comparison to the TSO-TAG method. The SBC-TAG, UMI-TAG method for preparing a RNA sequence library can include mounting a tissue sample on a substrate having a plurality of capture oligonucleotides. The substrate can be a flow cell or be part of a flow cell, for example. The capture oligonucleotides include a polyT sequence or one or more gene-specific capture sequences and barcode information comprising a spatial barcode sequence (SBC). mRNA transcripts from the tissue are captured on the substrate by the capture oligonucleotide. For capture oligonucleotides having the polyT sequence, polyadenylated mRNA is captured.

Reverse transcription using a first strand synthesis mix having a reverse transcriptase is performed to generate a first strand having a first cDNA complementary to the mRNA transcripts.

After the first strand is generated, the mRNA transcripts are eluted from the substrate. For example, elution can be performed using formamide. The substrate can be incubated in 100% formamide, for example, for 10 minutes at 80° C. The mRNA elution can be stored if desired at −80° C., for example, for use in reverse transcription qPCR quality control checks.

The second strand synthesis is performed using a random primer to generate a second strand having a second cDNA, UMI, and adapter. For example, the first strand is contacted with a second strand synthesis mix having the random primer and incubated to generate the second strand.

Alternatively, the second strand synthesis can be performed without elution of the mRNA. For example, after the first strand is generated and without elution of the mRNA transcripts, the second strand can be generated using a second strand synthesis mix that includes DNA pol 1 and RNase H. For example, Illumina second strand master mix can be used to generate the second strand. The first strand can be incubated with the second strand master mix, for example, at 16° C. for 1 hour to generate the second strand.

The second strand is then eluted.

The second strand product is then amplified to produce a double stranded product and the double stranded product is tagmented to form a tagmented product.

The tagmented products is amplified with a first index PCR to determine the SBC and amplified with a second index PCR to determine the UMI.

### Terms

As used in this specification and the enumerated paragraphs herein, the singular forms “a,” “an,” and “the” include plural reference unless the context clearly dictates otherwise.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

In the methods capture oligonucleotides are immobilized on a substrate via one or more polynucleotides, such as a polynucleotide. When referring to immobilization of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing. Oligonucleotides to be used as capture primers or amplification primers can be immobilized such that a 3′-end is available for enzymatic extension and at least a portion of the sequence is capable of hybridizing to a complementary sequence.

Immobilization can occur via hybridization to a surface attached oligonucleotide, in which case the immobilized oligonucleotide or polynucleotide can be in the 3′-5′ orientation. Alternatively, immobilization can occur by means other than base-pairing hybridization, such as the covalent attachment set forth above.

As used herein, the term “immobilized” refers to the state of two things being joined, fastened, adhered, attached, connected, or bound to each other. For example, an analyte, such as a nucleic acid, can be immobilized on a material, such as a bead, gel, or surface, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions. In various embodiments, covalent attachment can be used, but all that is required is that the oligonucleotides remain stationary or attached to a surface under conditions in which it is intended to use the surface, for example, in applications requiring nucleic acid capture, amplification, and/or sequencing.

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

The terms “solid surface,” “solid support” and other grammatical equivalents herein refer to any material that is appropriate for or can be modified to be appropriate for the attachment of the capture oligonucleotides. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flowcell apparatus. Additional non-limiting examples of solid supports and solid surfaces include a bead array, a spotted array, clustered particles arranged on a surface of a chip, and a multi-well plate.

As used herein, the term “substrate” is intended to mean a solid support or support structure. The term includes any material that can serve as a solid or semi-solid foundation for creation of features such as wells for the deposition of biopolymers, including nucleic acids, polypeptide and/or other polymers, including attachment of capture oligonucleotides. Non-limiting examples of substrates include a bead array, a spotted array, clustered particles arranged on a surface of a chip, a film, a multi-well plate, beads, and a flow cell. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

As used herein, “surface” can refer to a part of a substrate or support structure that is accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fibre; metal; inorganic glass; optical fibre bundle, or a variety of other polymers. A single material or mixture of several different materials can form a surface useful in certain examples. In some examples, a surface comprises wells (e.g., microwells or nanowells). In some aspects, the surface comprises an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some embodiments, each nanowell comprises a unique oligonucleotide (e.g., an oligonucleotide with a unique spatial barcode). In some examples, a support structure can include one or more layers. Non-limiting examples of a surface include a bead array, a spotted array, clustered particles arranged on a surface of a chip, a film, a multi-well plate, and a flow cell. In various embodiments, the substrate or surface of the substrate comprises a material selected from glass, silicon, poly-L-lysine coated materials, nitro-cellulose, polystyrene, cyclic olefin copolymers (COCs), cyclic olefin polymers (COPs), polyacrylamide, polypropylene, polyethylene, or polycarbonate.

Exemplary flow cells include but are not limited to those used in a nucleic acid sequencing apparatus such as flow cells for the Genome Analyzer®, MiSeq®, NextSeq® or HiSeq@ platforms commercialized by Illumina, Inc. (San Diego, Calif.); or for the SOLID™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference.

Those skilled in the art will know or understand that the composition and geometry of a substrate as provided herein can vary depending on the intended use and preferences of the user. Therefore, although planar substrates such as slides, chips wafers or beads are useful for microarrays, those skilled in the art will understand that a wide variety of other substrates exemplified herein or well known in the art also can be used in the methods and/or compositions herein.

In some embodiments, the solid support comprises one or more surfaces that are accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a surface include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fiber; metal; inorganic glass; optical fiber bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some examples, a support structure can include one or more layers.

Certain embodiments may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

In some embodiments, the solid support comprises a patterned surface suitable for immobilization of capture oligonucleotides in an ordered pattern. A “patterned surface” refers to an arrangement of different regions in or on an exposed layer of a solid support. For example, one or more of the regions can be features where one or more capture oligonucleotides are present. The features can be separated by interstitial regions where capture oligonucleotides are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the capture oligonucleotides are randomly distributed upon the solid support. In some embodiments, the captured oligonucleotides are distributed on a patterned surface. Exemplary patterned surfaces that can be used in the methods and compositions set forth herein are described in U.S. application Ser. No. 13/661,524 or US Pat. App. Publ. No. 2012/0316086 A1, each of which is incorporated herein by reference.

In some embodiments, the solid support comprises an array of wells (e.g., microwells or nanowells) or depressions in a surface. This may be fabricated as is generally known in the art using a variety of techniques, including, but not limited to, photolithography, stamping techniques, molding techniques and microetching techniques. In some aspects, the solid support comprises an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some examples, the solid support can include one or more layers. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flowcell.

In some embodiments, the solid support or its surface is non-planar, such as the inner or outer surface of a tube or vessel. In some embodiments, the solid support comprises microspheres or beads.

Attachment of a nucleic acid to a support, whether rigid or semi-rigid, can occur via covalent or non-covalent linkage(s). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid-phase.

In some embodiments, the solid support comprises microparticles, beads, a planar support, a patterned surface, or wells. In some embodiments, the planar support is an inner or outer surface of a tube.

The term “bead” refers to a small body made of a rigid or semi-rigid material. The body can have a shape characterized, for example, as a sphere, oval, microsphere, or other recognized particle shape whether having regular or irregular dimensions. Example materials that are useful for beads include, without limitation, glass; plastic such as acrylic, polystyrene or a copolymer of styrene and another material, polypropylene, polyethylene, polybutylene, polyurethane or polytetrafluoroethylene (TEFLON®, from Chemours); polysaccharides or cross-linked polysaccharides such as agarose or Sepharose; nylon; nitrocellulose; resin; silica or silica-based materials including silicon and modified silicon; carbon-fiber, metal; inorganic glass; optical fiber bundle, or a variety of other polymers. Example beads include, without limitation, controlled pore glass beads, paramagnetic beads, thoria sol, Sepharose beads, nanocrystals and others known in the art as described, for example, in Microsphere Detection Guide from Bangs Laboratories, Fishers Ind. Beads may also be coated with a polymer that has a functional group that can attach to an oligonucleotide. As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. A particularly useful material is glass. Other suitable substrate materials may include polymeric materials, plastics, silicon, quartz (fused silica), boro float glass, silica, silica-based materials, carbon, metals including gold, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g., being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in US Pat. App. Pub. No. 2012/0316086 A1 and 2013/0116153, the entire contents of each are incorporated by reference herein. In some embodiments, the solid support is a flow cell.

The beads need not be spherical; irregular particles may be used. Alternatively or additionally, the beads may be porous. The bead sizes range from nanometers, i.e., 100 nm, to millimeters, i.e., 1 mm, with beads from 0.2 micron to 200 microns, or from 0.5 to 5 microns, although in some embodiments smaller or larger beads may be used.

As used herein, the term “flow cell” is intended to mean a vessel having a chamber where a reaction can be carried out, an inlet for delivering reagents to the chamber and an outlet for removing reagents from the chamber. In some embodiments, the flow cells is a chamber comprising a solid surface across which one or more fluid reagents can be flowed. In some embodiments, the solid support comprises one or more surfaces of a flowcell. The term “flowcell” as used herein includes a chamber comprising a solid surface across which one or more fluid reagents can be flowed. The flow cell can be an ordered or random flow cell.

Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference. In some embodiments the chamber is configured for detection of the reaction that occurs in the chamber. For example, the chamber can include one or more transparent surfaces allowing optical detection of biological specimens, optically labeled molecules, or the like in the chamber. Exemplary flow cells include, but are not limited to those used in a nucleic acid sequencing apparatus such as flow cells for the Genome Analyzer®, MiSeq®, NextSeq® or HiSeq® platforms commercialized by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. The term can be similarly applied to proteins which are distinguishable as different from each other based on amino acid sequence differences.

By “complementary” is meant that an oligonucleotide comprises a sequence of nucleotides that can form a double-stranded structure by matching base-pairs with another oligonucleotide or part thereof. By “substantially complementary” is meant that the oligonucleotide has at least 85%, 90%, 95%, 98%, 99% or 100% overall sequence identity to the complementary sequence. In various embodiments, “complementary” oligonucleotides are 100% complementary to each other, while in other embodiments, a first oligonucleotide sequence is at least (meaning greater than or equal to) about 95% complementary to a second oligonucleotide sequence over the length of the first oligonucleotide, at least about 90%, at least about 85%, at least about 80%, at least about 75%, at least about 70%, at least about 65%, at least about 60%, at least about 55%, or at least about 50% complementary to the second oligonucleotide over the length of the first oligonucleotide to the extent that the oligonucleotides are able to hybridize to each other under the conditions being utilized. The percent complementarity is determined over the length of the oligonucleotide. For example, given a first oligonucleotide in which 18 of 20 nucleotides of the first oligonucleotide are complementary to a 20-nucleotide region in a second oligonucleotide of 100 nucleotides total length, the oligonucleotides would be 90 percent complementary. In this example, the remaining noncomplementary nucleotides may be clustered or interspersed with complementary nucleobases and need not be contiguous to each other or to complementary nucleotides.

As used herein, a “primer” is a nucleic acid molecule that can hybridize to a target sequence, such as an adapter attached to a library fragment. As one example, an amplification primer can serve as a starting point for template amplification and cluster generation. As another example, a synthesized nucleic acid (template) strand may include a site to which a primer (e.g., a sequencing primer) can hybridize in order to prime synthesis of a new strand that is complementary to the synthesized nucleic acid strand. Any primer can include any combination of nucleotides or analogs thereof. In some examples, the primer is a single-stranded oligonucleotide or polynucleotide. The primer length can be any number of bases long and can include a variety of non-natural nucleotides. In various embodiments, the sequencing primer is a short strand, ranging from 5 to 60 bases, from 10 to 60 bases, from 10 to 20 bases, from 10 to 30 bases, from 10 to 40 bases, from 10 to 50 bases, or from 20 to 40 bases.

As used herein, the term “molecular identifier,” “single molecule identifier,” or “SMI” refers to sequences of nucleotides applied to or identified in nucleic acid molecules that may be used to distinguish individual or groups of nucleic acid molecules from one another. When incorporated into a nucleic acid, a SMI can be used to correct for subsequent amplification bias by directly counting single molecular identifiers (SMIs) that are sequenced after amplification. A SMI (e.g., a UMI) can be attached to similar nucleic acids, e.g., adapters, making each nucleic acid unique. SMIs (e.g., UMIs) may also be used to uniquely tag individual molecules (e.g., individual mRNA molecules) in a sample (e.g., individual mRNA molecules in a tissue sample, cell sample, or sample library). In some embodiments, a UMI is a random nucleotide sequence (e.g., N9).

As used herein, the term “unique molecular identifier” or “UMI” refers to sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. A unique molecular index, unique molecular identifier or UMI, when used in reference to a capture probe or other nucleic acid is intended to refer to a portion of a probe useful as a molecular barcode to uniquely tag each molecule in a sample library. A UMI may be denoted as “NNNN . . . ” in a string of nucleic acids to designate that portion of the oligonucleotide as the UMI. A UMI may be from 6 to 20 nucleotides or more in length. UMIs are similar to barcodes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference. In some aspects, the UMI comprises a spatial barcode.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences. Universal capture oligonucleotides are applicable for interrogating a plurality of different oligonucleotides without necessarily distinguishing the different species whereas target-specific capture sequences are applicable for distinguishing the different species. A non-limiting example of a universal sequence is a polyT nucleotide sequence.

As used herein, a “semi-random” nucleotide sequence comprises or consists of a partially pre-determined nucleotide sequence combined with a random nucleotide sequence

As used herein, the terms “includes,” “including,” “includes,” “including,” “contains,” “containing,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

As used herein, the term “adapter” refers generally to any linear nucleic acid molecule that can be ligated to an oligonucleotide of the disclosure. In some embodiments, adapters include two reverse complementary oligonucleotides forming a double-stranded structure. In some embodiments, an adapter includes two oligonucleotides that are complementary at one portion and mismatched at another portion, forming a Y-shape or fork-shaped adapter that is double stranded at the complementary portion and has two floppy overhangs at the mismatched portion. In some embodiments, adapters are copied onto the library molecules using templated polymerase synthesis (e.g., second strand cDNA synthesis as described herein). In some embodiments, adapters are ligated to a first complementary strand of the disclosure. In some embodiments, an adapter comprises two oligonucleotides that are double-stranded at one portion and single-stranded at another portion, forming an adapter with an overhang. In some embodiments, an oligonucleotide primer comprises an adapter nucleotide sequence (e.g., a B15 nucleotide sequence). In some embodiments, an adapter comprises a sequence that is complementary to a primer. In further embodiments, an adapter comprises a sequence that is complementary to a P5 primer or a P5′ primer. In some embodiments, an adapter comprises a sequence complementary to a P7 primer or a P7′ primer. In some embodiments, an adapter comprises a sequence complementary to a B15 primer or a B15′ primer. The terms “P5”, “P7”, “B15”, “P5” (P5 prime), “P7” (P7 prime), “B15” (B15 prime), “P15”, “P17” and “A14” may be used when referring to examples of oligonucleotide sequences of primers, e.g., clustering primers, and/or oligonucleotide sequences that are complementary to primers. The terms “P5” (P5 prime), “P7” (P7 prime), “B15” (B15 prime) and “A14” (A14 prime) refer to the complement of P5, P7, B15 and A14, respectively. It will be understood that any suitable primer can be used in the methods presented herein, and that the use of P5, P5′, P7, P7′, P15, P17, B15, B15′, A14 and A14′ are exemplary embodiments only. Uses of primers such as P5, P5′, P7, P7′, P15, P17, B15, B15′, A14 and A14′ or their complements on flow cells are known in the art, as exemplified by the disclosures of WO 2019/222264, WO 2007/010251, WO 2006/064199, WO 2005/065814, WO 2015/106941, WO 1998/044151, and WO 2000/018957, each of which is incorporated herein by reference in its entirety.

For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein. In some embodiments, a “first clustering primer” as described herein is a P5 primer. In some embodiments, a “first clustering primer” as described herein is a P7 primer. In some embodiments, a “first clustering primer” as described herein is a P5′ primer. In some embodiments, a “first clustering primer” as described herein is a P7′ primer. In some embodiments, a “second clustering primer” as described herein is a P5 primer. In some embodiments, a “second clustering primer” as described herein is a P7 primer. In some embodiments, a “second clustering primer” as described herein is a P5′ primer. In some embodiments, a “second clustering primer” as described herein is a P7′ primer. In some embodiments, P5 comprises or consists of the polynucleotide sequence 5′ AAT GAT ACG GOG ACC ACC GA 3′ (SEQ ID NO: 1), or a variant thereof. In some embodiments, P5 comprises or consists of the polynucleotide sequence 5′ AAT GAT ACG GCG ACC ACC GAG ATC TAC AC 3′ (SEQ ID NO: 2), or a variant thereof. In some embodiments, P7 comprises or consists of the polynucleotide sequence 5′ CAA GCA GAA GAC GGC ATA CG 3′ (SEQ ID NO. 3), or a variant thereof. In some embodiments, P7 comprises or consists of the polynucleotide sequence 5′ CAA GCA GAA GAC GGC ATA CGA GAT 3′ (SEQ ID NO. 4), or a variant thereof. In some embodiments, P5′ comprises or consists of the polynucleotide sequence 5′ TCG GTG GTC GCC GTA TCA TT 3′ (SEQ ID NO: 5), or a variant thereof. In some embodiments, P5′ comprises or consists of the polynucleotide sequence 5′ GTG TAG ATC TCG GTG GTC GCC GTA TCA TT 3′ (SEQ ID NO: 6), or a variant thereof. In some embodiments, P7′ comprises the polynucleotide sequence 5′ CGT ATG CCG TCT TCT GCT TG 3′ (SEQ ID NO. 7), or a variant thereof. In some embodiments, P7′ comprises or consists of the polynucleotide sequence 5′ ATC TCG TAT GCC GTC TTC TGC TTG 3′ (SEQ ID NO. 8), or a variant thereof. In some embodiments, B15 comprises or consists of the polynucleotide sequence 5′ GTCTCGTGGGCTCGG 3′ (SEQ ID NO: 9), or a variant thereof. In some embodiments, B15′ comprises or consists of the polynucleotide sequence 5′ CCGAGCCCACGAGAC 3′ (SEQ ID NO: 10), or a variant thereof. In some embodiments, P15 comprises or consists of the polynucleotide sequence 5′ TTTTTTAATG ATACGGCGAC CACCGAGANC TACAC 3′ (SEQ ID NO: 11), or a variant thereof. In some embodiments, P17 comprises or consists of the polynucleotide sequence 5′ TTTTTTNNNC AAGCAGAAGA CGGCATACGA GAT 3′ (SEQ ID NO: 12), or a variant thereof. The term “variant” as used herein with reference to any of the sequences recited herein refers to a variant nucleic acid that is substantially identical, i.e., has only some nucleotide sequence variations, for example to the non-variant sequence. In some embodiments, a variant has at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99% overall nucleotide sequence identity to the non-variant nucleic acid sequence. It will be understood that reference to P5 and P7 herein could refer to different primer sequences. Any suitable primer sequence combinations are encompassed by the present disclosure.

As used herein an “anchor” refers to a moiety that attaches a nano-scaffold to a substrate. An anchor includes a chemical moiety, peptide, or oligonucleotide. A polynucleotide anchor may be between 4-20 nucleotides.

As used herein a “splint oligonucleotide” refers to an oligonucleotide comprising a sequence complementary to a region on a surface probe and another sequence complementary to a capture oligonucleotide, e.g., attached to a substrate. Splint oligonucleotides are typically 10 nucleotides or more in length. Splint oligonucleotides may be 10, 11, 12, 13, 14, 15, 16, 17, 18, 19, 20, 21, 22, 23, 24, 25, 30, 35, 40, 45, 50, 55, 60, 65, 60, 75, or 80 nucleotides.

As used herein a “surface oligonucleotide” refers to an oligonucleotide comprising an anchor sequence for attaching the oligo to the surface of a substrate, a spatial barcode sequence and a sequence that hybridizes with a splint oligonucleotide. Surface oligonucleotides are typically 20 nucleotides or more in length. Surface oligonucleotides may be 20, 21, 22, 23, 24, 25, 26, 27, 28, 29, 30, 35, 40, 45, 50, 55, 60, 65, 60, 75, or 80 nucleotides or more.

As used herein, the terms “address,” “tag,” “barcode” or “index,” when used in reference to a nucleotide sequence is intended to mean a unique nucleotide sequence that is distinguishable from other indices as well as from other nucleotide sequences within polynucleotides contained within a sample. A nucleotide “address,” “tag,” “barcode” or “index” can be a random or a specifically designed nucleotide sequence. An “address,” “tag,” “barcode” or “index” can be of any desired sequence length so long as it is of sufficient length to be unique nucleotide sequence within a plurality of indices in a population and/or within a plurality of polynucleotides that are being analyzed or interrogated. A nucleotide “address,” “tag,” “barcode” or “index” of the disclosure is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices.

As used herein, the term “barcode” is also intended to mean a series of nucleotides in an oligonucleotide that can be used provide barcode information including one or more of identification of the oligonucleotide, a spatial address on a surface, a characteristic of the oligonucleotide, or a manipulation that has been carried out on the oligonucleotide. The barcode can be a naturally occurring nucleotide sequence or a nucleotide sequence that does not occur naturally in the organism from which the barcoded nucleic acid was obtained. For example, each nucleic acid capture probe in a population on a substrate for spatial capture of nucleic acids in a biological sample, e.g., a permeabilized tissue sample, a cell suspension, can include different barcode sequences from all other nucleic acid capture probes in the population. Alternatively, each nucleic acid probe in a population can include different barcode sequences from some or most other nucleic acid capture probes in a population. For example, each capture probe in a population can have a barcode that is present for several different capture probes in the population even though the capture probes with the common barcode differ from each other at other sequence regions along their length. In various embodiments, one or more barcode sequences that are used with a biological tissue are not present in the genome, transcriptome or other nucleic acids of the biological specimen. For example, barcode sequences can have less than 80%, 70%, 60%, 50% or 40% sequence identity to the nucleic acid sequences in a particular biological tissue.

A tag/index/barcode sequence can be unique to a single nucleic acid species in a population or can be shared by several different nucleic acid species in a population. For example, each nucleic acid probe in a population can include different tag/index/barcode sequences from all other nucleic acid probes in the population. Alternatively, each nucleic acid probe in a population can include different tag/index/barcode sequences from some or most other nucleic acid probes in a population. For example, each probe in a population can have a tag/index/barcode that is present for several different probes in the population even though the probes with the common tag/index/barcode differ from each other at other sequence regions along their length. In particular embodiments, one or more tag/index/barcode sequences that are used with a biological specimen are not present in the genome, transcriptome or other nucleic acids of the biological specimen. For example, tag/index/barcode sequences can have less than 80%, 70%, 60%, 50% or 40% sequence identity to the nucleic acid sequences in a particular biological specimen.

As used herein, a “spatial address,” “spatial tag”, “spatial barcode”, “spatial barcode sequence” or “spatial index,” when used in reference to a nucleotide sequence, means an address, tag, barcode, or index encoding spatial information related to the region or location of origin of an addressed, tagged, barcoded, or indexed nucleic acid in a tissue sample. The sequence can be a naturally occurring sequence or a sequence that does not occur naturally in the organism from which the barcoded nucleic acid was obtained.

As used herein, a “template switch oligo” or “TSO” refers to an oligonucleotide useful in a method of DNA sequencing in which the oligonucleotide hybridizes to untemplated cytosine (C) nucleotides added to the end of a target RNA or DNA template by a reverse transcriptase during reverse transcription. For example, the TSO comprises a poly G sequence that binds the poly C sequence added to the target template. In some embodiments, the TSO comprises 2-5 guanosines that hybridizes to the untemplated cytosine nucleotides. In some embodiments, the 2-5 guanosines are riboguanosines, or modified or locked nucleic acids. In some embodiments, the TSO comprises rGrGrG.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatameric product of RCA). A first amplicon of a target nucleic acid can be a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

The number of template copies or amplicons that can be produced can be modulated by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. The number of copies of a nucleic acid template can be at least 1, 10, 100, 200, 500, 1000, 2000, 3000, 4000, 5000, 6000, 7000, 8000, 9000 and 10,000 copies, and can be varied depending on the particular application.

As used herein, the term “complementary” when used in reference to a polynucleotide is intended to mean a polynucleotide that includes a nucleotide sequence capable of selectively annealing to an identifying region of a target polynucleotide under certain conditions, e.g., a first oligonucleotide sequence can form a double-stranded structure by matching base-pairs with a second oligonucleotide sequence or portion thereof. In various embodiments, “complementary” oligonucleotides are 100% complementary to each other, while in other embodiments, a first oligonucleotide sequence is at least (meaning greater than or equal to) about 95% complementary to a second oligonucleotide sequence over the length of the first oligonucleotide, at least about 90%, at least about 85%, at least about 80%, at least about 75%, at least about 70%, at least about 65%, at least about 60%, at least about 55%, or at least about 50% complementary to the second oligonucleotide over the length of the first oligonucleotide to the extent that the oligonucleotides are able to hybridize to each other under the conditions being utilized. The percent complementarity is determined over the length of the oligonucleotide. For example, given a first oligonucleotide in which 18 of 20 nucleotides of the first oligonucleotide are complementary to a 20-nucleotide region in a second oligonucleotide of 100 nucleotides total length, the oligonucleotides would be 90 percent complementary. In this example, the remaining noncomplementary nucleotides may be clustered or interspersed with complementary nucleobases and need not be contiguous to each other or to complementary nucleotides. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single nucleic acid molecule having a particular sequence or a site can include several nucleic acid molecules having the same sequence (and/or complementary sequence, thereof). The sites of an array can be different features located on the same substrate. Exemplary features include without limitation, beads (or other particles) in or on a substrate, droplets, wells in a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C),thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl)ethylenediaminetriacetic acid that is capable of detection by delayed fluorescence, and the like.

As used herein, the terms “ligation,” “ligating,” and grammatical equivalents thereof are intended to mean to form a covalent bond or linkage between the termini of two or more nucleic acids, e.g., oligonucleotides and/or polynucleotides, typically in a template-driven reaction. The nature of the bond or linkage may vary widely, and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921, incorporated herein by reference in their entireties. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, the term “each,” when used in reference to a collection of items, is intended to identify an individual item in the collection but does not necessarily refer to every item in the collection unless the context clearly dictates otherwise.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g., DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. An extension reaction, in which nucleotides are added to the 3′ end of an oligonucleotide (e.g., a primer) is performed in the presence of a polymerase, such as a DNA or RNA polymerase. In some embodiments, the polymerase is a non-thermostable isothermal strand displacement polymerase. Suitable non-thermostable strand displacement polymerases according to the present disclosure can be found, for example, through New England BioLabs, Inc. and include phi29, Bsu, Klenow, DNA Polymerase I (E. coli), and Therminator. In some embodiments, the extension reaction is carried out by recombinase polymerase amplification (RPA). RPA comprises three core enzymes—a recombinase, a single-stranded DNA binding protein (SSB) and a strand-displacing polymerase. As described in Daher et al. (Rana K Daher, Gale Stewart, Maurice Boissinot, Michel G Bergeron, Recombinase Polymerase Amplification for Diagnostic Applications, Clinical Chemistry, Volume 62, Issue 7, 1 Jul. 2016). One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

Provided herein are arrays for and methods of spatial detection and analysis (e.g., mutational analysis or single nucleotide variation (SNV) detection as well as indel detection) of nucleic acid in a tissue sample. The arrays described herein can comprise a substrate on which a plurality of capture probes is immobilized such that each capture probe occupies a distinct position on the array. Some or all of the plurality of capture probes can comprise a unique positional tag (i.e., a spatial address or indexing sequence). A spatial address can describe the position of the capture probe on the array. The position of the capture probe on the array can be correlated with a position in the tissue sample.

As used herein, the term “poly T” or “poly A,” when used in reference to a nucleic acid sequence (e.g., a capture nucleotide sequence), is intended to mean a series of two or more thiamine (T) or adenine (A) bases, respectively. A poly T or poly A can include at least about 2, 5, 8, 10, 12, 15, 18, 20, 22, 25, 28, 30, 32, 35, 38, 40, or more of the T or A bases, respectively. Alternatively or additionally, a poly T or poly A can include at most about 40, 38, 35, 32, 30, 28, 25, 22, 20, 18, 15, 12, 10, 8, 5, or 2 of the T or A bases, respectively. In some embodiments, the disclosure contemplates use of a “polyTVN” sequence, wherein “T” is a capture nucleotide sequence, “V” is adenine (A), cytosine (C), or guanine (G), and “N” is adenine (A), cytosine (C), guanine (G), or thymine (T). The polyTVN sequence is used, in some embodiments, to bias reverse transcription to the base of the poly A tail on the mRNA molecule, e.g., in template switching.

As used herein, the term “tagmentation,” “tagment,” or “tagmenting” refers to transforming a nucleic acid, e.g., a DNA, into adaptor-modified templates in solution ready for cluster formation and sequencing by the use of transposase mediated fragmentation and tagging. This process often involves the modification of the nucleic acid by a transposome complex comprising transposase enzyme complexed with adaptors comprising transposon end sequence. Tagmentation results in the simultaneous fragmentation of the nucleic acid and ligation of the adaptors to the 5′ ends of both strands of duplex fragments. Following a purification step to remove the transposase enzyme, additional sequences are added to the ends of the adapted fragments by PCR.

A “transposase” refers to an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although many embodiments described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular embodiments, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998,J. Biol. Chem., 273:7367) or by a MuA transposase and a Mu transposon end comprising Rland R2 end sequences (Mizuuchi, 1983, Cell, 35:785; Savilahti et al., 1995, EMBO J., 14:4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183:2384-8; Kirby et al., 2002, Mol Microbiol, 43:173-86), Tyl (Devine and Boeke, 1994, NucleicAcids Res., 22:3765-72 and International Patent Application No. WO 95/23875), TransposonTn7 (Craig, 1996, Science. 271:1512; Craig, 1996, Review in: Curr Top MicrobiolImmunol, 204:27-48), TnIO and ISIO (Kleckner et al., 1996, Curr Top Microbiol Immunol, 204:49-82), Mariner transposase (Lampe et al., 1996, EMBO J., 15:5470-9), Tci (Plasterk, 1996, Curr Top Microbiol Immunol, 204:125-43), P Element (Gloor, 2004, Methods Mol Biol, 260:97-114), TnJ (Ichikawa and Ohtsubo, 1990, J Biol Chem. 265:18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86:2525-9), and retrotransposon of yeast (Boeke and Corces, 1989, Annu Rev Microbiol. 43:403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase. As used herein, the term “transposome complex” refers to a transposase enzyme non-covalently bound to a double stranded nucleic acid. For example, the complex can be a transposase enzyme pre-incubated with double-stranded transposon DNA under conditions that support non-covalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the gene of interest (e.g., nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, a “biological sample” may include one or more biological or chemical substances, such as nucleic acids, oligonucleotides, proteins, cells, tissues, organisms, and/or biologically active chemical compound(s), such as analogs or mimetics of the aforementioned species.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues. In some instances, the biological sample may include whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, viruses including viral pathogens, liquids containing multi-celled organisms, biological swabs and biological washes. In further examples, the sample can be derived from an organ, including for example, an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; an organ of the central nervous system such as brain, brainstem, cerebellum, spinal cord, cranial nerve, or spinal nerve; a sensory organ such as eye, ear, nose, or tongue; or an organ of the integument such as skin, subcutaneous tissue or mammary gland. In various embodiments, the tissue can be derived from a multicellular organism. In some embodiments, a tissue section can be contacted with a surface, for example, by laying the tissue on the surface. The tissue can be freshly excised from an organism, or it may have been previously preserved for example by freezing (e.g., fresh frozen tissue), embedding in a material such as paraffin (e.g., formalin fixed paraffin embedded (FFPE) samples), formalin fixation, infiltration, dehydration or the like. Optionally, a tissue section can be attached to a surface, for example, using techniques and compositions described in, for example, U.S. Pat. No. 11,390,912, incorporated by reference herein in its entirety. In some embodiments, a tissue can be permeabilized and the cells of the tissue lysed when the tissue is in contact with a surface. Any of a variety of treatments can be used such as those set forth above in regard to lysing cells. Target proteins and/or nucleic acids that are released from a tissue that is permeabilized can be captured by capture oligonucleotides on the surface. Thus, in various embodiments, the biological sample is a tissue sample. The thickness of a tissue sample or other biological sample that is contacted with a surface in a method set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a biological sample that is contacted with a surface will be no more than 100 μm, 50 μm, 10 μm, 5 μm, 1 μm, 0.5 μm, 0.25 μm, 0.1 μm or thinner.

As used herein, the term “tissue sample” refers to a piece of tissue that has been obtained from a subject, optionally fixed, sectioned, and mounted on a planar surface, e.g., a microscope slide. The tissue sample can be a formalin-fixed paraffin-embedded (FFPE) tissue sample or a fresh tissue sample or a frozen tissue sample, etc. The methods disclosed herein may be performed before or after staining the tissue sample. For example, following hematoxylin and eosin staining, a tissue sample may be spatially analyzed in accordance with the methods as provided herein. A method may include analyzing the histology of the sample (e.g., using hematoxylin and eosin staining) and then spatially analyzing the tissue. In various embodiments, the tissue is removed from the sample by enzymatic degradation. In various embodiments, the tissue removal is carried out before the RNA is removed from the tissue. In various embodiments, the tissue is removed via degradation with proteinase K, e.g., at 37° C. for 40 minutes.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the term “subject” encompasses mammals and non-mammals. Examples of mammals include, but are not limited to, any member of the mammalian class: humans, non-human primates such as chimpanzees, and other apes and monkey species, cattle, horses, sheep, goats, swine, rabbits, dogs, cats, rodents, rats, mice, guinea pigs, and the like. Examples of non-mammals include, but are not limited to, birds, fish, and the like. The term does not denote a particular age or gender.

The terms “P5” and “P7” may be used when referring to examples of adapters. The terms “P5” (P5 prime) and “P7” (P7 prime) refer to the complement of P5 and P7, respectively. It will be understood that any suitable adapter can be used in the methods presented herein, and that the use of P5 and P7 are exemplary embodiments only. Uses of adapters such as P5 and P7 or their complements on flowcells are known in the art, as exemplified by the disclosures of WO 2007/010251, WO 2006/064199, WO 2005/065814, WO 2015/106941, WO 1998/044151, and WO 2000/018957, each of which is incorporated herein by reference in its entirety. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, “hybridize” is intended to mean noncovalently associating a first oligonucleotide to a second oligonucleotide along the lengths of those polymers to form a double-stranded “duplex.” As used herein, the term “hybridization” refers to the process in which two single-stranded polynucleotides bind non-covalently to form a stable double-stranded polynucleotide. A resulting double-stranded polynucleotide is a “hybrid” or “duplex.” For instance, two DNA oligonucleotide strands may associate through complementary base pairing. The strength of the association between the first and second oligonucleotides increases with the complementarity between the sequences of nucleotides within those oligonucleotides. The strength of hybridization between oligonucleotides may be characterized by a temperature of melting (Tm) at which 50% of the duplexes have oligonucleotide strands that disassociate from one another. Oligonucleotides that are “partially” hybridized to one another means that they have sequences that are complementary to one another, but such sequences are hybridized with one another along only a portion of their lengths to form a partial duplex. Oligonucleotides with an “inability” to hybridize include those that are physically separated from one another such that an insufficient number of their bases may contact one another in a manner so as to hybridize with one another. Hybridization conditions will typically include salt concentrations of less than about 1 M, more usually less than about 500 mM and may be less than about 200 mM. A hybridization buffer includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances, and may be determined routinely by those skilled in the art.

As used herein, the term “plurality” is intended to mean a population of two or more members, which may all be the same or two or more members may be different. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “attached” refers to the state of two things being joined, fastened, adhered, connected or bound to each other. For example, an oligonucleotide can be attached to a material, such as a bead, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

In some embodiments, nucleic acids in a tissue sample are transferred to and captured onto an array. For example, a tissue section is placed in contact with an array and nucleic acid is captured onto the array and tagged with a spatial address. The spatially-tagged DNA molecules are released from the array and analyzed, for example, by high throughput next generation sequencing (NGS), such as sequencing-by-synthesis (SBS). In some embodiments, a nucleic acid in a tissue section (e.g., a formalin-fixed paraffin-embedded (FFPE) tissue section) is transferred to an array and captured onto the array by hybridization to a capture probe or capture oligonucleotide. In some embodiments, a capture oligonucleotide can be a universal capture probe hybridizing, e.g., to an adaptor region in a nucleic acid sequencing library, and/or to the poly-A tail of an mRNA. In some embodiments, the capture probe can be a gene-specific capture probe hybridizing, e.g., to a specifically targeted mRNA or cDNA in a sample, such as a TruSeq™ Custom Amplicon (TSCA) oligonucleotide probe (Illumina, Inc.). A capture oligonucleotide can be a plurality of capture oligonucleotides, e.g., a plurality of the same or of different capture oligonucleotides.

In some embodiments, a combinatorial indexing (addressing) system is used to provide spatial information for analysis of nucleic acids in a tissue sample. The combinatorial indexing system can involve the use of two or more spatial address sequences (e.g., two, three, four, five or more spatial address sequences).

In some embodiments, two spatial address sequences are incorporated into a nucleic acid during preparation of a sequencing library. A first spatial address can be used to define a certain position (i.e., capture site) in the X dimension on a capture array and a second spatial address sequence can be used define a position (i.e., a capture site) in the Y dimension on the capture array. During library sequencing, both X and Y spatial address sequences can be determined and the sequence information can be analyzed to define the specific position on the capture array.

In some embodiments, three spatial address sequences are incorporated into a nucleic acid during preparation of a sequencing library. A first spatial address can be used to define a certain position (i.e., capture site) in the X dimension on a capture array, a second spatial address sequence can be used to define a position (i.e., a capture site) in the Y dimension on the capture array, and a third spatial address sequence can be used to define a position of a two-dimensional sample section (e.g., the position of a slice of a tissue sample) in a sample (e.g., a tissue biopsy) to provide positional spatial information in the third dimension (Z dimension) of a sample. During library sequencing, X, Y, and Z spatial address sequences can be determined and the sequence information can be analyzed to define the specific position on the capture array.

In some embodiments, a temporal address sequence (T) is optionally incorporated into a nucleic acid during preparation of a sequencing library. In some embodiments, the temporal address sequence can be combined with two or three spatial address sequences. The temporal address sequence can, for example, be used in the context of a time-course experiment for determining time-dependent changes in gene-expression in a tissue sample. Time-dependent changes in gene-expression can occur in a tissue sample, for example, in response to a chemical, biological or physical stimulus (e.g., a toxin, a drug, or heat). Nucleic acid samples obtained at different timepoints from comparable tissue samples (e.g., proximal slices of a tissue sample) can be pooled and sequenced in bulk. An optional first spatial address can be used to define a certain position (i.e., capture site) in the X dimension on a capture array, a second optional spatial address sequence can be used to define a position (i.e., a capture site) in the Y dimension on the capture array, and a third optional spatial address sequence can be used to define a position of a two-dimensional sample section (e.g., the position of a slice of a tissue sample) in a sample (e.g., a tissue biopsy) to provide positional spatial information in the third dimension (Z dimension) of the sample. During library sequencing, T, X, Y, and Z address sequences are determined and the sequence information is analyzed to define the specific X, Y (and optionally Z) position on the capture array for each timepoint (T).

The address sequences X, Y, and, optionally, Z and/or T, can be consecutive nucleic acid sequences or the address sequences can be separated by one or more nucleic acids (e.g., 2 or more, 3 or more, 10 or more, 30 or more, 100 or more, 300 or more, or 1,000 or more). In some embodiments, the X, Y, and optionally Z and/or T address sequences can each individually and independently be combinatorial nucleic acid sequences.

In some embodiments, the length of the address sequences (e.g., X, Y, Z, or T) can each individually and independently be 100 nucleic acids or less, 90 nucleic acids or less, 80 nucleic acids or less, 70 nucleic acids or less, 60 nucleic acids or less, 50 nucleic acids or less, 40 nucleic acids or less, 30 nucleic acids or less, 20 nucleic acids or less, 15 nucleic acids or less, 10 nucleic acids or less, 8 nucleic acids or less, 6 nucleic acids or less, or 4 nucleic acids or less. The length of two or more address sequences in a nucleic acid can be the same or different. For example, if the length of address sequence X is 10 nucleic acids, the length of address sequence Y can be, e.g., 8 nucleic acids, 10 nucleic acids, or 12 nucleic acids.

Address sequences, e.g., spatial address sequences such as X or Y, can be either partially or fully degenerate sequences.

In some embodiments, spatially addressed capture probes on an array can be released from the array onto a tissue section for generation of a spatially addressed sequencing library. In some embodiments, a capture probe comprises a random primer sequence for in situ synthesis of spatially-tagged cDNA from RNA in the tissue section. In some embodiments, a capture probe is a TruSeq™ Custom Amplicon (TSCA) oligonucleotide probe (Illumina, Inc.) for capturing and spatially tagging genomic DNA in the tissue section. The spatially-tagged nucleic acid molecules (e.g., cDNA or genomic DNA) are recovered from the tissue section and processed in single tube reactions to generate a spatially-tagged amplicon library.

In some embodiments, magnetic nanoparticles can be used to capture nucleic acid (e.g., in situ synthesized cDNA) in a tissue sample for generation of a spatially addressed library.

In some embodiments, spatial detection and analysis of nucleic acid in a tissue sample can be performed on a droplet actuator.

Described herein are improved methods and compositions for spatial-omics applications that preserve spatial information related to the origin of RNA or DNA in the tissue. Examples of spatial omics applications include, but are not limited to, spatial genomic applications, spatial proteomic applications; spatial transcriptomic applications; spatial agrigenomic applications; spatial epigenomics s applications; spatial phenomic applications; spatial ligandomic applications; and spatial multiomic applications (e.g., transcriptomic and genomic applications).

### Oligonucleotides

An oligonucleotide is a polymer comprised of nucleotides. Oligonucleotides of the disclosure may be of any length and include, in various embodiments, DNA oligonucleotides, RNA oligonucleotides, analogs thereof, or a combination thereof. In any aspects or embodiments described herein, an oligonucleotide is single-stranded, double-stranded, or partially double-stranded.

Nucleotides may include naturally occurring nucleotides and functional analogs thereof. Examples of functional analogs are those that are capable of hybridizing to a nucleic acid in a sequence specific fashion or capable of being used as a template for replication of a particular nucleotide sequence. Naturally occurring nucleotides generally have a backbone containing phosphodiester bonds. An analog structure can have an alternate backbone linkage including any of a variety known in the art. Naturally occurring nucleotides generally have a deoxyribose sugar (e.g., found in DNA) or a ribose sugar (e.g., found in RNA). An analog structure can have an alternate sugar moiety including any of a variety known in the art. Nucleotides can include native or non-native bases. A native DNA can include one or more of adenine, thymine, cytosine and/or guanine, and a native RNA can include one or more of adenine, uracil, cytosine and/or guanine. Any non-native base may be used, such as a locked nucleic acid (LNA) and a bridged nucleic acid (BNA). Example modified nucleotides include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

Oligonucleotides contemplated by the disclosure also include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. Examples of universal bases include but are not limited to 5′-nitroindole-2′-deoxyriboside, 3-nitropyrrole, inosine and hypoxanthine.

In various aspects, an oligonucleotide of the disclosure, or a modified form thereof, is generally about 5 nucleotides to about 150 nucleotides in length. In further embodiments, an oligonucleotide of the disclosure is about 5 to about 125 nucleotides in length, about 5 to about 100 nucleotides in length, about 5 to about 90 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 150 nucleotides in length, about 10 to about 125 nucleotides in length, about 10 to about 100 nucleotides in length, about 10 to about 90 about 10 to about 50 nucleotides in length, about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more nucleotides in length. In various embodiments, the length of an oligonucleotide (such as a primer) of the disclosure is between about 5 base pairs (bp) and 40 bp, or between about 5 bp and 35 bp, or between about 5 bp and 30 bp, or between about 10 bp and 35 bp, or between about 10 bp and 30 bp, or between about 20 bp and 40 bp, or between about 20 bp and 35 bp, or between about 20 bp and 30 bp, or between about 9 and 20 bp or between about 5 and 15 bp, or between about 9 and 15 bp in length. In some embodiments, the length of an oligonucleotide (such as a primer) of the disclosure is about 10 bp, 13 bp, 15 bp, 20 bp, 25 bp, 30 bp, 35 bp, or 40 bp. As described herein, in various embodiments the oligonucleotide may be a P5 primer, a P5′ primer, a P7 primer, or a P7′ primer.

### Preparation of Polynucleotides

The present disclosure is based, in part, on the realization that the amount of RNA or DNA information isolatable from fresh or frozen tissue samples as well as FFPE tissue samples needs to be improved to provide information related to the genetic profile of the tissue sample. The present disclosure provides methods for improved capture of genetic information by increasing the amount and quality of RNA isolated from tissue samples that can be used in spatial transcriptomics analysis.

The total RNA can comprise ribosomal RNA (rRNA), messenger RNA (MRNA), transfer RNA (tRNA), microRNA, small nucleolar RNA (snoRNA), small nuclear RNA (snRNA). In various embodiments, the RNA is rRNA and/or mRNA.

In various embodiments, the RNA capture oligonucleotide is selected from the group consisting of a poly-T sequence, a randomer, a semi-randomer, or a target-specific probe. In various embodiments, the target-specific probes comprise a plurality of different target-specific RNA capture probe sequences. In various embodiments, the RNA capture probe or surface capture probe is between 8 to 80 nucleotides. In certain embodiments, the RNA capture probe or surface probe is between 10 to 80 nucleotides, between 10 to 70 nucleotides, between 10 to 60 nucleotides, between 10 to 50 nucleotides, between 10 to 40 nucleotides, between 10 to 30 nucleotides, between 10 to 20 nucleotides, between 20 to 80 nucleotides, between 20 to 70 nucleotides, between 20 to 60 nucleotides, between 20 to 50 nucleotides, between 20 to 40 nucleotides, or is 8, 9, 10, 11, 12, 13, 14, 15, 16, 17, 18, 19, 20, 21, 22, 23, 24, 25, 26, 27, 28, 29, 30, 40, 50, 60, 70 or 80 nucleotides.

A capture oligonucleotide as described herein can comprise a capture sequence, a spatial barcode sequence (SBC), and adapter sequences. Capture sequences include a poly-T sequence, a poly-A sequence, a gene-specific capture sequence, or a universal capture sequence. A universal capture sequence is a random nucleotide sequence or a non-self complementary semi-random sequence. In various embodiments, the capture oligonucleotide comprises a 5′ clustering sequence, a randomized spatial barcode (SBC), a full-length read 2 adapter sequence (Rd2 FL), a molecular identifier (MI), a fixed sequence (FS), and/or a poly T capture sequence with a 3′ VN terminus (polyTVN).

The oligonucleotides comprising a surface oligo nucleotide (e.g., poly T sequences) can further comprise spatial index sequences, including, but not limited to, one or more of a P7 sequence, an index sequence, and/or a read 2 adapter sequence (Rd2 FL). In various embodiments, the surface oligonucleotide comprises a P7 anchor sequence, a spatial barcode and a sequence that hybridizes with a splint oligonucleotide.

In some embodiments, the total RNA is released from the tissue sample. Release includes lysis of tissue or permeabilization of the tissue. In various embodiments, one or more samples that have been contacted with a solid support can be lysed to release target nucleic acids. Lysis can be carried out using known techniques, such as those that employ one or more of chemical treatment, enzymatic treatment, electroporation, heat, hypotonic treatment, sonication or the like. It is contemplated that the tissue sample is permeabilized prior to contacting the tissue sample with a plurality of capture oligonucleotides in the methods. In various embodiments, the tissue sample is treated with one or more blocking reagents prior to contacting the tissue sample with a plurality of capture oligonucleotides i the methods. In various embodiments, the tissue sample is permeabilized and treated with one or more blocking reagents prior to step contacting the tissue sample with a plurality of capture oligonucleotides in the methods.

In some embodiments, a tissue sample will be treated to remove embedding material (e.g., to remove paraffin or formalin) from the sample prior to release, capture or modification of nucleic acids. This can be achieved by contacting the sample with an appropriate solvent (e.g., xylene and ethanol washes). Treatment can occur prior to contacting the tissue sample with a solid support set forth herein or the treatment can occur while the tissue sample is on the solid support. It is also contemplated that the tissue is removed from the sample by enzymatic degradation. In various embodiments, the tissue removal is carried out before the RNA is removed from the tissue. In various embodiments, the tissue is removed via degradation with proteinase K, e.g., at 37° C. for 40 minutes. Exemplary methods for manipulating tissues for use with solid supports to which nucleic acids are attached are set forth in US Pat. App. Publ. No. 2014/0066318, which is incorporated herein by reference.

A formalin-fixed tissue sample may also be decrosslinked using known techniques. In various embodiments, decrosslinking is carried out using Tris-EDTA (TE) buffer, e.g., at pH 8, pH 9, or another appropriate buffer at an appropriate pH. Decrosslinking may also be carried out at high heat, e.g., 70° C.

The methods above are also useful for improving capture efficiency of RNA transcripts for in situ RNA transcript library preparation, and/or for improving the nucleotide length of polynucleotides used in generating an in situ transcriptome library (e.g., improving the polynucleotide size of cDNA transcribed from mRNA isolated from a sample and used in generating an in situ transcriptome library).

### Spatial Detection and Analysis of Nucleic Acids in a Tissue Sample

According to the methods described herein, spatial detection and analysis of nucleic acids in a tissue sample can be performed using sets of two or more capture probes (e.g., 3 or more, 4 or more, 5 or more, 6 or more, 7 or more, 8 or more, 9 or more, or 10 or more capture probes). Typically, at least a first capture probe in a set of capture probes is immobilized on a capture array or a nanostructure. In some embodiments, a second capture probe can be immobilized on the same capture array as the first capture probe, e.g., in proximity to the first capture probe, e.g., in the same capture site. In some embodiments, a second capture probe can be immobilized on a nanostructure or a particle, such as a magnetic particle or a magnetic nanoparticle. In some embodiments, a second capture probe can be in solution, e.g., to be used to perform in situ reactions with a nucleic acid in a tissue sample. The capture probes in the capture probe sets individually and independently can have a variety of different regions, e.g., a capture region (e.g., a first universal or gene-specific capture region or first clustering region), a primer binding region (e.g., a SBS primer region, such as a SBS3 or SBS12 region), or a second universal region/clustering sequence, such as a P5 or P7 region, a spatial address region (e.g., a partial or combinatorial spatial address region), or a cleavable region.

Exemplary sequences include the following Rd1 and Rd2 adaptor sequences. Second Universal Adapter-Rd1 SBS3 (long):

(SEQ ID NO: 13)
ACACTCTTTCCCTACACGACGCTCTTCCGATCT;

Second Universal Adapter-Rd1 SBS3 (short): ACACTCTTTCCCTACACGAC (SEQ ID NO: 14); First Universal Adapter-Rd2 SBS12 (long):

(SEQ ID NO: 15)
GTGACTGGAGTTCAGACGTGTGCTCTTCCGATCT;

First Universal Adapter—Rd2 SBS12 (short): GTGACTGGAGTTCAGACGTGT (SEQ ID NO: 16).

In some embodiments, only one capture probe in a set of capture probes comprises a capture region. In some embodiments, two or more capture probes in a set of capture probes comprise as capture region.

In some embodiments, only one probe in a set of capture probes comprises a spatial address region, e.g., such as a complete spatial address region describing the position of a capture site on a capture array. In some embodiments, two or more probes in a set of capture probes can comprise a spatial address region, e.g., two or more probes can each comprise a partial spatial address region (i.e., combinatorial address region), wherein each partial address region describes the position of a capture site on a capture array, e.g., along the x-axis or the y-axis.

In some embodiments, a set of capture probes (e.g., a RNA and surface capture probe) can comprise at least one capture probe comprising a capture region and a spatial address region (e.g., a complete or a partial spatial address region). In some embodiments, no capture probe in a set of capture probes comprises both a capture region and a spatial address region.

In some embodiments, the capture site on the substrate is a plurality of capture sites. In some embodiments, the plurality of capture sites is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or 1,000,000,000 or more capture sites.

In various embodiments, the capture array or substrate comprises a capture site density of 1 or more, 2 or more, 10 or more, 30 or more, 100 or more, 300 or more, 1,000 or more, 3,000 or more, 10,000 or more, 100,000 or more, 1,000,000 or more, capture sites per square centimeter (cm²).

In various embodiments, the pair of capture probes in a capture site is a plurality of pairs of capture probes. In some embodiments, the plurality of capture probes is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or more, 100,000,000 or more, or 1,000,000,000 or more capture probes.

In some embodiments, the pair of capture probes in a capture site of a substrate is a plurality of pairs of capture probes. In some embodiments, each RNA capture probe in the plurality of pairs of capture probes within the same capture site comprises the same spatial address sequence. In some embodiments, each RNA capture probe in the plurality of pairs of capture probes in different capture sites comprises a different spatial address sequence.

In some embodiments, the surface of the capture array is a planar surface, e.g., a glass surface. In some embodiments, the surface of the capture array comprises one or more wells. In some embodiments, the one or more wells correspond to one or more capture sites. In some embodiments, the surface of the capture array is a bead surface.

In some embodiments, the capture region in the surface capture probe is a gene-specific capture region. In some embodiments, the gene-specific capture region in the surface capture probe comprises the sequence of a TruSeq™ Custom Amplicon (TSCA) oligonucleotide probe (Illumina, Inc.). For example, the gene-specific capture regions in a plurality of second capture probes in a capture site can comprise a plurality of sequences of TSCA oligonucleotide probes.

In another embodiment, the disclosure provides for a substrate, such as a flowcell, nanoparticles or beads, which comprise the spatially addressable probes disclosed herein. In a particular embodiment, beads comprise the spatially addressable probes disclosed herein. In a further embodiment, the bead comprises streptavidin on the surface of the bead. In yet a further embodiment, the beads comprise a plurality of oligos bound to the bead via a linkage or a reversible linkage. Examples of reversible linkages include biotin molecule(s), such as ddBio molecules. The oligos bound the substrate typically comprise an adaptor sequence, such as P5 sequence or a P7 sequence. As used herein a P5 sequence comprises a sequence defined by AAT GAT ACG GCG ACC ACC GA (SEQ ID NO: 1) or AAT GAT ACG GCG ACC ACC GAG ATC TAC AC (SEQ ID NO: 2) and a P7 sequence comprises a sequence defined by CAA GCA GAA GAC GGC ATA CG (SEQ ID NO: 3) or CAA GCA GAA GAC GGC ATA CGA GAT (SEQ ID NO: 4). In some embodiments, the P5 or P7 sequence can further include a spacer polynucleotide, which may be from 1 to 20, such as 1 to 15, or 1 to 10, nucleotides, such as 2, 3, 4, 5, 6, 7, 8, 9, or 10 nucleotides in length. In some embodiments, the spacer includes 10 nucleotides. In some embodiments, the spacer includes 10 nucleotides. In some embodiments, the spacer is a polyT spacer, such as a 10T spacer. Spacer nucleotides may be included at the 5′ ends of polynucleotides, which may be attached to a suitable support via a linkage with the 5′ end of the oligo. Attachment can be achieved through a sulfur-containing nucleophile, such as phosphorothioate, present at the 5′ end of the polynucleotide. In some embodiments, the oligos will include a polyT spacer and a 5′phosphorothioate group. Thus, in some embodiments, the P5 sequence comprises 5′phosphorothioate-TTTTTTTTTTAATGATACGGCGACCACCGA-3′ (SEQ ID NO: 17), and in some embodiments, the P7 sequence comprises 5′ phosphorothioate-TTTTTTTTTTCAAGCAGAAGACGGCATACGA-3′ (SEQ ID NO: 18). In certain embodiments, the oligos attached to the beads comprise an address sequence that allows for determining the x, y position of the oligo/bead when decoded. In further embodiments, the address sequence is 5, 10, 15, 20, 25, 30, 35, 40, 45, 50, 55, 60, 65, 70, 75, 80, 85, 90, 95, or 100 nucleotides in length, or a range that includes or is between any two of the foregoing nucleotides in length. In another embodiment, the oligos attached to beads comprising a transposome hybridization region (Tsm hyb). In yet additional embodiments, the oligos comprise sequencing primer(s) site sequence(s). Examples of sequencing primer site sequences include sequences that are complementary to R1 and R2 sequencing primers from Illumina™. In further embodiments, the oligos may further comprise one or more linker sequences. In yet further embodiment, the oligos may further comprise one or more index sequences. In certain embodiments, the oligos may comprise one or more unique molecular identifier (UMI) sequences. Unique molecular identifiers (UMIs) are a type of molecular barcoding that provides error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. As noted previously, the oligos comprise moieties or sequences that can bind with specificity to polynucleotides from a biological sample (e.g., a tissue sample). As such, the oligos attached to the beads are spatially addressable probes for polynucleotides from a biological sample. The moieties or sequences that can bind with specificity to polynucleotides from a biological sample can be selected for a particular-omic application. For example, the oligos can comprise an oligo d (T) sequence for transcriptomics or for assay (e.g., RNA-seq assays). Alternatively, the oligos can comprise sequences to bind with genomic DNA from a biological sample for genomic applications or for assays (e.g., ATAC-seq assays). As provided in the Examples presented herein, the nanostructures can comprise multiple types of oligos that have different moieties or sequences so that the spatially addressable probes can bind specifically to two or more different types of polynucleotides from a biological sample. The use of multi types of oligos is ideally suited for multiomic or multiple assay applications.

Generation of second complementary strands may be performed “on surface” or “off surface”. In some embodiments, for on surface generation of second complementary strands the first complementary strands remain immobilized on the surface while second complementary strands are extended using the first complementary strands as template. In some embodiments, the second complementary strands are removed (eluted) from the surface, after which the second complementary strands are subjected to indexed PCR for amplification. In some embodiments, for on surface generation of second complementary strands an Exclusion Amplification (ExAmp) mix comprising an adapter-index oligonucleotide is contacted with the first complementary strands on the surface, thereby generating second complementary strands via strand invasion and isothermal amplification. In various embodiments, the ExAmp mix further comprises a recombinase, a single-strand DNA binding protein (e.g., gp32 ssDNA binding protein), and a polymerase. As with any of the methods of generating second complementary strands described herein, generation of the second complementary strands may subsequently be followed by amplification of the second complementary strands (e.g., by indexed PCR), during which a second clustering primer sequence (e.g., P5) may be added to one or more of the second complementary strands. In some embodiments, the amplifying comprises index PCR during which a first primer hybridizes to the first clustering primer sequence and a second primer hybridizes to the adapter nucleotide sequence, wherein the second primer comprises the second clustering primer sequence. In various embodiments, an indexing sequence (e.g., i5) is also added to one or more of the second complementary strands during amplification. In some embodiments, the second primer further comprises the indexing sequence. Addition of the second clustering primer sequence and optionally the indexing sequence to the one or more of the second complementary strands occurs, in various embodiments, off of the surface (e.g., in solution). The amplification of the second complementary strands may subsequently be followed by sequencing. The sequencing information may subsequently be correlated with a spatial location of the target nucleic acids in the biological sample.

In some embodiments, one or more of the plurality of capture oligonucleotides is immobilized on the surface through a cleavage site. In further embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In further embodiments, one or more of the plurality of capture oligonucleotides is immobilized on the surface through a cleavage site. In some embodiments, the cleavage site is an enzymatic cleavage site. In further embodiments, the enzymatic cleavage site comprises a restriction enzyme site, a uracil, an 8-oxoguanine, or a combination thereof.

In some embodiments, for off surface generation of second complementary strands formation of the first complementary strands is followed by cleavage of the first complementary strands from the surface, and second complementary strand synthesis is performed off of the surface (e.g., in solution). In some embodiments, the second complementary strands are then amplified (e.g., via indexed PCR), during which a second clustering primer sequence (e.g., P5) may be added to one or more of the second complementary strands. In various embodiments, an indexing sequence (e.g., i5) is also added to one or more of the second complementary strands during amplification. Addition of the second clustering primer sequence and optionally the indexing sequence to the one or more of the second complementary strands occurs, in various embodiments, off of the surface (e.g., in solution). The amplification of the second complementary strands may subsequently be followed by sequencing. The sequencing information may subsequently be correlated with a spatial location of the target nucleic acids in the biological sample.

Methods of the disclosure further provide, in various embodiments, that the biological sample/tissue sample is digested. The digestion of the biological sample can occur, in various embodiments, after generation of the first complementary strands. In some embodiments, digestion of the biological sample occurs after generation of the first complementary strands but prior to generation of second complementary strands. The disclosure also provides methods in which the target nucleic acids (e.g., RNA) are removed from the surface. Removal of target nucleic acids from the surface can occur, in various embodiments, after generation of the first complementary strands. In some embodiments, removal of the target nucleic acids occurs after generation of the first complementary strands but prior to generation of second complementary strands. Removal of the target nucleic acids from the surface is achieved, in various embodiments, by changing a condition. In further embodiments, the condition is temperature, pH, formamide concentration, or a combination thereof.

Aspects of the disclosure include those in which a plurality of capture oligonucleotides is immobilized on a surface. The capture oligonucleotides, in various embodiments, hybridize to target nucleic acids of a biological sample. In some embodiments, each of the plurality of capture oligonucleotides comprises the same capture nucleotide sequence. In further embodiments, the plurality of capture oligonucleotides comprises multiple, different capture nucleotide sequences. In still further embodiments, the multiple, different capture nucleotide sequences comprise one or more gene-specific capture sequences, one or more universal capture sequences, or a combination thereof. In various embodiments, the capture nucleotide sequence is a poly-T sequence, a poly-A sequence, a gene-specific capture sequence, or a universal capture sequence. In further embodiments, the universal capture sequence is a random nucleotide sequence or a non-self complementary semi-random sequence. Aspects of the disclosure also include those in which capture nucleotide sequences are extended following hybridization of the capture oligonucleotide to the target nucleic acid. In some embodiments, the extending of the capture nucleotide sequence is carried out using a reverse transcriptase. In some implementations of a method of the disclosure, the target nucleic acids are polyadenylated prior to hybridization of the target nucleic acids to the capture nucleotide sequences. In some embodiments, the target nucleic acids are polyadenylated using a poly(A) polymerase. In further embodiments, the target nucleic acids are polyadenylated using chemical ligation or enzymatic ligation.

### Methods of Normalizing Library Size

In one aspect, the disclosure is directed to methods of normalizing library size for on-surface library preparation applications. In various aspects the disclosure also provides methods for spatially capturing target nucleic acids of a tissue sample. Use of methods of the disclosure provides the ability to spatially preserve the location of target nucleic acids in a biological sample (e.g., tissue sample). The technology disclosed herein provides several advantages, including but not limited to: (1) the technology of the disclosure provides the ability to capture target analytes (e.g., mRNA or other analytes for multi-omic approaches) on a surface (e.g., an Illumina flow cell (Illumina Inc., San Diego Calif.)) followed by sequencing readout using an appropriate (e.g., Illumina Inc., San Diego Calif.) sequencing infrastructure. Such technology allows untargeted spatial detection of mRNA/analytes to allow for de-novo mapping of signals in the tissue context; (2) technology of the disclosure provides the ability to generate spatial transcriptomic libraries that are of a size that is optimal for sequencing. For example, and without limitation, in some embodiments, methods of the disclosure allow for generation of transcriptomic libraries comprising fragments of target analytes (e.g., target nucleic acids) that are about 100-1000 nucleotides in length. In further embodiments, methods of the disclosure allow for generation of transcriptomic libraries comprising fragments of target analytes (e.g., target nucleic acids) that are about 100-800 nucleotides in length. In some embodiments, methods of the disclosure allow for generation of transcriptomic libraries comprising fragments of target analytes (e.g., target nucleic acids) that are about 800 nucleotides in length. In some embodiments, methods of the disclosure allow for generation of transcriptomic libraries comprising fragments of target analytes (e.g., target nucleic acids) that are about 700 nucleotides in length.

In various aspects, the disclosure provides methods of preparing an immobilized library of target nucleic acids of a biological sample, comprising providing a surface comprising capture oligonucleotides that hybridize or otherwise associate with the target nucleic acids of the biological sample, extending (e.g., via reverse transcription) the capture oligonucleotides to form first complementary strands of the target nucleic acids, thereby preparing the immobilized library of target nucleic acids. In some embodiments, following formation of the first complementary strands a plurality of oligonucleotide primers is hybridized to the first complementary strands and the plurality of oligonucleotide primers is extended, thereby generating one or more second complementary strands. As described above, technology of the disclosure provides the ability to generate spatial transcriptomic libraries that are of a size that is optimal for sequencing. To normalize library fragment size, methods of the disclosure include use of an extension termination moiety during the extension of the capture oligonucleotides to form first complementary strands. The extension termination moieties act to terminate synthesis of a growing nucleic acid strand. Extension termination moieties contemplated by the disclosure include, but are not limited to, an allyl-T or a deoxyuridine triphosphate (dUTP), a dideoxynucleoside triphosphate (ddNTP), a deoxynucleoside triphosphate (dNTP) comprising a 3′ phosphate, a deoxynucleoside triphosphate (dNTP) comprising a 3′ phosphate, a dideoxynucleoside triphosphate (ddNTP) comprising a first click chemistry handle, or a combination thereof.

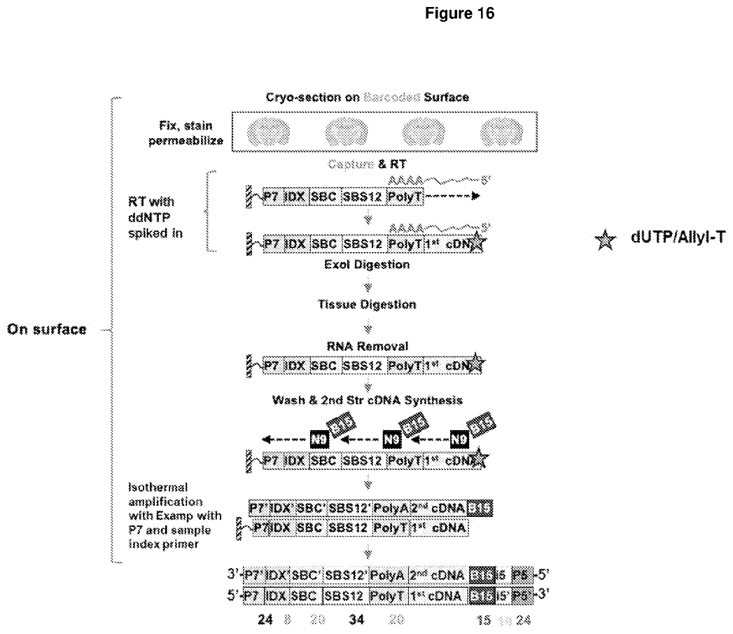

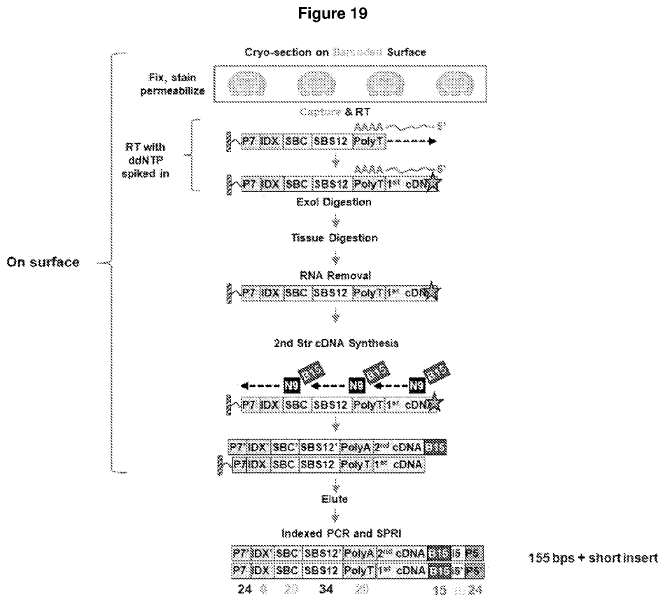

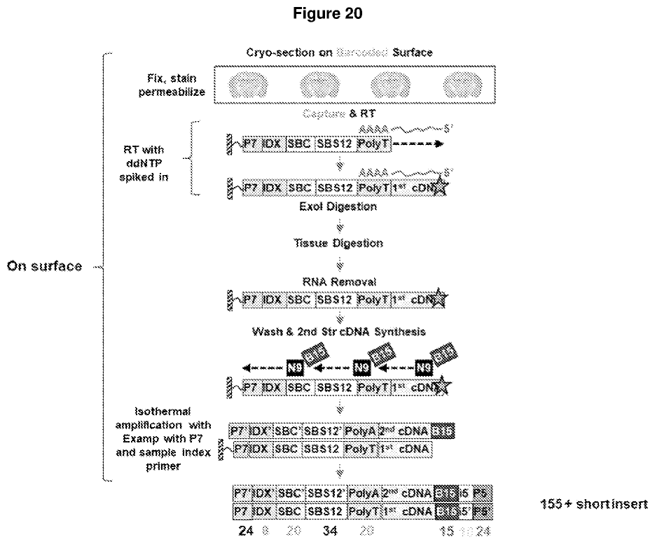

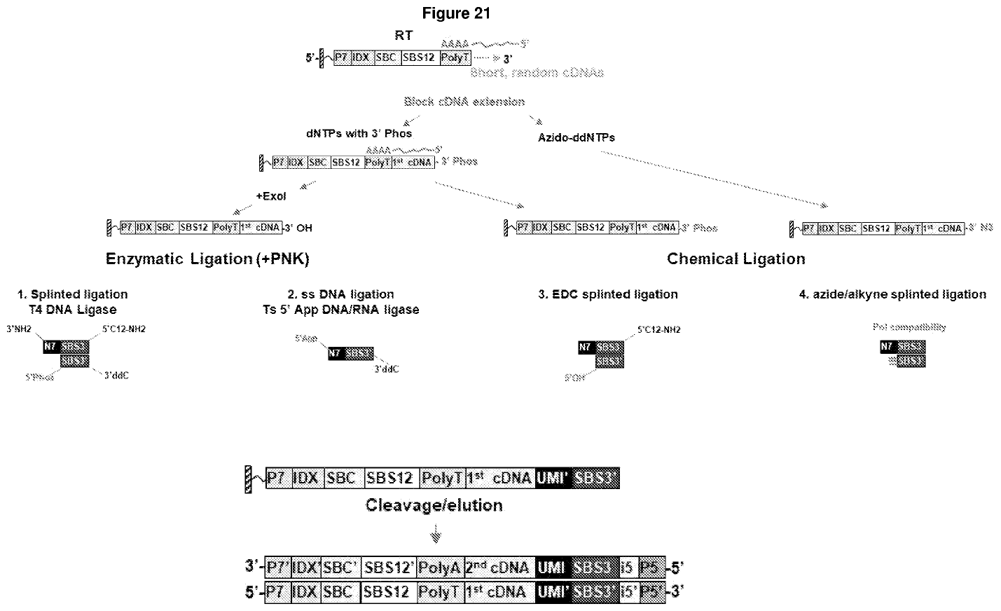

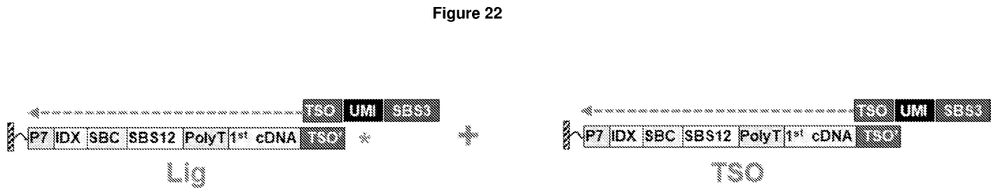

Generation of second complementary strands may be performed “on surface” or “off surface”. In some embodiments, for on surface generation of second complementary strands the first complementary strands remain immobilized on the surface while second complementary strands are extended using the first complementary strands as template. See, e.g., FIG. 16, FIG. 19, and FIG. 20. In some embodiments, the second complementary strands are removed (eluted) from the surface, after which the second complementary strands are subjected to indexed PCR for amplification. In some embodiments, for on surface generation of second complementary strands an Exclusion Amplification (ExAmp) mix comprising an adapter-index oligonucleotide is contacted with the first complementary strands on the surface, thereby generating second complementary strands via strand invasion and isothermal amplification. In various embodiments, the Examp mix further comprises a recombinase, a single-strand DNA binding protein (e.g., gp32 ssDNA binding protein), and a polymerase. As with any of the methods of generating second complementary strands described herein, generation of the second complementary strands may subsequently be followed by amplification of the second complementary strands (e.g., by indexed PCR), during which a second clustering primer sequence (e.g., P5) may be added to one or more of the second complementary strands. In some embodiments, the amplifying comprises index PCR during which a first primer hybridizes to the first clustering primer sequence and a second primer hybridizes to the adapter nucleotide sequence, wherein the second primer comprises the second clustering primer sequence. In various embodiments, an indexing sequence (e.g., i5) is also added to one or more of the second complementary strands during amplification. In some embodiments, the second primer further comprises the indexing sequence. Addition of the second clustering primer sequence and optionally the indexing sequence to the one or more of the second complementary strands occurs, in various embodiments, off of the surface (e.g., in solution). The amplification of the second complementary strands may subsequently be followed by sequencing. The sequencing information may subsequently be correlated with a spatial location of the target nucleic acids in the biological sample.

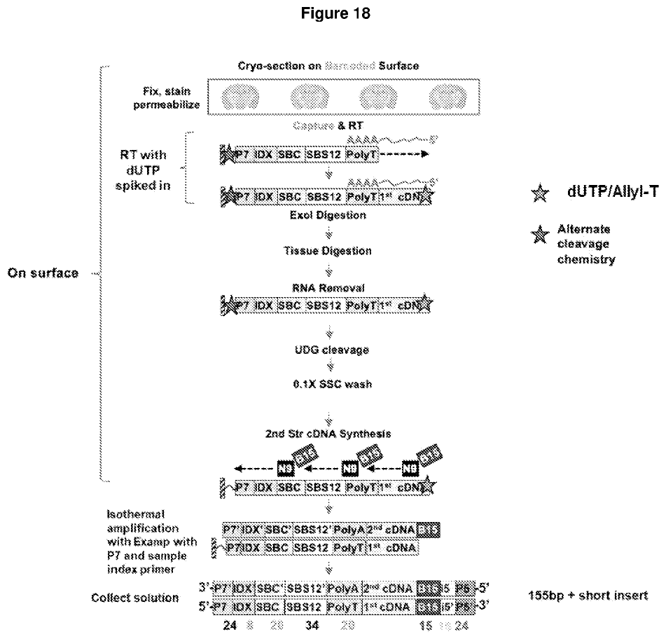

In some embodiments, for off surface generation of second complementary strands formation of the first complementary strands is followed by cleavage of the first complementary strands from the surface, and second complementary strand synthesis is performed off of the surface (e.g., in solution). See, e.g., FIG. 18. In some embodiments, the second complementary strands are then amplified (e.g., via indexed PCR), during which a second clustering primer sequence (e.g., P5) may be added to one or more of the second complementary strands. In various embodiments, an indexing sequence (e.g., i5) is also added to one or more of the second complementary strands during amplification. Addition of the second clustering primer sequence and optionally the indexing sequence to the one or more of the second complementary strands occurs, in various embodiments, off of the surface (e.g., in solution). The amplification of the second complementary strands may subsequently be followed by sequencing. The sequencing information may subsequently be correlated with a spatial location of the target nucleic acids in the biological sample.

In some aspects, a method of preparing an immobilized library of target nucleic acids of a biological sample, comprising providing a surface comprising capture oligonucleotides that hybridize or otherwise associate with the target nucleic acids of the biological sample, extending (e.g., via reverse transcription) the capture oligonucleotides to form first complementary strands of the target nucleic acids, wherein the extending is in the presence of an extension termination moiety, and wherein the extension termination moiety is an allyl-T or a deoxyuridine triphosphate (dUTP), thereby preparing the immobilized library of target nucleic acids. In various embodiments, one or more of the capture oligonucleotides comprises, from 5′ to 3′: (i) a first clustering primer sequence; (ii) a spatial barcode (SBC) sequence; (iii) a first sequencing primer sequence; and (iv) a capture nucleotide sequence. In some embodiments, second complementary strands are generated on the surface, and the second complementary strands are removed (e.g., eluted) from the surface. In some embodiments, second complementary strands are generated on the surface, and the second complementary strands are removed (e.g., eluted) from the surface by ExAmp mix. In some embodiments, the surface is contacted with an exonuclease after the first complementary strands are generated (e.g., to remove unbound surface capture oligonucleotides from the surface), and a plurality of oligonucleotide primers is hybridized to the first complementary strands, wherein each of the plurality of oligonucleotide primers comprises, from 5′ to 3′: (i) an adapter nucleotide sequence (e.g., B15); and (ii) a random nucleotide sequence (comprising, e.g., about 5-10 nucleotides, 7-10 nucleotides, or 9 nucleotides). In some embodiments, the plurality of oligonucleotide primers is then extended, thereby generating one or more second complementary strands comprising the adapter nucleotide sequence at a terminus. In some embodiments, the one or more second complementary strands is removed from the surface and amplified. In further embodiments, the amplifying is performed in the presence of an Exclusion Amplification (ExAmp) mix, wherein the ExAmp mix comprises a primer comprising the clustering primer sequence. In various embodiments, the ExAmp mix further comprises a recombinase, a single-strand DNA binding protein (e.g., gp32 ssDNA binding protein), and a polymerase. In some embodiments, one or more of the plurality of capture oligonucleotides is immobilized on the surface through a cleavage site. In some embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In various embodiments, the cleavage site is cleaved after the capture nucleotide sequence has been extended and the first complementary strands have been formed. In some embodiments, the extension termination moiety is a deoxyuridine triphosphate (dUTP) and the method further comprises contacting the surface with a uracil-DNA glycosylase (UDG). In some embodiments, the surface is contacted with the UDG after the first complementary strands are generated and before the second complementary strands are generated. In some embodiments, the extension termination moiety is an allyl-T and the method further comprises contacting the surface with a universal cleavage mix (UCM) (see, e.g., International Application Publication Number WO 2019/222264, incorporated by reference herein in its entirety, for discussion of cleavage mixes). In some embodiments, contacting the surface with a universal cleavage mix (UCM) occurs before the plurality of oligonucleotide primers is hybridized to the first complementary strands. In some embodiments, the second complementary strands are generated off of the surface (e.g., in solution).

In some aspects, a method of preparing an immobilized library of target nucleic acids of a biological sample, comprising providing a surface comprising capture oligonucleotides that hybridize or otherwise associate with the target nucleic acids of the biological sample, extending (e.g., via reverse transcription) the capture oligonucleotides to form first complementary strands of the target nucleic acids, wherein the extending is in the presence of an extension termination moiety, and wherein the extension termination moiety is a dideoxynucleoside triphosphate (ddNTP), thereby preparing the immobilized library of target nucleic acids. In some embodiments, one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence. In some embodiments, second complementary strands are generated on the surface, and the second complementary strands are removed (e.g., eluted) from the surface. In some embodiments, second complementary strands are generated on the surface, and the second complementary strands are removed (e.g., eluted) from the surface by Examp mix. In some embodiments, the second complementary strands are generated off of the surface (e.g., in solution). More specifically, in some embodiments, the surface is contacted with an exonuclease, and a plurality of oligonucleotide primers is hybridized to the first complementary strands, wherein each of the plurality of oligonucleotide primers comprises, from 5′ to 3′: (i) an adapter nucleotide sequence (e.g., B15); and (ii) a random nucleotide sequence (comprising, e.g., about 5-10 nucleotides, 7-10 nucleotides, or 9 nucleotides). Next, the plurality of oligonucleotide primers is extended, thereby generating one or more second complementary strands comprising the adapter nucleotide sequence at a terminus. In various embodiments, following removal of the one or more second complementary strands from the surface, the one or more second complementary strands are amplified via, e.g., indexed PCR. The amplification of the one or more second complementary strands results in addition of a second clustering primer sequence (e.g., P5) to the one or more of the second complementary strands. In various embodiments, an indexing sequence (e.g., i5) is also added to the one or more second complementary strands during amplification. Thus, in some embodiments, amplification of the one or more second complementary strands results in addition of a second clustering primer sequence (e.g., P5) and an indexing sequence (e.g., i5) to the one or more second complementary strands. In various embodiments, removing the one or more second complementary strands from the surface is performed in the presence of an Exclusion Amplification (ExAmp) mix, wherein the ExAmp mix comprises a primer comprising the first clustering primer sequence. In some embodiments, one or more of the plurality of capture oligonucleotides is immobilized on the surface through a cleavage site. In some embodiments, the cleavage site is an enzymatic cleavage site. In various embodiments, the enzymatic cleavage site comprises a restriction enzyme site, a uracil, an 8-oxoguanine, or a combination thereof. In some embodiments, the cleavage site is a chemical cleavage site. In various embodiments, the cleavage site is cleaved after the capture nucleotide sequence of the hybridized capture oligonucleotides is extended. In some embodiments, the cleavage site is cleaved after the one or more second complementary strands is generated. In some embodiments, the ddNTP comprises a first click chemistry handle. In further embodiments, after the capture nucleotide sequence of the hybridized capture oligonucleotides is extended, the surface is contacted with an adapter oligonucleotide comprising a second click chemistry handle capable of crosslinking to the first click chemistry handle, thereby ligating the adapter oligonucleotide to the first complementary strands. Any click chemistry moiety is contemplated for use in the methods of the disclosure. In some embodiments, the adapter oligonucleotide further comprises a second sequencing primer sequence. In some embodiments, the first click chemistry handle is an azide, a tetrazine, a strained alkene, or an alkyne. In some embodiments, the second click chemistry handle is an azide, a tetrazine, a strained alkene, or an alkyne.

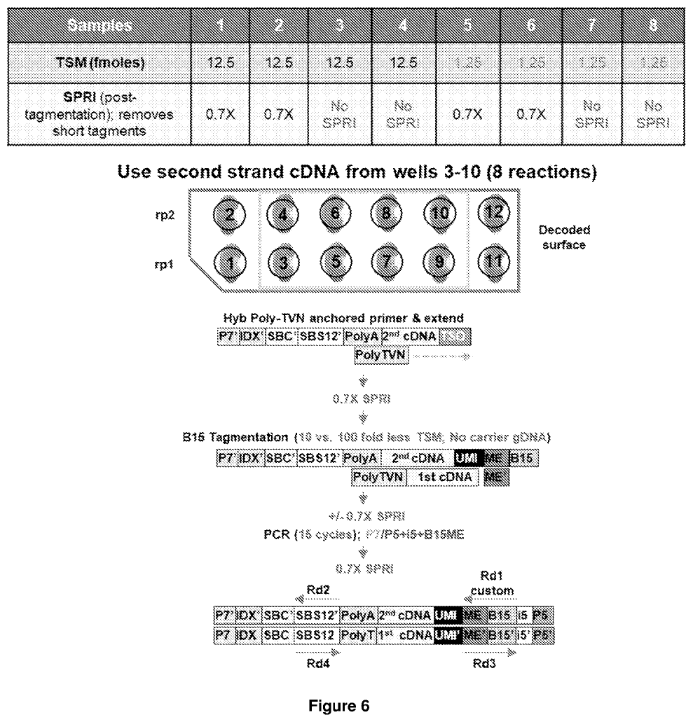

In some aspects, a method of preparing an immobilized library of target nucleic acids of a biological sample, comprising providing a surface comprising capture oligonucleotides that hybridize or otherwise associate with the target nucleic acids of the biological sample, extending (e.g., via reverse transcription) the capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate, thereby preparing the immobilized library of target nucleic acids. See, e.g., FIG. 6. In various embodiments, one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence. Next, the surface is contacted with an exonuclease and ligation is subsequently performed to ligate an adapter oligonucleotide to the first complementary strands. The adapter oligonucleotide comprises (i) an adapter nucleotide sequence (e.g., B15); and (ii) a random nucleotide sequence (comprising, e.g., about 5-10 nucleotides, 7-10 nucleotides, or 9 nucleotides). In some embodiments, the adapter oligonucleotide further comprises a second oligonucleotide that is hybridized to the adapter nucleotide sequence. In some embodiments, the adapter nucleotide sequence comprises a second sequencing primer sequence. In various embodiments, ligation occurs through a splinted ligation of the adapter oligonucleotide to the first complementary strands. In various embodiments, ligation is performed using a T4 DNA ligase. In some embodiments, the ligating occurs through a single-stranded DNA ligation of the adapter oligonucleotide to the first complementary strands. In related embodiments, the ligase enzyme is a DNA/RNA ligase. More generally regarding the ligation, in some embodiments the adapter oligonucleotide further comprises a second oligonucleotide that is hybridized to the adapter nucleotide sequence; these adapter oligonucleotides comprising a second oligonucleotide that is hybridized to the adapter nucleotide sequence may be used, e.g., to facilitate splinted ligation. In various embodiments, the ligation is enzymatic ligation. In further embodiments, the enzymatic ligation is splinted ligation. In some embodiments, the enzymatic ligation is single-strand DNA ligation. In some embodiments, the ligation is chemical ligation. In some embodiments, the chemical ligation is splinted ligation. In some embodiments wherein splinted ligation was utilized, second complementary strands are generated on the surface using the adapter nucleotide sequence as a primer sequence. In some embodiments wherein single-strand DNA ligation was utilized, second complementary strands are generated on the surface using a primer that is complementary to the adapter nucleotide sequence. In some embodiments, the second complementary strands are then removed (e.g., eluted) from the surface. In some embodiments, second complementary strands are generated on the surface, and the second complementary strands are removed (e.g., eluted) from the surface by ExAmp mix. In some embodiments wherein splinted ligation was utilized, the second complementary strands are generated off of the surface (e.g., in solution) using the adapter nucleotide sequence as a primer sequence. In some embodiments wherein single-strand DNA ligation was utilized, the second complementary strands are generated off of the surface (e.g., in solution) using a primer that is complementary to the adapter nucleotide sequence. Following ligation of the adapter oligonucleotide to the first complementary strands, the adapter oligonucleotide is extended, thereby generating one or more second complementary strands. The one or more second complementary strands is then removed from the surface and amplified. Amplification is performed, in various embodiments, in the presence of an Exclusion Amplification (ExAmp) mix. In some embodiments, one or more of the plurality of capture oligonucleotides is immobilized on the surface through a cleavage site. In some embodiments, the cleavage site is an enzymatic cleavage site. In further embodiments, the enzymatic cleavage site comprises a restriction enzyme site, a uracil, an 8-oxoguanine, or a combination thereof. In some embodiments, the cleavage site is a chemical cleavage site. In various embodiments, the cleavage site is cleaved after the capture nucleotide sequence of the hybridized capture oligonucleotides is extended. In some embodiments, the cleavage site is cleaved after the surface is contacted with a ligase enzyme to ligate the adapter oligonucleotide to the first complementary strands.

In some aspects, a method of preparing an immobilized library of target nucleic acids of a biological sample, comprising providing a surface comprising capture oligonucleotides that hybridize or otherwise associate with the target nucleic acids of the biological sample, extending (e.g., via reverse transcription) the capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate or a dideoxynucleoside triphosphate (ddNTP) comprising a first click chemistry handle, thereby preparing the immobilized library of target nucleic acids. See, e.g., FIG. 6. In various embodiments, one or more of the plurality of capture oligonucleotides comprises, from 5′ to 3′: (i) a first clustering primer sequence; (ii) a spatial barcode (SBC) sequence; (iii) a first sequencing primer sequence; and (iv) a capture nucleotide sequence. In some embodiments, the extension termination moiety is the deoxynucleoside triphosphate (dNTP) comprising a 3′ phosphate. In these embodiments, an adapter oligonucleotide is then chemically ligated to the first complementary strands through a crosslinking group, wherein the adapter oligonucleotide comprises, from 5′ to 3′: (i) an adapter nucleotide sequence (e.g., B15); and (ii) a random nucleotide sequence (comprising, e.g., about 5-10 nucleotides, 7-10 nucleotides, or 9 nucleotides). In some embodiments, the adapter oligonucleotide further comprises a second oligonucleotide that is hybridized to the adapter nucleotide sequence. In some embodiments, the adapter nucleotide sequence comprises a second sequencing primer sequence. In various embodiments, the crosslinking group is a carboxyl-to-amine reactive group, a BCN-azide reactive group, a DBCO-azide reactive group, a Tetrazine-TCO reactive group, or a combination thereof. In some embodiments, the extension termination moiety is the dideoxynucleoside triphosphate (ddNTP) comprising the first click chemistry handle. In these embodiments, an adapter oligonucleotide is then ligated to the first complementary strands through click chemistry, wherein the adapter oligonucleotide comprises, from 5′ to 3′: (i) an adapter nucleotide sequence; and (ii) a random nucleotide sequence, and wherein the adapter oligonucleotide further comprises a second oligonucleotide that is hybridized to the sequencing primer sequence, wherein the second oligonucleotide comprises a second click chemistry handle. In some embodiments, the adapter nucleotide sequence comprises a second sequencing primer sequence. In various embodiments, the first click chemistry handle is an azide, a tetrazine, a strained alkene, or an alkyne. In further embodiments, the second click chemistry handle is an azide, a tetrazine, a strained alkene, or an alkyne.

Methods of the disclosure further provide, in various embodiments, that the biological sample is digested. The digestion of the biological sample can occur, in various embodiments, after generation of the first complementary strands. In some embodiments, digestion of the biological sample occurs after generation of the first complementary strands but prior to generation of second complementary strands. The disclosure also provides methods in which the target nucleic acids (e.g., RNA) are removed from the surface. Removal of target nucleic acids from the surface can occur, in various embodiments, after generation of the first complementary strands. In some embodiments, removal of the target nucleic acids occurs after generation of the first complementary strands but prior to generation of second complementary strands. Removal of the target nucleic acids from the surface is achieved, in various embodiments, by changing a condition. In further embodiments, the condition is temperature, pH, formamide concentration, or a combination thereof.

Aspects of the disclosure include those in which a plurality of capture oligonucleotides is immobilized on a surface. The capture oligonucleotides, in various embodiments, hybridize to target nucleic acids of a biological sample. In some embodiments, each of the plurality of capture oligonucleotides comprises the same capture nucleotide sequence. In further embodiments, the plurality of capture oligonucleotides comprises multiple, different capture nucleotide sequences. In still further embodiments, the multiple, different capture nucleotide sequences comprise one or more gene-specific capture sequences, one or more universal capture sequences, or a combination thereof. In various embodiments, the capture nucleotide sequence is a poly-T sequence, a poly-A sequence, a gene-specific capture sequence, or a universal capture sequence. In further embodiments, the universal capture sequence is a random nucleotide sequence or a non-self complementary semi-random sequence. Aspects of the disclosure also include those in which capture nucleotide sequences are extended following hybridization of the capture oligonucleotide to the target nucleic acid. In some embodiments, the extending of the capture nucleotide sequence is carried out using a reverse transcriptase. In some implementations of a method of the disclosure, the target nucleic acids are polyadenylated prior to hybridization of the target nucleic acids to the capture nucleotide sequences. In some embodiments, the target nucleic acids are polyadenylated using a poly(A) polymerase. In further embodiments, the target nucleic acids are polyadenylated using chemical ligation or enzymatic ligation.

In various embodiments, a primer (e.g., an oligonucleotide primer that is hybridized to the first complementary strands and then extend) used in a method of the disclosure is used at a concentration in the range of 0.1 μM to 100 μM, 1 μM to 100 μM or 3 μM to 75 μM or 5 to 50 M. In some embodiments, the primer is used at a concentration of 0.25 μM, 0.5 μM or 1.1 μM or 2.2 μM. In still further embodiments, the primer is used at a concentration of 1 μM 5 μM, 10 μM, 25 μM or 50 μM. In various embodiments, such a primer is a P5 primer, a P5′ primer, a P7 primer, or a P7′ primer.

### Kits

Kits and articles of manufacture are also contemplated herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. For example, the container(s) can comprise one or more spatially addressable probes disclosed herein, optionally in a composition or in combination with another agent (e.g., an array, a beadchip) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use with the spatially addressable probes described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific spatial-omic applications. The label can also indicate directions for use of the contents, such as in the methods described herein.

The following examples are intended to illustrate but not limit the disclosure. While they are typical of those that might be used, other procedures known to those skilled in the art may alternatively be used.

### EXAMPLES

### Example 1

Fresh frozen tissue was sectioned and fixed to a polyT barcoded, capture flow cell. Tissues were methanol-fixed at −20° C. for 30 minutes, after which they were stained with hematoxylin and eosin, air-dried, and imaged with an optical microscope. The substrate was then placed in a proprietary device with sealable wells, which allowed heated incubations on a thermal cycler. Each well had an approximate surface area of 28 mm\hat{ }2 overlying each tissue section.

The samples were incubated at 53° C. for 1 hour in first strand cDNA synthesis mix with a Rd1-containing template switch oligonucleotide. The wells are then washed 3 times with water. NaOH is added to the wells and incubated at room temp for 5 minutes three times.

The flow cell was assembled into a gasket that created individual sample wells over the tissue section. Wells were coated with 0.1×SSC/RNase Inhibitor and the solution was removed. 25 ul pre-warmed permeabilization mix (0.1% pepsin, 0.1N HCl) was added to the wells and the flow cell was incubated for 7 minutes at 37° C. followed by three room temperature washes in spatial wash buffer. The permeabilization mix was then removed and 25 ul 1× RT buffer with RNase Inhibitor was added to the wells. The buffer was then removed and 25 ul cDNA synthesis mix (reverse transcription (RT) enzyme, reducing agent, TSO primer, RT reagent, water) was added to the wells. The flow cell was incubated for 1 hour at 53° C. in first strand cDNA synthesis mix with a Rd1-containing template switch oligonucleotide. The solution was then discarded from the wells and washed three times with water. 25 ul of 100% formamide was added to the wells and the flow cell was incubated at 80° C. for 10 minutes.

The mRNA elution was stored at −80° C. for RT-qPCR QC. The wells were then washed three times with water. 25 ul 0.08M KOH was added to the wells and the flow cell was incubated at room temperature for 5 minutes. The solution was discarded, and the wells were washed once with 50 ul Buffer EB (Qiagen). Second strand synthesis was then performed by adding 25 ul second strand synthesis mix (2^{nd} strand reagent, TSO primer, 2^{nd} strand enzyme) to the wells and the flow cell incubates at 65° C. for 15 minutes. The solution was then discarded, and the wells were washed with 50 ul Buffer EB. The buffer was then discarded and 25 ul 0.08M KOH was added to the wells and the flow cell was incubated at room temperature for 10 minutes.

The eluted cDNA in KOH was moved to strip tubes and neutralized with 3.6 ul Tris (1M, pH 7.0). Poly-TVN extension mix (Illumina ASM strand displacing extension mix) was then added to the neutralized cDNA and the mixture was subjected to the following parameters on a thermocycler: 37° C. for 10 min, 60° C. for 10 min, and hold at 4° C. The extended product was 0.7×SPRI-purified and eluted in 12 ul water. Tagmentation using Illumina Surecell protocol was then performed (with diluted transposome). Tagmentation reaction mix (Tagment enzyme, Tagment Buffer) was added to the purified sample and incubates at 55° C. for 5 minutes. Tagmentation was stopped with 10 ul Tagment stop buffer and 5-minute incubation at room temperature. Index PCR mix (Tagmentation PCR Mix, P7 primer, N5XX primer) was then added to the sample and the following parameters were used: 95° C. for 30s, followed by 15 cycles of 95° C. for 10s, 60° C. for 45s, 72° C. for 60s, then a final extension at 72° C. for 5 minutes and hold at 4° C. The samples were then purified with 1×SPRI and quantified for sequencing. Libraries were sequenced per NovaSeq 6000 S4 flowcell with read structure: 100 bases Read 1 (custom primer), 28 bases Index Read 1, 8 bases Read 2 (FIG. 2).

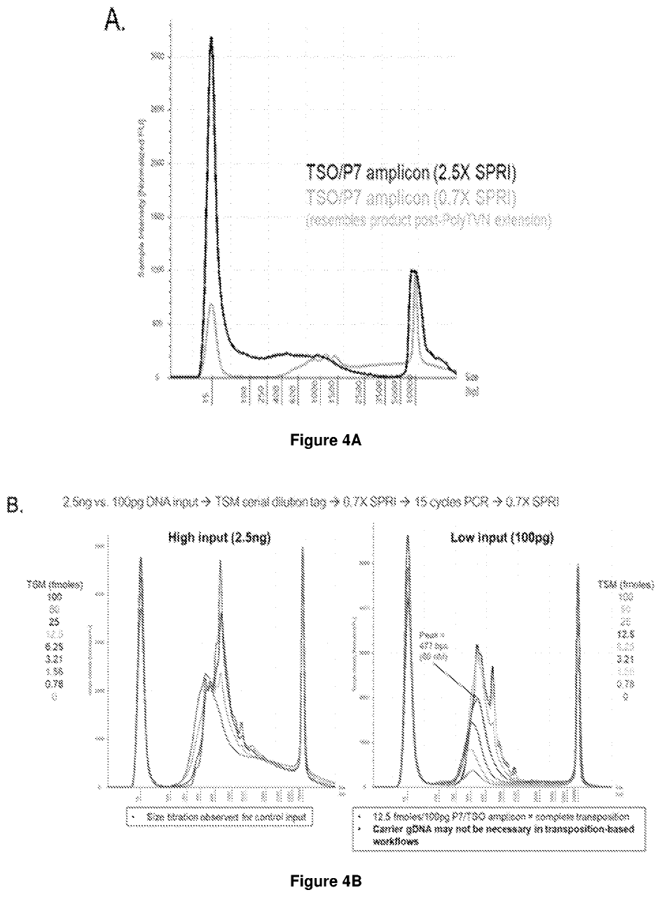

Referring to FIG. 4, data generated from the TSO-TAG process described above is shown. An amplicon was generated from TSO-TAG second strand cDNA with P7 and TSO primers and 0.7×SPRI purified to mimic optimized PolyTVN-extended product. This amplicon was used for in-solution tagmentation evaluation as shown in FIG. 4A. Referring to FIG. 4B, a high input P7/TSO amplicon (2.5 ng) which serves as a control (optimal input for tagmentation with Illumina's Surecell library Prep Kit), versus low input amplicon (100 pg) was tagmented with a 2 fold serial dilution of Surecell transposome (B15 transposome diluted in Illumina's standard storage buffer) starting at 100 fmoles to 0 fmoles. Tagmentation was performed following Illumina's standard Surecell tagmentation protocol. Fragment size titration was observed. Libraries were then 0.7×SPRI purified. 15 cycles of PCR with B15-index-P5 and P7 was performed and then the product was 0.7×SPRI purified. Libraries were then run on a D5000 DNA screen tape on an Agilent Tapestation. A sequencing library with a peak at 477 bps when transposed with 12.5 moles B15 transposome can be observed with low input tagmentation (60 nM yield). This suggests low input tagmentation can generate desirable sequencing libraries (desired size range 200-800 bps, with high yield), as shown by readout on D5000 DNA screen tape on Tapestation, and addition of carrier gDNA to prevent over-tagmentation is not necessary.

Referring to FIG. 6, different conditions were tested to assess how process parameters affect the final library preparation. 12.5 fmols B15 Tn5 transposase and 1.25 fmoles B15 Tn5 transposase in the tagmentation mix were tested as well as purification with 0.7×SPRI post-tagmentation pre-index PCR vs no SPRI. The gene mapping number for these different conditions is shown in the table below, along with a comparison to a commercially available library preparation method-Visium (10× Genomics). The process in accordance with the disclosure had improved mapped gene as measured in transcripts per million (TPM) as compared to the commercial product.

| Well | TSM (fmoles) | SPRI pre-PCR | TPM (>1) |
|---|---|---|---|
| 3 | 12.5 | + | 13372 |
| 4 | 12.5 | + | 13400 |
| 5 | 12.5 | − | 13190 |
| 6 | 12.5 | − | 13138 |
| 7 | 1.25 | + | n/a |
| 8 | 1.25 | + | 12805 |
| 9 | 1.25 | − | 12529 |
| 10 | 1.25 | − | 13103 |
| Commercial Product | | | 10477 |

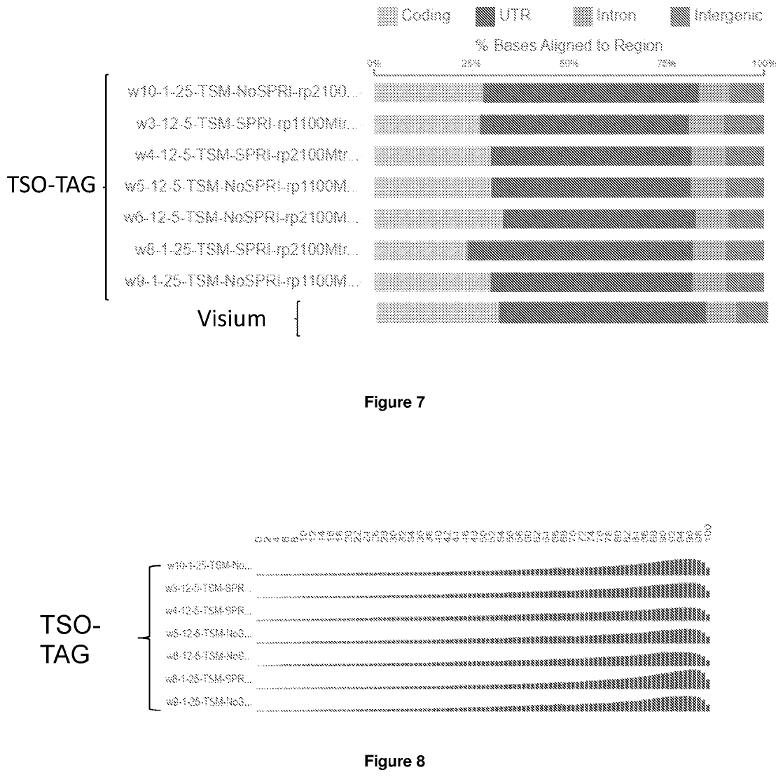

Coding alignment data was generated for all the TSO-TAG libraries using Illumina's RNA-seq alignment software (v2.0.2) on BaseSpace. Referring to FIG. 7, the alignment distribution for the method of the disclosure was similar to the commercially available Visium process, as well. Transcript coverage sequencing data was generated for all the TSO-TAG libraries using Illumina's RNA-seq alignment software (v2.0.2) on BaseSpace. FIG. 8 shows the transcript coverage of the TSO-TAG method. The 3′ coverage bias was observed to be shifted slight with the transposition based library preparation method of the disclosure.

### Example 2

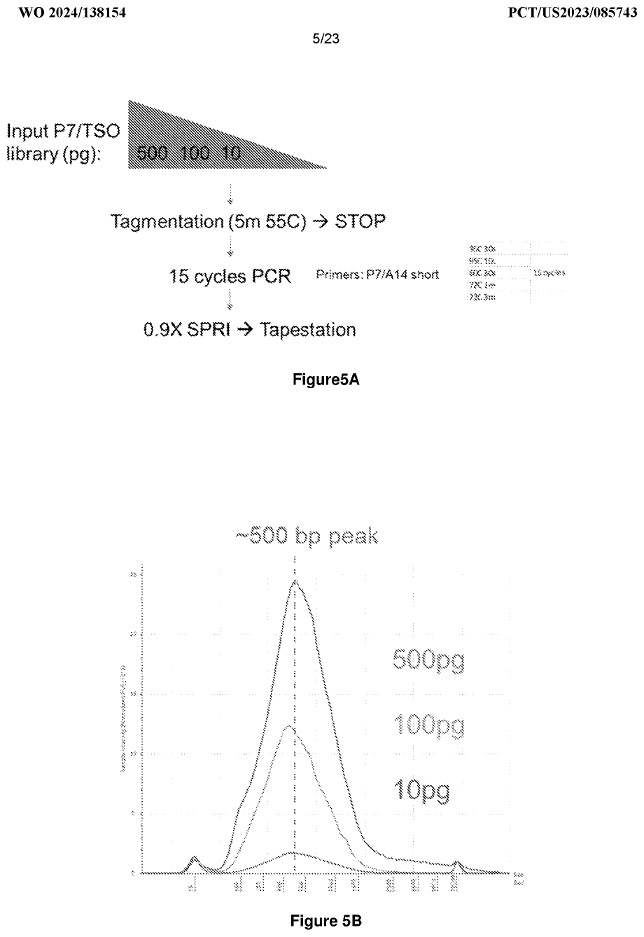

The library preparation process as described in Example 1 was performed except transposition was performed on beads. A14 transposome beads were formed and stored in 15% glycerol storage buffer. 500 pg, 100 pg, and 10 pg P7/TSO amplicons were transposed with A14 beads. The reaction was stopped with buffer and amplified with P7/A14 short for 15 cycles using the same thermocycling parameters described in Example 1. Libraries were 0.9×-SPRI-purified and run on HD500 Tapestation. FIG. 5A is a schematic illustration of the process and FIG. 5B is a graph of the library fragment size, which was uniform for 500 pg, 100 pg, and 10 pg.

The TSO-TAG workflow can also be performed in a “one-pot” reaction when using a biotinylated TSO second strand oligo in second strand cDNA synthesis. TSO-TAG is performed in the same way outlined in FIG. 1. After elution and neutralization of second strand cDNA, the second strand cDNA can be hybridized to streptavidin beads and subsequent steps (Poly-TVN extension, tagmentation, wash and PCR) can be performed on-beads with simple wash steps on magnet (FIG. 9A).

TSO-TAG can be performed on-surface (FIG. 9B). After RNA removal, 3′-blocked SBS12′-PolyA hybridizes to cDNA. TSO complement oligo then gap fills to 5′ polyA region (using non-strand displacing polymerase). The SBS12′-PolyA region is then melted off with heat. On-surface tagmentation is then performed (A14 transposome) to introduce UMI and add adapter. Proprietary extension mix is then added to the surface to add the P7 and A14 adapters to the cDNA. The second strand product is then eluted from the surface and subject to indexed PCR.

Various library prep methods including transposition are also contemplated in addition to TSO-TAG (SBC-TAG and UMI-TAG) (FIG. 10). In one method, SBC-TAG and UMI-TAG uses random priming (no template switch) with N9-SBS3 randomers and a second strand mix for second strand cDNA synthesis. Second strand cDNA is then eluted in a proprietary buffer and global pre-amplification is used to build redundancy prior to transposition using the same cycling parameters described in FIG. 1. Amplified libraries are SPRI purified and subject to tagmentation following the protocol described in FIG. 4B. To generated UMI information, libraries are also amplified with P7 and D5XX primers. This approach leaves the barcode information double-stranded, which can allow for transposition on capture sequence region.

### Example 3

The following experiment was performed to show feasibility of transposition of amplified TSO library. 0.6×SPRI-purified second strand cDNA (generated in method described in FIG. 1) was subject to transposition with Nextera XT transposome (1 ng library input), Surecell transposome (3 ng library input), or custom A14ME transposome (3 ng library input). Tagmentation was performed following Illumina's standard tagmentation protocol. Nextera libraries were amplified with UD Index primers using the following cycling parameters: 72° C. for 3 minutes, 95° C. for 30 seconds, followed by 12 cycles of 95° C. for 10 seconds, 55° C. for 30 seconds, and 72° C. for 30 seconds, with a final extension at 72° C. for 5 minutes and a hold at 10° C. Surecell libraries were amplified with P7/N5XX primers using the following cycling parameters: 95° C. 30 seconds, followed by 12 cycles of 95° C. for 10 seconds, 60° C. for 45 seconds, and 72° C. for 60 seconds, with a final extension at 72° C. for 5 minutes and a hold at 10° C. Custom A14ME libraries were amplified with P7/A14-index-P5 primers using the following cycling parameters: 95° C. 30 seconds, followed by 12 cycles of 95 C for 10 seconds, 60° C. for 45 seconds, and 72° C. for 60 seconds, with a final extension at 72° C. for 5 minutes and a hold at 10° C. Libraries were 0.6×SPRI-purified and sequenced using the same parameters described in FIG. 2.

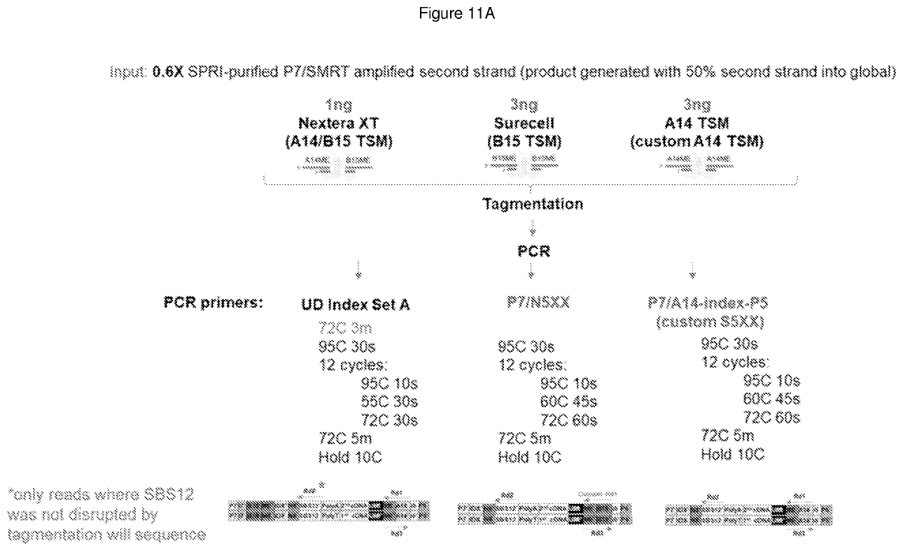

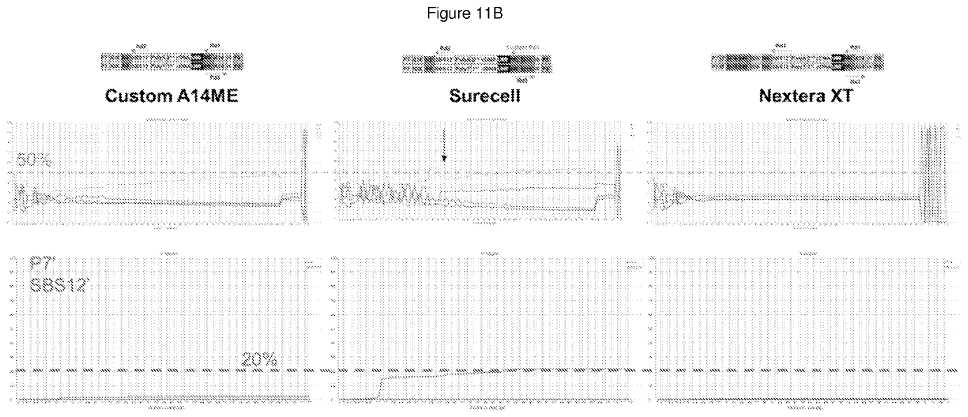

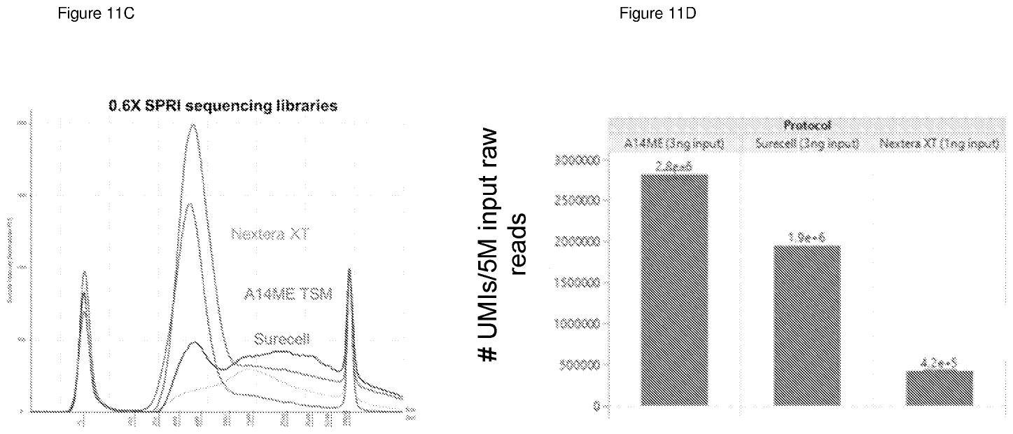

Using a sequencing analysis pipeline, base composition plots of Read 1 were determined (FIG. 11B). A14ME custom libraries showed little to no amplification in adapter region of the amplified library. No transposition in adapter region for Nextera libraries due to A14/B15 transposome.

0.6×SPRI-purified libraries were run on a HSD5000 DNA Screentape on an Agilent Tapestation to determine library concentration for sequencing (FIG. 11C). A14ME libraries show similar library conversion to Nextera libraries with Surecell being under-tagmented.

Using a sequencing analysis pipeline, the # of UMIs per 5M input raw reads was determined (FIG. 11D). Highest UMI count was determine for the A14ME-transposed libraries. Low number of UMIs determine for Nextera XT due to A14/B15 transposome.

### Example 4

Spatial transcriptomics enables highly multiplexed in situ gene expression profiling within complex tissues. A key challenge in achieving single-cell resolution is assay sensitivity, in which tissue RNAs are converted into sequence-ready libraries. Described herein is a method which combines on-surface template-switching, single-stranded enzymatic ligation (TSO-LIG) and isothermal amplification to efficiently convert captured tissue RNAs, e.g., mRNAs, into spatially barcoded libraries.

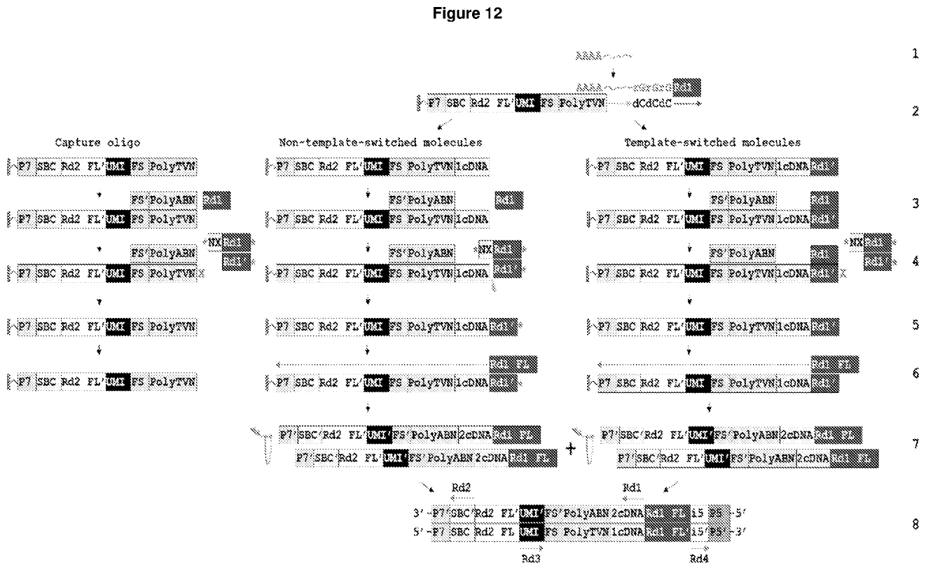

An exemplary workflow is set out in FIG. 12.

In steps 1-2 of the workflow tissue sections are permeabilized to release RNAs, e.g., mRNAs from the sample. Released mRNAs are then captured by anchored polyTVN strands that are immobilized on a solid substrate. The anchored strands are subsequently converted to first strand cDNA via a template switching reverse transcriptase. During cDNA synthesis a template-switching oligo (TSO), for example encoding a partial read 1 sequencing adapter (Rd1′) is appended, e.g., by hybridization, to the 3′ end of the first strand cDNA. In this example, the capture oligo design consists of six components as follows: a) a 5′ P7 sequence (for clustering), b) a randomized spatial barcode (SBC, which encodes unique positional information for transcripts), c) a full-length read 2 adapter sequence (Rd2 FL), for decoding the SBC and the surface UMI, d) a unique molecular identifier (UMI), which provides a discrete barcode for each captured mRNA transcript, e) a fixed sequence (FS), for quality control, and f) a poly T capture sequence with a 3′ VN terminus to anchor captured mRNAs at the 3′ UTR.

After exonuclease I treatment (to remove all single-stranded capture oligos) and RNA removal (to remove bound RNA and the TSO), an oligo ligation blocker is hybridized (step 3) to prevent template-switched molecules from ligating to the Rd1 adapter (X) during the subsequent enzymatic ligation step. The ligation blocker is an oligo complement of the Rd1′ sequence. An alternative strategy to neutralize surface capture oligos is to hybridize a complementary oligo blocker to generate a double-stranded terminus at the 3′ end of the capture oligo to prevent ligation.

During the single-stranded ligation step (step 4), a splinted Rd1 adapter is appended to all cDNA molecules that were not template-switched. The splinted adapter consists of two parts: a single-stranded splint consisting of random bases (NX), with a blocking group at the 3′ end (*) and a double-stranded partial Rd1 adapter sequence containing blocking groups (*) at the ends of both strands furthest from the splint. The adapter blocking groups prevent adapter self-ligation. cDNA UMIs could also be incorporated in both TSO and adapter designs to aid with downstream analyses of the RNA sequences. The 5′ end of the Rd1′ sequence contains a phosphate group to enable ligation to the 3′ OH end of captured non-template-switched cDNAs via enzymatic ligation. After alkaline treatment (e.g., 0.08 M KOH or 0.1 N NaOH for five minutes at room temperature) to remove the ligation blocker and the splinted strand of the adapter (step 5), all Rd1′ containing cDNAs undergo on-surface isothermal amplification (step 6). Exponential amplification (ExAmp) occurs through priming of a single full-length Rd1 primer (Rd1 FL) and a surface P7 lawn primer, resulting in elution of amplified second strand cDNA products (step 7). These eluted products are then converted into sequence-ready libraries through indexed PCR during which an i5 index and a clusterable P5 end are appended (step 8). Strategies to normalize the fragment length of either single-stranded or double-stranded cDNA products (including tagmentation) are contemplated, as described in co-owned U.S. Provisional Application No. 63/586,872 (Attny Docket No. IP-2576-P) and U.S. Provisional Application No. 63/477,103 (Attny Docket No. IP-2528-P).

Relative sensitivities for generating second strand cDNA using template switching (TSO), single-stranded splinted ligation (LIG) and both methods combined (TSO+LIG) were determined. Fresh frozen sections (10 μm) from mouse kidney were mounted onto a substrate containing spatially barcoded capture oligonucleotides. Tissues were methanol-fixed at −20° C. for 30 minutes, after which they were stained with hematoxylin and eosin, air-dried and imaged with an optical microscope. The substrate was then placed in a device with sealable wells, which allowed heated incubations on a thermal cycler. Each well had an approximate surface area of 28 mm\hat{ }2 overlying each tissue section enabling 80 uL on-surface reaction volumes. Tissue sections were then permeabilized with a proprietary permeabilization reagent at 37° C. for 7 minutes, followed by three room temperature washes.

For TSO only, samples were incubated overnight at 42° C. in first strand cDNA synthesis mix with a Rd1-containing template switch oligonucleotide; for single-stranded ligation (LIG) only, samples were incubated overnight at 42° C. in first strand cDNA synthesis mix without a Rd1-containing template switch oligonucleotide; for TSO+LIG, samples were incubated overnight at 42° C. in first strand cDNA synthesis mix with a Rd1-containing template switch oligonucleotide, followed by ligation.

Following first strand cDNA synthesis, samples underwent a 45 minute incubation in oligonucleotide digestion mix at 37° C., a tissue removal step in a tissue removal mix at 37° C. for 40 min, an RNA removal step comprising three 5 minute room temperature incubations in a RNA removal solution and finally a single room temperature wash in spatial wash buffer. For samples undergoing ligation, a proprietary ligation blocking mix was added and incubated for 10 minutes at 40° C., followed by a single room temperature wash in spatial wash buffer. A ligation mix (with a Rd1-containing splinted adapter) was then added and incubated for 75 minutes at 37° C., followed by a single room temperature wash in spatial wash buffer. A blocker removal solution was added, incubated for 5 minutes at room temperature, followed by a single room temperature wash in spatial wash buffer.

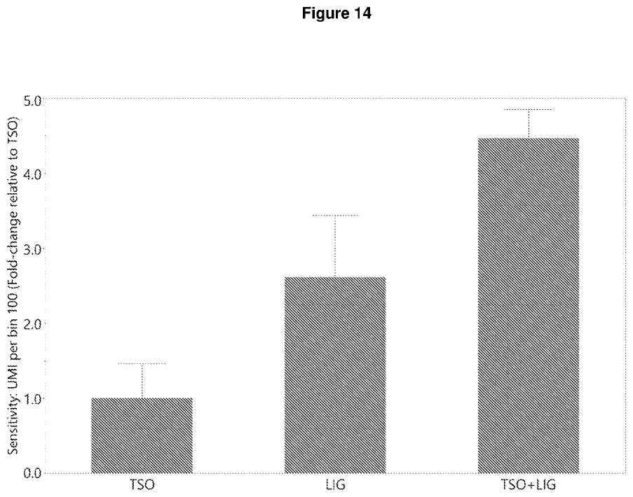

For all three preparations, a proprietary second strand mix was added and incubated for 15 minutes at 65° C. After a single room temperature wash in spatial wash buffer, second strand cDNA was eluted into a PCR tube via two 5 minute room temperature incubations using 22 μL of a elution solution each. A neutralizing solution was added (6 uL) after which a 0.6×SPRI purification was performed. An indexed primer was appended to 10% of the eluted second strand cDNA in a 50 uL PCR reaction (98° C. for 45 seconds, 11 cycles at 95° C. for 30 seconds, 60° C. for 1 minute, 72° C. for 1 minute and a final incubation at 72° C. for 2 minutes) using a 2× Kapa HiFi PCR mix. A 0.7×SPRI purification step was used to clean up the PCR reaction. Sixteen libraries were sequenced per NovaSeq 6000 S4 flowcell with read structure: 100 bases Read 1, 28 bases Index Read 1, 8 bases Read 2. Each sample received approximately 1.2 billion reads after which median UMI counts per 100 μm×100 μm area were extracted using proprietary spatial software and then normalized relative to the TSO condition (FIG. 14).

After captured tissue mRNAs were converted to first strand cDNA on spatially barcoded substrates (similar to that described in FIG. 12), Rd1 adapters were appended either through TSO, LIG or both (TSO+LIG). The samples were then further processed and sequenced as described in FIG. 12. Sensitivity was calculated as median UMIs detected per 100×100 μm and then normalized relative to the TSO condition. FIG. 14 shows that sensitivity is increased in samples where both TSO+ligation methods were used.

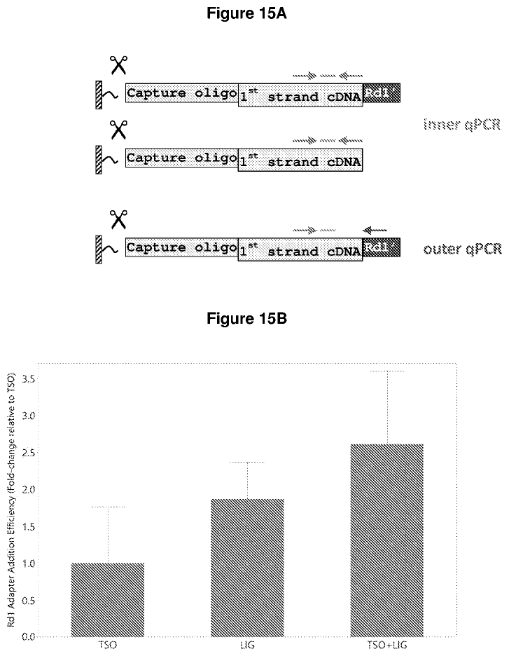

Relative Rd1 adapter addition efficiencies to first strand CDNA using template switching (TSO), single-stranded splinted ligation (LIG) and both methods combined (TSO+LIG) were determined. After second strand cDNA material had been eluted from the spatial substrate, a cleavage mix was added, and samples were incubated at 60° C. for 30 minutes to cleave first strand cDNA from the spatial substrate. First strand cDNA was then cleaved from the substrate and subjected to an inner (5′ gene-specific) and an outer (5′ gene-specific+Rd1) qPCR assay to determine Rd1 adapter addition efficiency (FIG. 15A). qPCR reactions used probe-based assays and a 2× Kapa qPCR mix in a final volume of 10 μL and were cycled as follows: 95 C for 3 minutes, followed by 40 cycles at 95° C. for 15 seconds and 60 C for 90 seconds. Adapter addition efficiency was calculated as 2^{-(Cq inner-Cq outer)} and then normalized relative to the TSO condition. FIG. 15B shows that Rd1 adapter addition efficiency is increased in samples where both TSO+ligation methods were used.

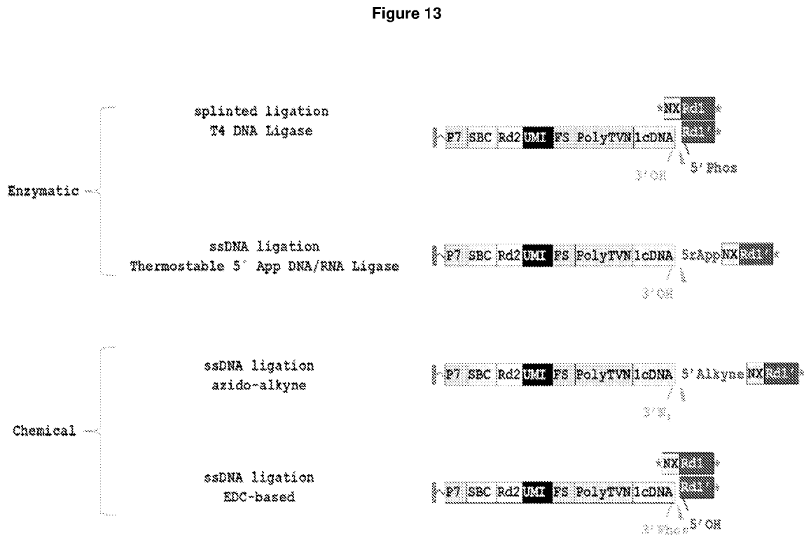

A key step in converting Rd2-containing synthesized cDNA to sequencable libraries is the addition, via single-stranded ligation, of a Rd1 sequence. Exemplary methods to accomplish this are described in FIG. 13.

Ligation options for appending a Rd1 adapter sequence to the 3′ terminus of the first strand cDNA include both enzymatic and chemical methods. Examples of enzymatic methods include: T4 DNA ligase-mediated ligation with a splinted adapter (see FIG. 12) and thermostable 5′ App DNA/RNA ligase-mediated ligation with a synthesized pre-adenylated single-stranded oligo adapter. Chemical ligation methods include: click-chemistry-mediated ligation in which 3′ azido termini are joined to synthesized 5′ alkyne single-stranded oligos or 1-ethyl-3-(3-dimethylaminopropyl) carbodiimide (EDC)-mediated ligation in which cDNAs with 3′ phosphate groups are ligated to splinted adapters with 5′ hydroxyl termini. Incorporation of 3′ azido-ddNTPs or 3′ Phos-dATPs could occur during first strand cDNA synthesis, during which truncated cDNA molecules are generated. Both enzymatic and chemical approaches are compatible with adapters incorporating molecular identifiers, e.g., (NX), to aid in downstream analyses and blocking groups (*) to minimize adapter self-ligation.

### Example 5

Methods of preparing an immobilized library of target nucleic acids of a biological sample (e.g., tissue sample) are provided herein. The methods of the disclosure advantageously normalize library size for on-surface library preparation applications.

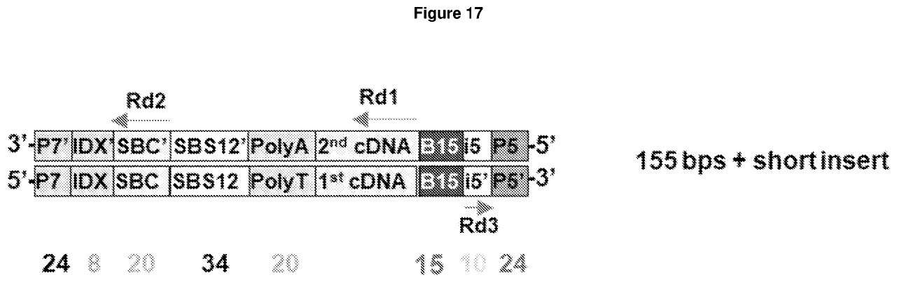

A schematic workflow of a method of the disclosure is shown in FIG. 16. FIG. 16 shows a spatial workflow in which a tissue section is placed on a flow cell (FC) surface, wherein the FC surface comprises a plurality of capture oligonucleotides comprising first clustering primer sequences (for example, P7 primer sequences as shown in FIG. 20). FIG. 16 shows how library fragment size can be controlled with the addition of an optimal concentration of dUTP/allyl-T in the reverse transcription (RT) reaction. This approach is performed by first capturing transcripts on a barcoded surface comprising capture oligonucleotides comprising a polyT capture nucleotide sequence. Reverse transcriptase is initiated with dUTP/allyl-T spiked into the reaction. After RT, the substrate is prepared for UDG/UCM cleavage by performing exonuclease digestion, tissue digestion, and RNA removal steps. As described herein, the tissue digestion step is optional. Cleavage with UDG/UCM is used to generate fragments in which the first dUTP/allyl-T site incorporated will be the 3′ termination site of the cDNA strand. The substrate is then washed and then second strand synthesis is performed with randomer primers comprising an adapter. The randomer sequence will serve as the transcript's UMI. After second strand synthesis, the second cDNA strand is eluted and subjected to indexed PCR and purification before loading onto a sequencer. FIG. 17 depicts the sequencing read structure of the amplified second complementary strands. Read 1 reads into the cDNA; Read 2 reads into the spatial barcode; and Read 3 reads into the sample index. In this example, the total insert size will be 155 bps+short cDNA insert size.

Another schematic workflow of a method of the disclosure is shown in FIG. 18. FIG. 18 shows a spatial workflow in which a tissue section is placed on a flow cell (FC) surface, wherein the FC surface comprises a plurality of capture oligonucleotides comprising first clustering primer sequences (for example, P7 primer sequences as shown in FIG. 20), wherein one or more of the plurality of capture oligonucleotides is immobilized on the surface through a cleavage site. FIG. 18 shows a schematic for library fragment size normalization using dUTP/allyl-T with in-tube second strand synthesis. Adding an alternative cleavage chemistry upstream of the first clustering primer sequence (shown as P7 in FIG. 18) allows for in-solution library preparation post-RT. Two different cleavage chemistries allow for an on-surface wash step to remove unbound cDNA fragments. In this workflow, transcripts are captured on a barcoded surface comprising capture oligonucleotides comprising a polyT capture nucleotide sequence. Reverse transcriptase is initiated with dUTP/Allyl-T spiked into the reaction. After RT, the substrate is prepared for UDG/UCM cleavage by performing exonuclease digestion, tissue digestion, and RNA removal steps. Cleavage with UDG/UCM will generate fragments in which the first dUTP/Allyl-T site incorporated will be the 3′ termination site of the cDNA strand. The substrate is then washed and an additional cleavage step upstream of P7 elutes the cDNA off the surface. Second strand synthesis is performed with randomer primers containing an adapter. The randomer sequence will serve as the transcript's UMI. After second strand synthesis, the second cDNA strand (i.e., second complementary strand) is eluted and subjected to indexed PCR and purification before loading onto a sequencer.

Another schematic workflow of a method of the disclosure is shown in FIG. 19. FIG. 19 shows a spatial workflow in which a tissue section is placed on a flow cell (FC) surface, wherein the FC surface comprises a plurality of capture oligonucleotides comprising first clustering primer sequences (for example, P7 primer sequences as shown in FIG. 20). FIG. 19 shows a schematic of library size normalization with ddNTPs in a reverse transcription reaction. Shorter library fragments can be achieved by adding ddNTPs to the RT reaction. In this workflow, transcripts are captured on a barcoded surface comprising capture oligonucleotides comprising a polyT capture nucleotide sequence. Reverse transcriptase is initiated with ddNTPs spiked into the reaction. After RT, the substrate is prepared for second complementary strand synthesis by performing exonuclease digestion, tissue digestion, and RNA removal steps. Second complementary strand synthesis is performed with randomer primers containing an adapter. The randomer sequence will serve as the transcript's UMI. After second complementary strand synthesis, the second cDNA strand (i.e., second complementary strand) is eluted and subject to indexed PCR and purification before loading onto a sequencer.

Another schematic workflow of a method of the disclosure is shown in FIG. 20. Similar to FIGS. 16, 18, and 19, FIG. 20 shows a spatial workflow in which a tissue section is placed on a flow cell (FC) surface, wherein the FC surface comprises a plurality of capture oligonucleotides comprising first clustering primer sequences (for example, P7 primer sequences as shown in FIG. 20). FIG. 20 shows a schematic in which ExAMP serves as a cDNA elution reagent and builds redundancy pre-sequencing. In some embodiments, when an exonuclease step is added to the workflow post-RT (e.g., to remove unbound surface capture oligonucleotides from the surface) this allows the opportunity to use an on-surface ExAMP reaction to elute barcoded surface-bound libraries and build library yields. After second complementary strand synthesis, ExAMP mix (which comprises P7 and a sample index primer) can be added to the substrate. Isothermal amplification with primer strand invasion is then used to generate indexed P5/P7 libraries. The elution is then transferred to a tube where it is purified and loaded onto a sequencer. Efficient exonuclease digestion of unbound surface oligonucleotides improves the workflow.

Another schematic workflow of a method of the disclosure is shown in FIG. 21. FIG. 21 shows a spatial workflow in which the FC surface comprises a plurality of capture oligonucleotides comprising first clustering primer sequences (for example, P7 primer sequences as shown in FIG. 21). FIG. 21 depicts how cDNA fragments can be shortened with 3′ phosphate dNTPs or azido-ddNTPs. First, 3′Phos dNTPs are added to the RT reaction. The substrate can then be treated with an exonuclease, thereby converting the 3′ phosphate to OH, allowing for enzymatic ligation of UMI and adapter sequence (splinted ligation with ssDNA ligation) with PNK. Additionally, chemical ligation with a water-soluble carbodiimide (EDC) can be performed. In further embodiments, azido-ddNTPs are added to the RT reaction, followed by an azide/alkyne splinted ligation to add a UMI and an adapter sequence.

The foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In the case of conflict, the present description, including definitions, will control.

Throughout the specification, where the compounds, compositions, methods, and/or processes are described as including components, steps, or materials, it is contemplated that the compounds, compositions, methods, and/or processes can also comprise, consist essentially of, or consist of any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

What is claimed is:

1. A method for preparing an RNA sequence library, comprising:
mounting a tissue sample on a substrate comprising a plurality of capture oligonucleotides, wherein the capture oligonucleotides comprise a polyT sequence and library barcode information comprising a spatial barcode sequence (SBC);
capturing polyadenylated mRNA transcripts on the substrate with the capture oligonucleotides;
contacting the substrate with a first strand synthesis mix comprising a reverse transcriptase and a template switch oligonucleotide (TSO) under conditions to generate a first strand comprising a first cDNA complementary to the polyadenylated mRNA transcripts, and a TSO complement hybridized to the 5′ end of the first cDNA, wherein the reverse transcriptase incorporates untemplated cytosine nucleotides at the 5′ end of the first cDNA and the TSO comprises a sequence that hybridizes to the untemplated cytosine nucleotides and the reverse transcriptase extends to generate the TSO complement, as a compliment of the TSO, at the 5′ end of the first cDNA;
eluting the polyadenylated mRNA transcripts from the substrate;
contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial barcode sequence complement (SBC′) that is complementary to the spatial barcode sequence (SBC);
eluting the second strand;
contacting the second strand with a Poly-TVN extension mix comprising a Poly-TVN primer and extending the Poly-TVN primer using the second strand as a template to generate a double-stranded product while maintaining a single-stranded 3′ region containing the library barcode information;
contacting the double stranded product with a transposome under conditions to tagment the double stranded product to form a tagmented product comprising a unique molecular identifier (UMI) and PCR adapter; and
amplifying the tagmented product using index PCR to generate the library.

2. The method of claim 1, wherein the TSO primer in the second strand synthesis mix is a biotinylated TSO primer such that the second strand comprises a biotinylated TSO.

3. The method of claim 2, further comprising contacting the eluted second strand comprising the biotinylated TSO with a functionalized bead.

4. The method of claim 3, wherein the functionalized bead is a streptavidin bead.

5. The method of any one of the preceding claims, wherein the second strand is neutralized after elution and prior to contacting with the Poly-TVN extension mix.

6. The method of any one of the preceding claims, wherein contacting the first strand with the second strand synthesis mix comprises incubating the first strand with the second strand synthesis mix for an incubation time of less than 2 hours.

7. The method of claim 6, wherein the incubation time is about 10 minutes to about 60 minutes.

8. The method of claim 7, wherein the incubation time is about 15 minutes to about 30 minutes.

9. The method of any one of the preceding claims, wherein the transposome is A14 transposome or B15 transposome.

10. The method of any one of the preceding claims, wherein the transposome is a transposome modified bead.

11. The method of any one of the preceding claims, comprising purifying the double stranded product before tagmentation.

12. The method of any one of the preceding claims, comprising purifying the double stranded product after index PCR.

13. The method of any one of the preceding claims, wherein contacting the second strand with the Poly-TVN extension mix comprising incubating the second strand with the Poly-TVN extension mix at a first temperature and first time, at a second temperature higher than the first temperature for a second time, and holding at a hold temperature.

14. The method of claim 13, wherein the first temperature is about 25° C. to about 37° C., the first time is about 10 minutes to about 30 minutes.

15. The method of claim 13 or 14, wherein the second temperature is about 60° C. to about 65° C., the second time is about 10 minutes to about 30 minutes.

16. The method of claims 13 to 15, wherein the hold temperature is 4° C.

17. The method of any one of the preceding claims, wherein index PCR is performed using about 10 to 24 cycles, each cycle comprising 15 to 50 minutes.

18. The method of claim 17, wherein the index PCR comprises a final extension comprising holding for about 5 minutes to 10 minutes.

19. The method of any one of the preceding claims, wherein the tagmented product is amplified with P7 and a primer comprising transposome-index-P5.

20. The method of any one of the preceding claims, wherein tagmentation comprises contacting the double stranded product with the transposome and a carrier gDNA.

21. The method of any one of the preceding claims, wherein the sequence that hybridizes to the untemplated cytosine nucleotides comprises 2-5 guanosines.

22. The method of claim 21, wherein the guanosines are riboguanosines.

23. The method of claim 22, wherein the sequence is rGrGrG.

24. A method for preparing a RNA sequence library, comprising:
mounting a tissue sample on a substrate comprising a plurality of capture oligonucleotides, wherein the capture oligonucleotides comprise a polyT sequence and library barcode information comprising a spatial barcode sequence (SBC);
capturing polyadenylated mRNA transcripts on the substrate with the capture oligonucleotides;
contacting the substrate with a first strand synthesis mix comprising a reverse transcriptase and a template switch oligo (TSO) under conditions to generate a first strand comprising a first cDNA complementary to the polyadenylated mRNA transcripts and a TSO complement hybridized to the 5′ end of the first cDNA;
eluting the polyadenylated mRNA transcripts from the substrate;
contacting the first strand with a blocker oligonucleotide and TSO to hybridize the blocker oligonucleotide and TSO to the first strand, wherein the blocker oligonucleotide and the TSO hybridize such that a gap is present between the blocker oligonucleotide and the TSO;
contacting the hybridized first strand with a non-strand displacing polymerase to gap fill the gap and generate a second cDNA;
removing the blocker oligonucleotide to form a blocker-free first strand;
contacting the blocker-free first strand with a transposome under conditions to tagment the blocker-free first strand to form a tagmented product comprising a unique molecular identifier (UMI) and a PCR adapter;
contacting the tagmented product with an extension mix to generate a second strand complementary to the first strand, the second strand comprising second strand barcode information having a spatial barcode sequence complement (SBC′) complementary to the spatial barcode sequence, the second cDNA, the unique molecular identifier, and the PCR adapter;
eluting the second strand; and
amplifying the second strand using index PCR to generate the library.

25. The method of claim 24, wherein the blocker oligonucleotide is a 3′-blocked SBS12′-PolyA oligonucleotide.

26. A method for preparing a RNA sequence library, comprising:
mounting a tissue sample on a substrate comprising a plurality of capture oligonucleotides, wherein the capture oligonucleotides comprise a polyT sequence and a barcode comprising a spatial barcode sequence (SBC);
capturing polyadenylated mRNA transcripts on the substrate with the capture oligonucleotides;
contacting the substrate with a first strand synthesis mix comprising a reverse transcriptase under conditions to generate a first strand comprising a first cDNA complementary to the polyadenylated mRNA transcripts;
eluting the polyadenylated mRNA transcripts from the substrate;
contacting the first strand with a second strand synthesis mix comprising a random primer to generate a second strand comprising a second cDNA and a unique molecular identifier (UMI);
eluting the second strand;
amplifying the second strand to produce a double stranded product;
contacting the double stranded product with a transposome under conditions to form a tagmented product; and
generating the library by amplifying the tagmented product in a first index PCR to determine the SBC and amplifying the tagmented product in a second index PCR to determine the UMI.

27. A method for preparing an RNA sequence library, comprising:
mounting a tissue sample on a substrate comprising a plurality of capture oligonucleotides, wherein the capture oligonucleotides comprise one or more gene-specific capture sequences and library barcode information comprising a spatial barcode sequence (SBC);
capturing mRNA transcripts on the substrate with the capture oligonucleotides;
contacting the substrate with a first strand synthesis mix comprising a reverse transcriptase and a template switch oligonucleotide (TSO) under conditions to generate a first strand comprising a first cDNA complementary to the mRNA transcripts, and a TSO complement hybridized to the 5′ end of the first cDNA, wherein the reverse transcriptase incorporates untemplated cytosine nucleotides at the 5′ end of the first cDNA and the TSO comprises a sequence that hybridizes to the untemplated cytosine nucleotides and the reverse transcriptase extends to generate the TSO complement, as a compliment of the TSO, at the 5′ end of the first cDNA;
eluting the mRNA transcripts from the substrate;
contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial barcode sequence complement (SBC′) that is complementary to the spatial barcode sequence (SBC);
eluting the second strand;
contacting the second strand with an extension mix comprising an extension primer and extending the extension primer using the second strand as a template to generate a double-stranded product while maintaining a single-stranded 3′ region containing the library barcode information, wherein the extension primer hybridizes to a region of the second strand that does not contain the second strand barcode information;
contacting the double stranded product with a transposome under conditions to tagment the double stranded product to form a tagmented product comprising a unique molecular identifier (UMI) and PCR adapter; and
amplifying the tagmented product using index PCR to generate the library.

28. The method of claim 27, wherein the second strand is neutralized after elution and prior to contacting with the extension mix.

29. The method of claim 27 or 28, wherein contacting the first strand with the second strand synthesis mix comprises incubating the first strand with the second strand synthesis mix for an incubation time of less than 2 hours.

30. The method of claim 29, wherein the incubation time is about 10 minutes to about 60 minutes.

31. The method of claim 30, wherein the incubation time is about 15 minutes to about 30 minutes.

32. The method of any one of claims 27 to 31, wherein the transposome is A14 transposome or B15 transposome.

33. The method of any one of claims 27 to 32, wherein the transposome is a transposome modified bead.

34. The method of any one of claims 27 to 33, comprising purifying the double stranded product before tagmentation.

35. The method of any one of claims 27 to 34, comprising purifying the double stranded product after index PCR.

36. The method of any one of claims 27 to 35, wherein contacting the second strand with the extension mix comprising incubating the second strand with the extension mix at a first temperature and first time, at a second temperature higher than the first temperature for a second time, and holding at a hold temperature.

37. The method of claim 36, wherein the first temperature is about 25° C. to about 37° C., the first time is about 10 minutes to about 30 minutes.

38. The method of claim 36 or 37, wherein the second temperature is about 60° C. to about 65° C., the second time is about 10 minutes to about 30 minutes.

39. The method of claims 36 to 38, wherein the hold temperature is 4° C.

40. The method of any one of claims 36 to 39, wherein index PCR is performed using about 10 to 24 cycles, each cycle comprising 15 to 50 minutes.

41. The method of claim 40, wherein the index PCR comprises a final extension comprising holding for about 5 minutes to 10 minutes.

42. The method of any one of claims 36 to 41, wherein the tagmented product is amplified with P7 and a primer comprising transposome-index-P5.

43. The method of any one of claims 36 to 42, wherein tagmentation comprises contacting the double stranded product with the transposome and a carrier gDNA.

44. The method of any one of claims 36 to 43, wherein the sequence that hybridizes to the untemplated cytosine nucleotides comprises 2-5 guanosines.

45. The method of claim 44, wherein the guanosines are riboguanosines.

46. The method of claim 45, wherein the sequence is rGrGrG.

47. A method for preparing a RNA sequence library, comprising:
mounting a tissue sample on a substrate comprising a plurality of capture oligonucleotides, wherein the capture oligonucleotides comprise one or more gene-specific capture sequences and library barcode information comprising a spatial barcode sequence (SBC);
capturing mRNA transcripts on the substrate with the capture oligonucleotides;
contacting the substrate with a first strand synthesis mix comprising a reverse transcriptase and a template switch oligo (TSO) under conditions to generate a first strand comprising a first cDNA complementary to the mRNA transcripts and a TSO complement hybridized to the 5′ end of the first cDNA;
eluting the mRNA transcripts from the substrate;
contacting the first strand with a blocker oligonucleotide and TSO to hybridize the blocker oligonucleotide and TSO to the first strand, wherein the blocker oligonucleotide and the TSO hybridize such that a gap is present between the blocker oligonucleotide and the TSO;
contacting the hybridized first strand with a non-strand displacing polymerase to gap fill the gap and generate a second cDNA;
removing the blocker oligonucleotide to form a blocker-free first strand;
contacting the blocker-free first strand with a transposome under conditions to tagment the blocker-free first strand to form a tagmented product comprising a unique molecular identifier (UMI) and a PCR adapter;
contacting the tagmented product with an extension mix to generate a second strand complementary to the first strand, the second strand comprising second strand barcode information having a spatial barcode sequence complement (SBC′) complementary to the spatial barcode sequence, the second cDNA, the unique molecular identifier, and the PCR adapter;
eluting the second strand; and
amplifying the second strand using index PCR to generate the library.

48. A method for preparing a RNA sequence library, comprising:
mounting a tissue sample on a substrate comprising a plurality of capture oligonucleotides, wherein the capture oligonucleotides comprise one or more gene-specific capture sequences and a barcode comprising a spatial barcode sequence (SBC);
capturing mRNA transcripts on the substrate with the capture oligonucleotides;
contacting the substrate with a first strand synthesis mix comprising a reverse transcriptase under conditions to generate a first strand comprising a first cDNA complementary to the mRNA transcripts;
eluting the mRNA transcripts from the substrate;
contacting the first strand with a second strand synthesis mix comprising a random primer and extending the random primer to generate a second strand comprising a second cDNA and a unique molecular identifier (UMI);
eluting the second strand;
amplifying the second strand to produce a double stranded product;
contacting the double stranded product with a transposome under conditions to form a tagmented product; and
generating the library by amplifying the tagmented product in a first index PCR to determine the SBC and amplifying the tagmented product in a second index PCR to determine the UMI.

49. A method for preparing a spatially barcoded RNA library from a tissue sample comprising,
a) contacting the tissue sample with a plurality of capture oligonucleotides immobilized on a solid substrate and capable of hybridizing with RNA in the tissue sample, wherein the capture oligonucleotides comprise a capture nucleotide sequence, a spatial barcode sequence (SBC), and adapter sequences, wherein RNA transcripts are captured by the capture nucleotide sequence of the plurality of capture oligonucleotides;
b) contacting the RNA transcripts with a first strand synthesis mix comprising a reverse transcriptase (RT) and a template switch oligonucleotide (TSO) encoding a first adapter sequence under conditions to generate a first strand cDNA comprising
a first strand cDNA complementary to the RNA transcripts and a TSO appended to a 3′ end of the first cDNA, wherein the reverse transcriptase incorporates untemplated cytosine nucleotides at the 3′ end of the first strand cDNA and the TSO comprising a first adapter sequence is hybridized to the 3′ end of the first cDNA and the reverse transcriptase extends to generate a TSO complement; wherein the contacting generates a mixture of template switched molecules and non-template switched molecules, wherein the non-template switched molecules lack the complement to the first adapter sequence;
c) contacting the mixture with a plurality of oligo ligation blockers comprising the first adapter sequence and capable of hybridizing with the template switched molecule 3′ end comprising the complement to the first adapter sequence;
d) carrying out a single-stranded ligation step comprising hybridizing a splint adapter to the non-template-switched molecules, wherein the splint adapter comprises i) a single-stranded splint sequence comprising a random base sequence (NX) and ii) a double-stranded first adapter sequence comprising hybridized first adapter and complement to the first adapter sequences, wherein the complement to the first adapter sequence 5′ end contains a phosphate group for ligation to the captured non-template switched molecules 3′OH end, and ligating the 5′ end of the splint adapter complement to the 3′OH end of the non-template switched molecules; and
e) removing the ligation blockers and the splint sequence and first adapter from the ligated molecules.

50. The method of claim 49 comprising contacting the mixture of template switched molecules and non-template switched molecules with an exonuclease.

51. The method of claim 50, wherein the exonuclease is DNA exonuclease I or RNAse H.

52. The method of any one of claims 49-51, wherein the NX sequence has a blocking group at the NX sequence 5′ end.

53. The method of any one of claims 49-52, wherein the hybridized first adapter and complement to the first adapter sequences comprise blocking groups at ends of both the first adapter and complement to the first adapter sequences furthest from the splint sequence.

54. The method of any one of claims 50-53 further comprising after the exonuclease, contacting the mixture with an alkaline solution.

55. The method of any one of claims 49-54 further comprising, after the removing step, amplifying the template switched and non template switched molecules by contacting the mixture with a second strand synthesis mix comprising a single first adapter primer and extending the first adapter primer using the first strand cDNA or complement thereof as a template to generate a second strand cDNA complementary to the first strand or complement thereof, the second strand cDNA comprising a second cDNA complementary to the first strand cDNA, and second strand barcode information comprising a spatial barcode sequence complement (SBC′) that is complementary to the spatial barcode sequence (SBC) in the capture oligonucleotide.

56. The method of claim 55, wherein the first adapter primer is a full length primer or partial primer.

57. The method of any one of claims 49-56 further comprising eluting the amplified first strand and/or second strand cDNA molecules from the substrate and generating a spatially barcoded RNA library from the eluted molecules using a library prep kit.

58. The method of any one of claims 49-57, wherein the ligated molecules of step (d) further comprise a cleavage sequence.

59. The method of claim 58, wherein removing the ligation blockers, splint sequence and first adapter from the ligated molecules is carried out off the substrate.

60. The method of claim 57 or 58, wherein amplifying the template switched and non template switched molecules, eluting the second strand cDNA and generating a spatially barcoded library are performed in solution.

61. The method of any one of claims 49-60, wherein the amplifying step is carried out on the substrate.

62. The method of claim 61, wherein the amplified first strand and/or second strand further comprise a cleavage sequence.

63. The method of claim 61 or 62, wherein eluting the second strand cDNA and generating a spatially barcoded library are performed in solution.

64. A method for preparing a spatially barcoded RNA library from a tissue sample comprising,
a) contacting the tissue sample with a plurality of capture oligonucleotides immobilized on a solid substrate and capable of hybridizing with RNA in the tissue sample, wherein the capture oligonucleotides comprise a capture nucleotide sequence, a spatial barcode sequence (SBC), and adapter sequences, wherein RNA transcripts are captured by the capture nucleotide sequence of the plurality of capture oligonucleotides;
b) contacting the RNA transcripts with a first strand synthesis mix comprising a reverse transcriptase (RT) and a template switch oligonucleotide (TSO) encoding a first adapter sequence under conditions to generate a first strand cDNA comprising
a first strand cDNA complementary to the RNA transcripts and a TSO hybridized to a 3′ end of the first strand cDNA, wherein the reverse transcriptase incorporates untemplated cytosine nucleotides at the 3′ end of the first strand cDNA and the TSO comprising a first adapter sequence is appended to the 3′ end of the first strand cDNA and the reverse transcriptase extends to generate a TSO complement; wherein the contacting generates a mixture of template switched molecules and non-template switched molecules, wherein the non-template switched molecules lack the complement to the first adapter sequence;
c) contacting the mixture with a plurality of oligo ligation blockers comprising the first adapter sequence and capable of hybridizing with the template switched molecule 3′ end comprising the complement to the first adapter sequence and a plurality of complementary oligo blockers comprising nucleotide sequences complementary to all or part of the capture nucleotide sequence and a fixed sequence in the capture oligonucleotide to generate a double-stranded 3′ terminus of the capture oligonucleotide;
d) carrying out a single-stranded ligation step comprising hybridizing a splint adapter to the non-template-switched molecules, wherein the splint adapter comprises i) a single-stranded splint sequence comprising a random base sequence (NX) and ii) a double-stranded partial first adapter sequence comprising hybridized first adapter and complement to the first adapter sequences, wherein the complement to the first adapter sequence 5′ end contains a phosphate group for ligation to the captured non-template switched molecules 3′OH end, and ligating the 5′ end of the splint adapter complement to the 3′ OH end of the non-template switched molecules; and
e) removing the ligation blockers and the splint strand of the adapter.

65. The method of claim 64 comprising contacting the mixture of template switched molecules and non-template switched molecules with an exonuclease.

66. The method of claim 65, wherein the exonuclease is DNA exonuclease I or RNAse H.

67. The method of any one of claims 64-66, wherein the NX sequence has a blocking group at the NX sequence 5′ end.

68. The method of any one of claims 64-67, wherein the hybridized first adapter and complement to the first adapter sequences comprise blocking groups at ends of both the first adapter and complement to the first adapter sequences furthest from the splint sequence.

69. The method of any one of claims 65-68 further comprising after the exonuclease, contacting the mixture with an alkaline solution.

70. The method of any one of claims 64-69 further comprising, after the removing step, amplifying the template switched and non template switched molecules by contacting the mixture with a second strand synthesis mix comprising a single first adapter primer and extending the first adapter primer using the first strand cDNA as a template to generate a second strand cDNA complementary to the first strand, the second strand cDNA comprising a second cDNA complementary to the first strand cDNA, and second strand barcode information comprising a spatial barcode sequence complement (SBC′) that is complementary to the spatial barcode sequence (SBC) in the capture oligonucleotide.

71. The method of claim 70, wherein the first adapter primer is a full length primer or partial primer.

72. The method of any one of claims 49-71 further comprising eluting the amplified first strand and/or second strand cDNA molecules from the substrate and generating a spatially barcoded RNA library from the eluted molecules using a library prep kit.

73. The method of any one of claims 65-72, wherein the ligated molecules of step (d) further comprise a cleavage sequence.

74. The method of claim 73, wherein removing the ligation blockers, splint sequence and first adapter from the ligated molecules is carried out off the substrate.

75. The method of claim 73 or 74, wherein amplifying the template switched and non template switched molecules, eluting the second strand cDNA and generating a spatially barcoded library are performed in solution.

76. The method of any one of claims 49-75, wherein the amplifying step is carried out on the substrate.

77. The method of claim 76, wherein the amplified first strand and/or second strand further comprise a cleavage sequence.

78. The method of claim 76 or 77, wherein eluting the second strand cDNA and generating a spatially barcoded library are performed in solution.

79. The method of any one of claims 49-78 optionally comprising mounting the tissue sample on a substrate comprising the plurality of capture oligonucleotides prior to contacting the tissue with the plurality of capture oligonucleotides.

80. The method of any one of claims 49-79, wherein the capture nucleotide sequence is a poly-T sequence, a poly-A sequence, a gene-specific capture sequence, or a universal capture sequence.

81. The method of claim 80, wherein the universal capture sequence is a random nucleotide sequence or a non-self complementary semi-random sequence.

82. The method of any one of claims 49-81, wherein the capture oligonucleotide comprises; a 5′ clustering sequence, a randomized spatial barcode (SBC), a full-length second adapter sequence (2 FL), a molecular identifier (MI), a fixed sequence (FS), and a poly T capture sequence with a 3′ VN terminus (polyTVN).

83. The method of claim 82, wherein the first adapter sequence is a read 1 (Rd1) sequence, and the second adapter is a read 2 (Rd2) sequence.

84. The method of any one of claims 49-83, wherein the first adapter is a partial adapter sequence.

85. The method of claim 84, wherein the molecular identifier is a unique molecular identifier, an endogenous molecular identifier, an exogenous molecular identifier, or a virtual molecular identifier.

86. The method of any one of claims 49-85, wherein the ligation step comprises enzymatic ligation of the splint adapter to the non-template switched molecule.

87. The method of claim 86, wherein the enzymatic ligation is by T4 ligase, other DNA ligase, or thermostable 5′ App DNA/RNA ligase-mediated ligation with a synthesized pre-adenylated single-stranded oligo adapter.

88. The method of any one of claims 49-87, wherein the ligation step comprises chemical ligation of the splint adapter to the non-template switched molecule.

89. The method of claim 88, wherein the chemical ligation is carried out using click-chemistry-mediated ligation wherein 3′ azido termini are joined to synthesized 5′ alkyne single-stranded oligos or 1-ethyl-3-(3-dimethylaminopropyl) carbodiimide (EDC)-mediated ligation wherein cDNAs with 3′ phosphate groups are ligated to splinted adapters with 5′ hydroxyl termini.

90. The method of claim 89, wherein 3′ azido-ddNTPs or 3′ Phos-dATPs are incorporated onto the Rd1 adapter during first strand cDNA synthesis.

91. The method of any one of claims 49-90, wherein the splint adapter random sequence comprises between 6 and 10 nucleotides.

92. The method of any one of claims 49-91, wherein the splint adapter random sequence comprises 7 nucleotides.

93. The method of any one of claims 49-92, wherein the splint adapter comprises blocking groups at both 3′ ends and the 5′ end of the splint sequence and a ligation blocking group at the 5′ end of the adapter strand that is complementary to the splinted strand.

94. The method of claim 93, wherein the ligation blocker group is a phosphate.

95. The method of any one of claims 49-94, wherein the ligation blocker and splint adapter are removed by alkaline treatment.

96. The method of claim 95, wherein the alkaline treatment comprises either 0.08 M KOH or 0.1 N NaON for five minutes at room temperature.

97. The method of any one of claims 49-96, wherein the 5′ clustering sequence comprises a P7 sequence.

98. The method of any one of claims 49-97, wherein the capture oligonucleotide further comprises a randomer, a semi-random sequence, or a target-specific probe.

99. The method of any one of claims 49-98, wherein the sequence that hybridizes to the untemplated cytosine nucleotides comprises 2-5 guanosines.

100. The method of claim 99, wherein the guanosines are riboguanosines, modified nucleic acids or locked nucleic acids (LNA).

101. The method of claim 100, wherein the sequence is rGrGrG.

102. The method of any one of claims 49-101, wherein the polyT sequence is between 20-30 nucleotides.

103. The method of any one of claims 49-102, wherein the SBC is a randomer.

104. The method of any one of claims 49-103, wherein the SBC is between 20 and 30 nucleotides.

105. The method of any one of claims 49-104, wherein the capture oligonucleotide comprises at least 10 deoxythymidine residues.

106. The method of claim 105, wherein the capture oligonucleotide comprises a plurality of different target-specific RNA capture probe sequences.

107. The method of claim 78, wherein the target-specific probes comprise at least 10 nucleotides complementary to a nucleotide sequence of a target RNA.

108. The method of any one of claims 49-107, wherein the capture oligonucleotide is between 8 to 80 nucleotides.

109. The method of any one of claims 49-108, further comprising, prior to the step of capturing RNA from the tissue sample, the step of performing end repair of the RNA with polynucleotide kinase.

110. The method of any one of claims 49-108, further comprising, prior to the step of capturing RNA from the tissue sample, the step of performing in situ polyadenylation with polyadenylate polymerase.

111. The method of any one of claims 49-108, further comprising, prior to the step of capturing RNA from the tissue sample, the steps of performing end repair of the RNA with polynucleotide kinase followed by performing in situ polyadenylation with polyadenylate polymerase.

112. The method of any one of claims 49-111, wherein the RNA comprises ribosomal RNA (rRNA), messenger RNA (mRNA), non-coding RNA (ncRNA), small nuclear RNA (snRNA), small nucleolar RNA (snoRNA), and/or microRNA (miRNA).

113. The method of any one of claims 49-112, wherein the tissue sample is formalin-fixed paraffin embedded (FFPE) tissue or fresh frozen (FF) tissue.

114. The method of any one of claims 49-113, wherein removing the RNA is carried out by melting the RNA or digestion with an RNase.

115. The method of any one of claims 49-114, wherein the tissue sample is permeabilized prior to contacting the tissue sample with a plurality of capture oligonucleotides.

116. The method of any one of claims 49-115, wherein the tissue sample is treated with one or more blocking reagents prior to contacting the tissue sample with a plurality of capture oligonucleotides.

117. The method of any one of claims 49-116, wherein the tissue sample is permeabilized and treated with one or more blocking reagents prior to contacting the tissue sample with a plurality of capture oligonucleotides.

118. The method of any one of claims 49-117, wherein the methods comprise polyadenylating the RNA in the sample.

119. The method of claim 118, wherein the RNA is polyadenylated using a poly(A) polymerase.

120. The method of claim 118, wherein the RNA is polyadenylated using chemical ligation or enzymatic ligation.

121. The method of any one of claims 49-120, wherein the substrate is a bead, a bead array, a spotted array, a substrate comprising a plurality of wells, a flow cell, clustered particles arranged on a surface of a chip, a film, or a plate.

122. The method of claim 121, wherein the substrate comprises a plurality of nanowells or microwells.

123. The method of any one of claims 49-122, wherein the RNA library is an mRNA library.

124. The method of any one of claims 49-123, further comprising indexing and sequencing the second strand cDNA comprising,
performing PCR on the second strand cDNA to yield a PCR template representative of one or more RNA transcripts in the tissue sample;
eluting the PCR template; and
carrying out an indexing PCR to generate a double stranded PCR product comprising the first strand PCR product and a second strand complementary to the first strand PCR product.

125. The method of claim 124, further comprising sequencing the PCR product and determining the location of the RNA transcript in the tissue based on the spatial barcode.

126. The method of claim 124 or 125, wherein the double stranded PCR product comprises a second clustering sequence on the second strand complementary to the first strand PCR product and, optionally, an index sequence.

127. The method of claim 124 or 125, wherein the double stranded PCR product is further processed by tagmentation to generate a spatial transcriptomics library.

128. The method of claim 127, wherein the tagmentation comprises on substrate tagmentation.

129. The method of claim 127 or 128, wherein tagmentation comprises contacting the double stranded product with the transposome and a carrier gDNA.

130. The method of any one of claims 49-129, further comprising determining spatial locations of the spatial barcodes of the plurality of capture oligonucleotide molecules prior to the step of contacting the tissue with the substrate.

131. The method of claim 130, further comprising sequencing at least a portion of the spatially barcoded first strand cDNA or copies thereof to determine the spatial barcode sequence for each molecule.

132. The method of claim 131, wherein the spatially barcoded first strand cDNA is sequenced in situ.

133. The method of claim 131 or 132, further comprising determining the spatial location of one or more of the spatially barcoded first strand cDNA or copies thereof by correlating the spatial barcode sequences of the spatially barcoded first strand cDNA or copies thereof with the spatial locations of the capture oligonucleotide molecules on the substrate containing corresponding spatial barcode sequences.

134. The method of claim 132, further comprising recovering the spatially barcoded first strand cDNA and amplifying the first strand cDNA to generate cDNA libraries.

135. The method of claim 134, wherein the spatially barcoded first strand cDNA is recovered by contacting the spatially barcoded first strand cDNAs on the substrate with a DNA polymerase and one or more primers to generate spatially barcoded second strand cDNAs complementary to the spatially barcoded first strand cDNAs and removing the spatially barcoded second strand cDNAs from the substrate.

136. The method of claim 135, wherein the one or more primers each comprise a random priming sequence.

137. The method of claim 136, wherein the random priming sequences comprises nine random nucleotides.

138. The method of claim 136 or 137, wherein the spatially barcoded second strand cDNAs each comprise a unique molecular identifier (UMI), wherein the UMI comprises an intrinsic sequence and an extrinsic sequence, wherein the extrinsic sequence is a sequence complementary to the random priming sequence used to generate the second strand cDNA, and wherein the intrinsic sequence is a sequence complementary to the first strand cDNA template sequence used to generate the second strand cDNA.

139. The method of claim 135, wherein the one or more primers each comprise a molecular identifier barcode.

140. The method of claim 135, wherein the one or more primers each comprise a UMI barcode.

141. The method of any one of claims 135-140, wherein the spatially barcoded second strand cDNAs are removed from the substrate by chemical or physical dehybridization.

142. The method of claim 135, wherein the capture oligonucleotide comprises an anchor sequence comprising a cleavage site that anchors the capture oligonucleotide to the substrate, and hybrids of the spatially barcoded first and second strand cDNAs are removed from the substrate by enzymatic cleavage at the cleavage site.

143. The method of claim 142, wherein the cleavage site is a binding site for a restriction endonuclease.

144. The method of any one of claims 134-143, further comprising sequencing at least a portion of the cDNA libraries to determine the spatial barcode sequence for each molecule.

145. The method of claim 144, further comprising determining the spatial location of one or more cDNA molecules by correlating the spatial barcode sequences of the one or more cDNA molecules with the spatial locations of the surface oligonucleotide molecules on the substrate containing corresponding spatial barcode sequences.

146. The method of any one of claims 49-145, wherein RNA expression in a single cell within the tissue is determined.

147. The method of any one of claims 49-146, wherein RNA expression in a subcellular component within a single cell is determined.

148. The method of claim 147, wherein the subcellular component is a nucleus, mitochondria, ribosomes or cytoplasm.

149. The method of any one of claims 49-148, wherein the substrate or surface of the substrate comprises a material selected from glass, silicon, poly-L-lysine coated materials, nitrocellulose, polystyrene, cyclic olefin copolymers (COCs), cyclic olefin polymers (COPs), polyacrylamide, polypropylene, polyethylene, or polycarbonate.

150. A kit comprising
a) a solid substrate comprising capture oligonucleotides immobilized on the solid substrate, wherein the capture oligonucleotides comprise a capture nucleotide sequence, a spatial barcode sequence (SBC), and adapter sequences;
b) a reverse transcriptase (RT) and a template switch oligonucleotide (TSO) encoding a first adapter sequence); and
c) a splint adapter, wherein the splint adapter comprises
i) a single-stranded splint sequence comprising a random base sequence (NX) having a blocking group at the NX sequence 5′ end; and
ii) a double-stranded first adapter sequence comprising hybridized first adapter sequence and complementary to first adapter sequences, optionally wherein the hybridized first adapter sequence and complementary to first adapter sequences comprise blocking groups at the 5′ end of the first adapter and the 3′ end of the complement to first adapter sequences.

151. A method of preparing an immobilized library of target nucleic acids of a biological sample, comprising:
(a) providing a surface comprising a plurality of capture oligonucleotides immobilized thereon, wherein one or more of the plurality of capture oligonucleotides comprises, from 5′ to 3′:
(i) a first clustering primer sequence;
(ii) a spatial barcode (SBC) sequence;
(iii) a first sequencing primer sequence; and
(iv) a capture nucleotide sequence;
(b) contacting the biological sample with the surface, the contacting resulting in hybridization of the target nucleic acids of the biological sample to the capture nucleotide sequence of the plurality of capture oligonucleotides to form hybridized capture oligonucleotides;
(c) extending the capture nucleotide sequence of the hybridized capture oligonucleotides to form first complementary strands of the target nucleic acids, wherein the extending is in the presence of an extension termination moiety, and wherein the extension termination moiety is an allyl-T or a deoxyuridine triphosphate (dUTP), thereby preparing the immobilized library of target nucleic acids.

152. The method of claim 151, further comprising:
(d) contacting the surface with an exonuclease;
(e) hybridizing a plurality of oligonucleotide primers to the first complementary strands, wherein each of the plurality of oligonucleotide primers comprises, from 5′ to 3′:
(i) an adapter nucleotide sequence; and
(ii) a random nucleotide sequence;
(f) extending the plurality of oligonucleotide primers, thereby generating one or more second complementary strands comprising the adapter nucleotide sequence at a terminus.

153. The method of claim 152, further comprising:
(g) removing the one or more second complementary strands from the surface and amplifying the one or more second complementary strands.

154. The method of claim 153, wherein step (g) is performed in the presence of an Exclusion Amplification (ExAmp) mix, wherein the ExAmp mix comprises a primer comprising the clustering primer sequence.

155. The method of claim 151, wherein one or more of the plurality of capture oligonucleotides is immobilized on the surface through a cleavage site.

156. The method of claim 155, wherein the cleavage site is an enzymatic cleavage site.

157. The method of claim 156, wherein the enzymatic cleavage site comprises a restriction enzyme site, a uracil, an 8-oxoguanine, or a combination thereof.

158. The method of claim 155, wherein the cleavage site is a chemical cleavage site.

159. The method of any one of claims 155-158, wherein the cleavage site is cleaved after step (c).

160. The method of claim 152, wherein one or more of the plurality of capture oligonucleotides is immobilized on the surface through a cleavage site.

161. The method of claim 160, wherein the cleavage site is an enzymatic cleavage site.

162. The method of claim 161, wherein the enzymatic cleavage site comprises a restriction enzyme site, a uracil, an 8-oxoguanine, or a combination thereof.

163. The method of claim 160, wherein the cleavage site is a chemical cleavage site.

164. The method of any one of claims 160-163, wherein the cleavage site is cleaved after step (f).

165. The method of any one of claims 151-164, wherein the extension termination moiety is a deoxyuridine triphosphate (dUTP) and the method further comprises contacting the surface with a uracil-DNA glycosylase (UDG).

166. The method of any one of claims 151-164, wherein the extension termination moiety is an allyl-T and wherein the method further comprises contacting the surface with a universal cleavage mix (UCM).

167. The method of any one of claims 152-164, wherein the extension termination moiety is a deoxyuridine triphosphate (dUTP) and the method further comprises contacting the surface with a uracil-DNA glycosylase (UDG).

168. The method of any one of claims 152-164, wherein the extension termination moiety is an allyl-T and wherein the method further comprises contacting the surface with a universal cleavage mix (UCM) prior to step (e).

169. A method of preparing an immobilized library of target nucleic acids of a biological sample, comprising:
(a) providing a surface comprising a plurality of capture oligonucleotides immobilized thereon, wherein one or more of the plurality of capture oligonucleotides comprises, from 5′ to 3′:
(i) a first clustering primer sequence;
(ii) a spatial barcode (SBC) sequence;
(iii) a first sequencing primer sequence; and
(iv) a capture nucleotide sequence;
(b) contacting the biological sample with the surface, the contacting resulting in hybridization of the target nucleic acids of the biological sample to the capture nucleotide sequence of the plurality of capture oligonucleotides to form hybridized capture oligonucleotides;
(c) extending the capture nucleotide sequence of the hybridized capture oligonucleotides to form first complementary strands of the target nucleic acids, wherein the extending is in the presence of an extension termination moiety, and wherein the extension termination moiety is a dideoxynucleoside triphosphate (ddNTP),
thereby preparing the immobilized library of target nucleic acids.

170. The method of claim 169, further comprising:
(d) contacting the surface with an exonuclease;
(e) hybridizing a plurality of oligonucleotide primers to the first complementary strands, wherein each of the plurality of oligonucleotide primers comprises, from 5′ to 3′:
(i) an adapter nucleotide sequence; and
(ii) a random nucleotide sequence;
(f) extending the plurality of oligonucleotide primers, thereby generating one or more second complementary strands comprising the adapter nucleotide sequence at a terminus.

171. The method of claim 170, further comprising:
(g) removing the one or more second complementary strands from the surface and amplifying the one or more second complementary strands.

172. The method of claim 171, wherein step (g) is performed in the presence of an Exclusion Amplification (ExAmp) mix, wherein the ExAmp mix comprises a primer comprising the first clustering primer sequence.

173. The method of claim 169, wherein one or more of the plurality of capture oligonucleotides is immobilized on the surface through a cleavage site.

174. The method of claim 173, wherein the cleavage site is an enzymatic cleavage site.

175. The method of claim 174, wherein the enzymatic cleavage site comprises a restriction enzyme site, a uracil, an 8-oxoguanine, or a combination thereof.

176. The method of claim 173, wherein the cleavage site is a chemical cleavage site.

177. The method of any one of claims 173-176, wherein the cleavage site is cleaved after step (c).

178. The method of claim 170, wherein one or more of the plurality of capture oligonucleotides is immobilized on the surface through a cleavage site.

179. The method of claim 178, wherein the cleavage site is an enzymatic cleavage site.

180. The method of claim 179, wherein the enzymatic cleavage site comprises a restriction enzyme site, a uracil, an 8-oxoguanine, or a combination thereof.

181. The method of claim 178, wherein the cleavage site is a chemical cleavage site.

182. The method of any one of claims 178-181, wherein the cleavage site is cleaved after step (f).

183. The method of any one of claims 169-182, wherein the ddNTP comprises a first click chemistry handle.

184. The method of claim 183, wherein the method further comprises, after step (c), contacting the surface with an adapter oligonucleotide comprising a second click chemistry handle capable of crosslinking to the first click chemistry handle, thereby ligating the adapter oligonucleotide to the first complementary strands.

185. The method of claim 184, wherein the adapter oligonucleotide further comprises a second sequencing primer sequence.

186. The method of claim 184 or claim 185, wherein the first click chemistry handle is an azide, a tetrazine, a strained alkene, or an alkyne.

187. The method of any one of claims 184-186, wherein the second click chemistry handle is an azide, a tetrazine, a strained alkene, or an alkyne.

188. A method of preparing an immobilized library of target nucleic acids of a biological sample, comprising:
(a) providing a surface comprising a plurality of capture oligonucleotides immobilized thereon, wherein one or more of the plurality of capture oligonucleotides comprises, from 5′ to 3′:
(i) a first clustering primer sequence;
(ii) a spatial barcode (SBC) sequence;
(iii) a first sequencing primer sequence; and
(iv) a capture nucleotide sequence;
(b) contacting the biological sample with the surface, the contacting resulting in hybridization of the target nucleic acids of the biological sample to the capture nucleotide sequence of the plurality of capture oligonucleotides to form hybridized capture oligonucleotides;
(c) extending the capture nucleotide sequence of the hybridized capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate, thereby preparing the immobilized library of target nucleic acids.

189. The method of claim 188, further comprising:
(d) contacting the surface with an exonuclease; and
(e) contacting the surface with a ligase enzyme, thereby ligating an adapter oligonucleotide to the first complementary strands, wherein the adapter oligonucleotide comprises, from 5′ to 3′:
(i) an adapter nucleotide sequence; and
(ii) a random nucleotide sequence,
and wherein the adapter oligonucleotide further comprises a second oligonucleotide that is hybridized to the adapter nucleotide sequence.

190. The method of claim 189, wherein the adapter nucleotide sequence comprises a second sequencing primer sequence.

191. The method of claim 189, wherein the ligating occurs through a splinted ligation of the adapter oligonucleotide to the first complementary strands.

192. The method of any one of claims 189-191, wherein the ligase enzyme is a T4 DNA ligase.

193. The method of any one of claims 189-192, further comprising:
(f) extending the adapter oligonucleotide, thereby generating one or more second complementary strands.

194. The method of claim 188, further comprising:
(d) contacting the surface with an exonuclease; and
(e) contacting the surface with a ligase enzyme, thereby ligating an adapter oligonucleotide to the first complementary strands, wherein the adapter oligonucleotide comprises, from 5′ to 3′:
(i) a random nucleotide sequence; and
(ii) an adapter nucleotide sequence.

195. The method of claim 194, wherein the adapter nucleotide sequence comprises a second sequencing primer sequence.

196. The method of claim 194 or claim 195, wherein the ligating occurs through a single-stranded DNA ligation of the adapter oligonucleotide to the first complementary strands.

197. The method of any one of claims 194-196, wherein the ligase enzyme is a DNA/RNA ligase.

198. The method of any one of claims 194-197, further comprising:
(f) extending the adapter oligonucleotide, thereby generating one or more second complementary strands.

199. The method of claim 193 or claim 198, further comprising:
(g) removing the one or more second complementary strands from the surface and amplifying the one or more second complementary strands.

200. The method of claim 199, wherein step (g) is performed in the presence of an Exclusion Amplification (ExAmp) mix.

201. The method of claim 188, wherein one or more of the plurality of capture oligonucleotides is immobilized on the surface through a cleavage site.

202. The method of claim 201, wherein the cleavage site is an enzymatic cleavage site.

203. The method of claim 202, wherein the enzymatic cleavage site comprises a restriction enzyme site, a uracil, an 8-oxoguanine, or a combination thereof.

204. The method of claim 201, wherein the cleavage site is a chemical cleavage site.

205. The method of any one of claims 201-204, wherein the cleavage site is cleaved after step (c).

206. The method of claim 189 or claim 194, wherein one or more of the plurality of capture oligonucleotides is immobilized on the surface through a cleavage site.

207. The method of claim 206, wherein the cleavage site is an enzymatic cleavage site.

208. The method of claim 207, wherein the enzymatic cleavage site comprises a restriction enzyme site, a uracil, an 8-oxoguanine, or a combination thereof.

209. The method of claim 206, wherein the cleavage site is a chemical cleavage site.

210. The method of any one of claims 206-209, wherein the cleavage site is cleaved after step (e).

211. A method of preparing an immobilized library of target nucleic acids of a biological sample, comprising:
(a) providing a surface comprising a plurality of capture oligonucleotides immobilized thereon, wherein one or more of the plurality of capture oligonucleotides comprises, from 5′ to 3′:
(i) a first clustering primer sequence;
(ii) a spatial barcode (SBC) sequence;
(iii) a first sequencing primer sequence; and
(iv) a capture nucleotide sequence;
(b) contacting the biological sample with the surface, the contacting resulting in hybridization of the target nucleic acids of the biological sample to the capture nucleotide sequence of the plurality of capture oligonucleotides to form hybridized capture oligonucleotides;
(c) extending the capture nucleotide sequence of the hybridized capture oligonucleotides to form first complementary strands of the target nucleic acids, wherein the extending is in the presence of an extension termination moiety, and wherein the extension termination moiety is a deoxynucleoside triphosphate (dNTP) comprising a 3′ phosphate or a dideoxynucleoside triphosphate (ddNTP) comprising a first click chemistry handle,
thereby preparing the immobilized library of target nucleic acids.

212. The method of claim 211, wherein the extension termination moiety is the deoxynucleoside triphosphate (dNTP) comprising a 3′ phosphate.

213. The method of claim 211 or claim 212, further comprising:
(d) chemically ligating an adapter oligonucleotide to the first complementary strands through a crosslinking group, wherein the adapter oligonucleotide comprises, from 5′ to 3′:
(i) an adapter nucleotide sequence; and
(ii) a random nucleotide sequence,
and wherein the adapter oligonucleotide further comprises a second oligonucleotide that is hybridized to the adapter nucleotide sequence.

214. The method of claim 213, wherein the adapter nucleotide sequence comprises a second sequencing primer sequence.

215. The method of claim 213, wherein the crosslinking group is a carboxyl-to-amine reactive group, a BCN-azide reactive group, a DBCO-azide reactive group, a Tetrazine-TCO reactive group, or a combination thereof.

216. The method of claim 211, wherein the extension termination moiety is the dideoxynucleoside triphosphate (ddNTP) comprising the first click chemistry handle.

217. The method of claim 211 or claim 216, further comprising:
(d) ligating an adapter oligonucleotide to the first complementary strands through click chemistry, wherein the adapter oligonucleotide comprises, from 5′ to 3′:
(i) an adapter nucleotide sequence; and
(ii) a random nucleotide sequence,
and wherein the adapter oligonucleotide further comprises a second oligonucleotide that is hybridized to the sequencing primer sequence, wherein the second oligonucleotide comprises a second click chemistry handle.

218. The method of claim 217, wherein the adapter nucleotide sequence comprises a second sequencing primer sequence.

219. The method of any one of claim 211, 215, or 217, wherein the first click chemistry handle is an azide, a tetrazine, a strained alkene, or an alkyne.

220. The method of any one of claims 217-219, wherein the second click chemistry handle is an azide, a tetrazine, a strained alkene, or an alkyne.

221. The method of any one of claims 213-220, further comprising:
(e) extending the adapter oligonucleotide, thereby generating one or more second complementary strands.

222. The method of claim 221, further comprising:
(f) removing the one or more second complementary strands from the surface and amplifying the one or more second complementary strands.

223. The method of claim 222, wherein step (f) is performed in the presence of an Exclusion Amplification (ExAmp) mix.

224. The method of claim 211, wherein one or more of the plurality of capture oligonucleotides is immobilized on the surface through a cleavage site.

225. The method of claim 224, wherein the cleavage site is an enzymatic cleavage site.

226. The method of claim 225, wherein the enzymatic cleavage site comprises a restriction enzyme site, a uracil, an 8-oxoguanine, or a combination thereof.

227. The method of claim 224, wherein the cleavage site is a chemical cleavage site.

228. The method of any one of claims 224-227, wherein the cleavage site is cleaved after step (c).

229. The method of claim 213 or claim 217, wherein one or more of the plurality of capture oligonucleotides is immobilized on the surface through a cleavage site.

230. The method of claim 229, wherein the cleavage site is an enzymatic cleavage site.

231. The method of claim 230, wherein the enzymatic cleavage site comprises a restriction enzyme site, a uracil, an 8-oxoguanine, or a combination thereof.

232. The method of claim 229, wherein the cleavage site is a chemical cleavage site.

233. The method of any one of claims 229-232, wherein the cleavage site is cleaved after step (d).

234. The method of any one of claims 151-233, further comprising removing the target nucleic acids from the surface after step (c).

235. The method of any one of claims 151-234, further comprising removing the biological sample from the surface after step (d).

236. The method of any one of claims 151-235, wherein each of the plurality of capture oligonucleotides comprises the same capture nucleotide sequence.

237. The method of any one of claims 151-235, wherein the plurality of capture oligonucleotides comprises multiple, different capture nucleotide sequences.

238. The method of claim 237, wherein the multiple, different capture nucleotide sequences comprise one or more gene-specific capture sequences, one or more universal capture sequences, or a combination thereof.

239. The method of any one of claims 151-237, wherein the capture nucleotide sequence is a poly-T sequence, a poly-A sequence, a gene-specific capture sequence, or a universal capture sequence.

240. The method of claim 238 or claim 239, wherein the universal capture sequence is a random nucleotide sequence or a non-self complementary semi-random sequence.

241. The method of any one of claims 151-240, wherein the target nucleic acids are mRNA, gDNA, RNA, tRNA, or a combination thereof.

242. The method of any one of claims 151-240, wherein the target nucleic acids are RNA, mRNA, or a combination thereof.

243. The method of any one of claims 151-242, wherein the extending of the capture nucleotide sequence in step (c) is carried out using a reverse transcriptase.

244. The method of any one of claims 151-243, wherein the target nucleic acids are polyadenylated prior to hybridization of the target nucleic acids to the capture nucleotide sequences.

245. The method of claim 94, wherein the target nucleic acids are polyadenylated using a poly(A) polymerase.

246. The method of claim 94, wherein the target nucleic acids are polyadenylated using chemical ligation or enzymatic ligation.

247. The method of any one of claim 153, 171, 199, or 222, wherein the amplifying comprises addition of a second clustering primer sequence to the one or more second complementary strands.

248. The method of claim 247, wherein the amplifying further comprises addition of an indexing sequence.

249. The method of claim 247 or claim 248, wherein the amplifying comprises index PCR during which a first primer hybridizes to the first clustering primer sequence and a second primer hybridizes to the adapter nucleotide sequence, wherein the second primer comprises the second clustering primer sequence.

250. The method of claim 249, wherein the second primer further comprises the indexing sequence.

251. The method of any one of claims 49-149, wherein the first adapter primer comprises a molecular identifier (SMI) sequence.

252. The method of claim 251, wherein the molecular identifier of the first adapter primer is incorporated during second strand cDNA synthesis.

253. The method of claim 251 or 252, wherein the SMI is a UMI.


SEQUENCE LISTING (18 sequences)

SEQ ID NO: 1 - DNA, 20 bp, synthetic construct
  source 1..20: mol_type = other DNA
aatgatacggcgaccaccga

SEQ ID NO: 2 - DNA, 29 bp, synthetic construct
  source 1..29: mol_type = other DNA
aatgatacggcgaccaccgagatctacac

SEQ ID NO: 3 - DNA, 20 bp, synthetic construct
  source 1..20: mol_type = other DNA
caagcagaagacggcatacg

SEQ ID NO: 4 - DNA, 24 bp, synthetic construct
  source 1..24: mol_type = other DNA
caagcagaagacggcatacgagat

SEQ ID NO: 5 - DNA, 20 bp, synthetic construct
  source 1..20: mol_type = other DNA
tcggtggtcgccgtatcatt

SEQ ID NO: 6 - DNA, 29 bp, synthetic construct
  source 1..29: mol_type = other DNA
gtgtagatctcggtggtcgccgtatcatt

SEQ ID NO: 7 - DNA, 20 bp, synthetic construct
  source 1..20: mol_type = other DNA
cgtatgccgtcttctgcttg

SEQ ID NO: 8 - DNA, 24 bp, synthetic construct
  source 1..24: mol_type = other DNA
atctcgtatgccgtcttctgcttg

SEQ ID NO: 9 - DNA, 15 bp, synthetic construct
  source 1..15: mol_type = other DNA
gtctcgtgggctcgg

SEQ ID NO: 10 - DNA, 15 bp, synthetic construct
  source 1..15: mol_type = other DNA
ccgagcccacgagac

SEQ ID NO: 11 - DNA, 35 bp, synthetic construct
  source 1..35: mol_type = other DNA
  variation 29: note = 'n' is a, c, g, or t/u
ttttttaatgatacggcgaccaccgaganctacac

SEQ ID NO: 12 - DNA, 33 bp, synthetic construct
  source 1..33: mol_type = other DNA
  variation 7..9: note = 'n' is a, c, g, or t/u
ttttttnnncaagcagaagacggcatacgagat

SEQ ID NO: 13 - DNA, 33 bp, synthetic construct
  source 1..33: mol_type = other DNA
acactctttccctacacgacgctcttccgatct

SEQ ID NO: 14 - DNA, 20 bp, synthetic construct
  source 1..20: mol_type = other DNA
acactctttccctacacgac

SEQ ID NO: 15 - DNA, 34 bp, synthetic construct
  source 1..34: mol_type = other DNA
gtgactggagttcagacgtgtgctcttccgatct

SEQ ID NO: 16 - DNA, 21 bp, synthetic construct
  source 1..21: mol_type = other DNA
gtgactggagttcagacgtgt

SEQ ID NO: 17 - DNA, 30 bp, synthetic construct
  source 1..30: mol_type = other DNA
  variation 1: note = Phosphorothioate
ttttttttttaatgatacggcgaccaccga

SEQ ID NO: 18 - DNA, 31 bp, synthetic construct
  source 1..31: mol_type = other DNA
  variation 1: note = Phosphorothioate
ttttttttttcaagcagaagacggcatacga